(19) United States
(12) Patent Application Publication
(10) Pub. No.: US 2026/0200854 A1
(43) Pub. Date: Jul. 16, 2026

(54) METHODS FOR THE SYNTHESIS OF COMPLEMENT FACTOR D INHIBITORS AND INTERMEDIATES THEREOF

(71) Applicant: ALEXION PHARMACEUTICALS, INC., Boston, MA (US)

(72) Inventors: Akihiro HASHIMOTO, Guilford, CT (US); Suresh Kumar TIPPARAJU, Arlington, MA (US)

(21) Appl. No.: 19/138,289

(22) PCT Filed: Dec. 14, 2023
(86) PCT No.: PCT/US2023/083992

§ 371 Date: Jun. 12, 2025

Related U.S. Application Data
(60) Provisional application No. 63/433,928, filed on Dec. 20, 2022.

(51) Int. Cl. C07D 213/73; C07D 401/14; C07D 409/14; C07D 471/04

(52) U.S. Cl. CPC C07D 213/73; C07D 401/14; C07D 409/14; C07D 471/04

(57) ABSTRACT

The present disclosure provides methods for the synthesis of complement factor D inhibitors and intermediates thereof.

### CROSS-REFERENCE TO RELATED APPLICATION

This application claims the benefit of priority to U.S. Application No. 63/433,928, filed Dec. 20, 2022, which is incorporated by reference herein for all purposes.

### BACKGROUND

The complement system is a part of the innate immune system which does not adapt to changes over the course of the host's life, but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. Complement Factor D plays an early and central role in activation of the alternative pathway of the complement cascade. Activation of the alternative complement pathway is initiated by spontaneous hydrolysis of a thioester bond within the C3 protein to produce C3(H₂O), which associates with Factor B to form the C3(H₂O)B complex. Complement Factor D acts to cleave Factor B within the C3(H₂O)B complex to form Ba and Bb. The Bb fragment remains associated with C3(H₂O) to form the alternative pathway C3 convertase C3(H₂O)Bb. Additionally, C3b generated by any of the C3 convertases also associates with Factor B to form C3bB, which Factor D cleaves to generate the later stage alternative pathway C3 convertase C3bBb. This latter form of the alternative pathway C3 convertase may provide important downstream amplification within all three of the defined complement pathways, leading ultimately to the recruitment and assembly of additional factors in the complement cascade pathway, including the cleavage of C5 to C5a and C5b. C5b acts in the assembly of factors C6, C7, C8, and C9 into the membrane attack complex, which can destroy pathogenic cells by lysing the cell.

The dysfunction of or excessive activation of complement has been linked to certain autoimmune, inflammatory, and neurodegenerative diseases, as well as ischemia-reperfusion injury and cancer. For example, activation of the alternative pathway of the complement cascade contributes to the production of C3a and C5a, both potent anaphylatoxins, which also have roles in a number of inflammatory disorders. Therefore, in some instances, it is desirable to decrease the response of the complement pathway, including the alternative complement pathway. Some examples of disorders mediated by the complement pathway include age-related macular degeneration (AMD), paroxysmal nocturnal hemoglobinuria (PNH), multiple sclerosis, and rheumatoid arthritis.

Additional complement-mediated disorders include those classified under component 3 glomerulopathy (C3G). C3G is a recently defined entity comprised of dense deposit disease (DDD) and C3 glomerulonephritis (C3GN) which encompasses a population of chronic kidney diseases wherein elevated activity of the alternative complement pathway and terminal complement pathway results in glomerular deposits made solely of complement C3 and no immunoglobulin (lg).

Immune-complex membranoproliferative glomerulonephritis (IC-MPGN) is a renal disease which shares many clinical, pathologic, genetic and laboratory features with C3G, and therefore can be considered a sister disease of C3G. In the majority of patients with IC-MPGN, an underlying disease or disorder—most commonly infections, autoimmune diseases, or monoclonal gammopathies—are identified to which the renal disease is secondary. Patients with idiopathic IC-MPGN can have low C3 and normal C4 levels, similar to those observed in C3G, as well as many of the same genetic or acquired factors that are associated with abnormal alternative pathway activity. Although there are current hypotheses suggesting that the majority of IC-MPGN is attributable to over activity of the classical pathway, those patients with a low C3 and a normal C4 are likely to have significant overactivity of the alternative pathway. IC-MPGN patients with a low C3 and a normal C4 may benefit from alternative pathway inhibition.

Other disorders that have been linked to the complement cascade include atypical hemolytic uremic syndrome (aHUS), hemolytic uremic syndrome (HUS), abdominal aortic aneurysm, hemodialysis complications, hemolytic anemia, or hemodialysis, neuromyelitis optica (NMO), myasthenia gravis (MG), fatty liver, nonalcoholic steatohepatitis (NASH), liver inflammation, cirrhosis, liver failure, dermatomyositis, and amyotrophic lateral sclerosis.

Factor D is an attractive target for inhibition or regulation of the complement cascade due to its early and essential role in the alternative complement pathway, and for its potential role in signal amplification within the classical and lectin complement pathways. Inhibition of Factor D effectively interrupts the pathway and attenuates the formation of the membrane attack complex.

To this end, a number of small molecule Factor D inhibitors have been developed and investigated for potential therapeutic uses. Examples of these Factor D inhibiting compounds methods of preparing them are described in PCT patent publications WO2017/035353A1, WO2020/041301A1, WO2021/168320A1, and WO2017/035409A1.

New methods for the synthesis of small molecule Factor D inhibitors and intermediates thereof are desirable.

### SUMMARY OF THE DISCLOSURE

The present disclosure generally relates to an improved method of preparing compounds useful for treating disorders mediated by complement factor D and intermediates thereof.

In particular, the present disclosure provides a method of preparing an aminopyridine of formula (A):

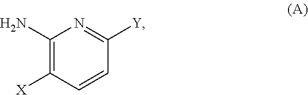

or an acid addition salt thereof, in which X and Y are as defined herein. The method includes (i) providing a compound of formula (B);

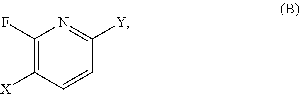

in which X and Y are as defined herein; (ii) coupling the compound of formula (B) to a compound of formula (C):

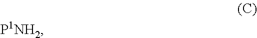

or a salt thereof, in which P¹ is as defined herein, to form a compound of formula (C):

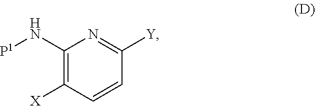

in which X, Y, and P¹ are as defined herein; and (iii) when P¹ is optionally substituted C₁-C₆ alkyl, reacting the compound of formula (C) with a Brønsted acid to form the compound of formula (A) or the acid addition salt thereof or, when P¹ is a N-protecting group, reacting the compound of formula (C) with a N-protecting-group-removing agent to form the compound of formula (A) or the acid addition salt thereof.

In some embodiments, P¹ is H.

In some embodiments, P¹ is optionally substituted C₁-C₆ alkyl, e.g., tert-butyl.

In some embodiments, P¹ is a N-protecting group, such as optionally substituted benzyl, e.g., 4-methoxybenzyl or 2,4-dimethoxybenzyl.

In some embodiments, in which P¹ is optionally substituted C₁-C₆ alkyl or a N-protecting group, the compound of formula (C) is reacted with a Brønsted acid, e.g., HCl, HBr, H₂SO₄, H₃PO₄, or trifluoroacetic acid. In some embodiments, the Brønsted acid is trifluoroacetic acid.

In some embodiments, the amine is N,N-diisopropylethylamine.

In some embodiments, the compound of formula (C) is reacted with a Brønsted acid (thus forming an acid addition salt of formula (A)), the method further includes reacting the acid addition salt of the compound of formula (A) with a base to obtain the compound of formula (A) as a free base. In some embodiments, the base is NaHCO₃ or K₂CO₃.

In some embodiments, said coupling the compound of formula (B) to the compound of formula (C) is performed with an excess of the compound of formula (B) or the salt thereof and the compound of formula (C) or the salt thereof (e.g., at a ratio of about 1:2).

In some embodiments, said providing the compound of formula (B) includes (i) providing a compound of formula (E):

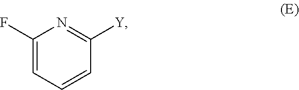

in which Y is as defined herein; and (ii) reacting the compound of formula (E) with a non-nucleophilic strong base, followed by an alkylation reagent including X to obtain the compound of formula (B).

In some embodiments, the non-nucleophilic strong base is a lithium amide, e.g., lithium diisopropylamide (LDA) or lithium bis(trimethylsilyl)amide (LiHMDS). In some embodiments, the non-nucleophilic strong base is a potassium amide, e.g., potassium bis(trimethylsilyl)amide (KHMDS).

In some embodiments, the alkylation agent is an alkyl halide, e.g., an alkyl iodide such as methyl iodide.

In some embodiments, the method further includes coupling the compound of formula (A) or the acid addition salt thereof to a compound of formula (I):

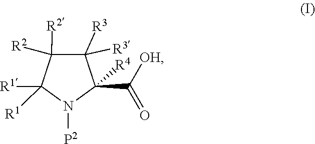

or a salt thereof, in which all variables are as defined herein, to form a compound of formula (II):

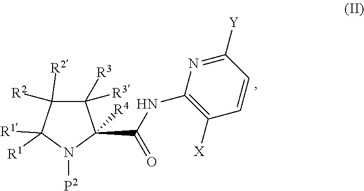

or a salt thereof, in which all variables are as defined herein.

In some embodiments, the method further includes reacting the compound of formula (II) or the salt thereof with a N-protecting-group-removing agent to form a compound of formula (III):

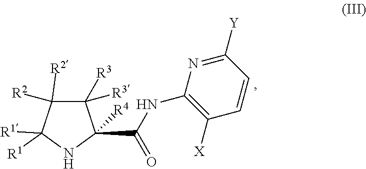

or a salt thereof, in which all variables are as defined herein.

In some embodiments, the method further includes coupling the compound of formula (III) or the salt thereof to a compound of formula (IV):

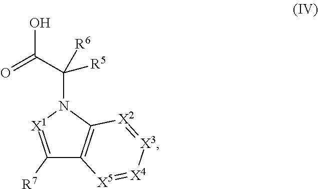

or a salt thereof, in which all variables are as defined herein, to form a compound of formula (V):

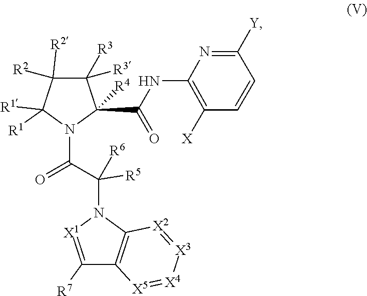

or a pharmaceutically acceptable salt thereof, in which all variables are as defined herein.

In some embodiments, P¹ is tert-butoxycarbonyl.

In some embodiments, the N-protecting-group-removing agent is hydrogen chloride, and said reacting the compound of formula (II) with the N-protecting-group-removing agent forms a hydrochloride salt of the compound of formula (III). In some embodiments, the hydrochloride salt of the compound of formula (III) is coupled to the compound of formula (IV) in dimethylformamide in the presence of 1-[bis(dimethylamino)methylene]-1H-1,2,3-triazolo[4,5-b]pyridinium 3-oxide hexafluorophosphate and N,N-diisopropylethylamine.

In some embodiments, the N-protecting-group-removing agent is hydrogen bromide, and said reacting the compound of formula (II) with the N-protecting-group-removing agent forms a hydrobromide salt of the compound of formula (III). In some embodiments, the hydrobromide salt of the compound of formula (III) is coupled to the compound of formula (IV) in acetonitrile in the presence of propanephosphonic acid anhydride and N,N-diisopropylethylamine.

In some embodiments, the N-protecting-group-removing agent is trifluoroacetic acid, and said reacting the compound of formula (II) with the N-protecting-group-removing agent forms a trifluoroacetic acid salt of the compound of formula (III). In some embodiments, the trifluoroacetic acid salt of the compound of formula (III) is coupled to the compound of formula (IV) in dimethylformamide in the presence of N, N-diisopropylethylamine and 1-[bis(dimethylamino)methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate.

In some embodiments, R² is fluoro. In some embodiments, R² is fluoro, and each of R¹, R^{1′}, R³, R^{3′}, and R⁴ is hydrogen.

In some embodiments,

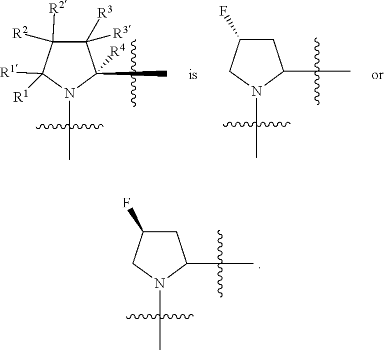

In some embodiments, R^{2′} is optionally substituted C₁-C₆ alkyl.

In some embodiments,

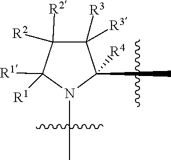

is

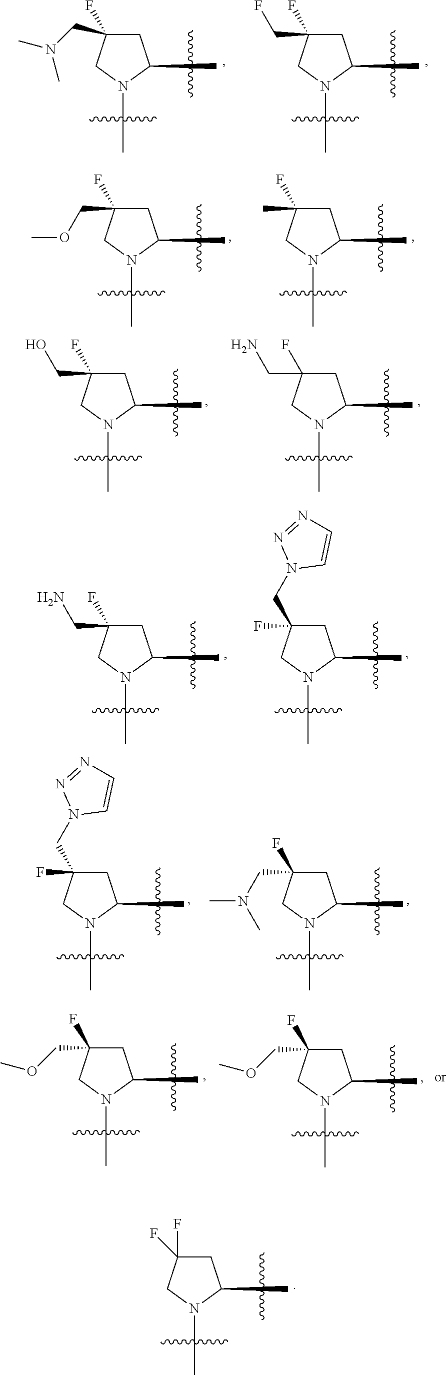

In some embodiments,

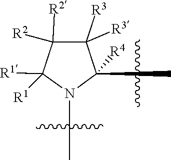

is

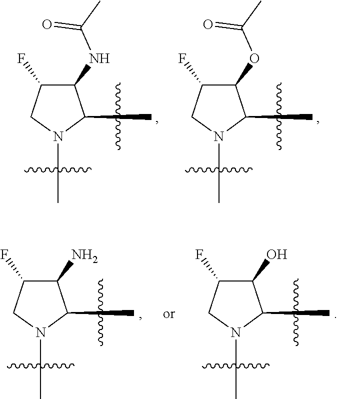

In some embodiments, R¹ and R², together with the atoms to which each is attached, form an optionally substituted C₃-C₆ cycloalkyl, e.g., optionally substituted cyclopropyl; and each of R^{1′}, R³, R^{3′}, and R⁴ is H.

In some embodiments, in which R¹ and R², together with the atoms to which each is attached, form an optionally substituted C₃-C₆ cycloalkyl, e.g., optionally substituted cyclopropyl; and each of R^{1′}, R³, R^{3′}, and R⁴ is H, R² is H. For example,

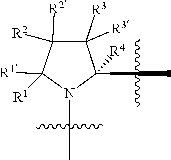

is

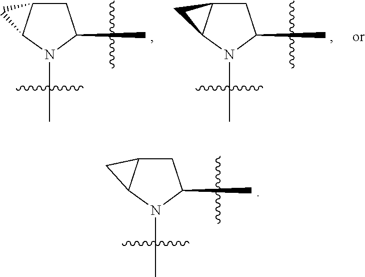

In some embodiments, in which R¹ and R², together with the atoms to which each is attached, form an optionally substituted C₃-C₆ cycloalkyl, e.g., optionally substituted cyclopropyl; and each of R^{1′}, R³, R^{3′}, and R⁴ is H, R^{2′} is optionally substituted C₁-C₆ alkyl. For example,

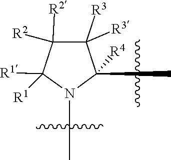

is

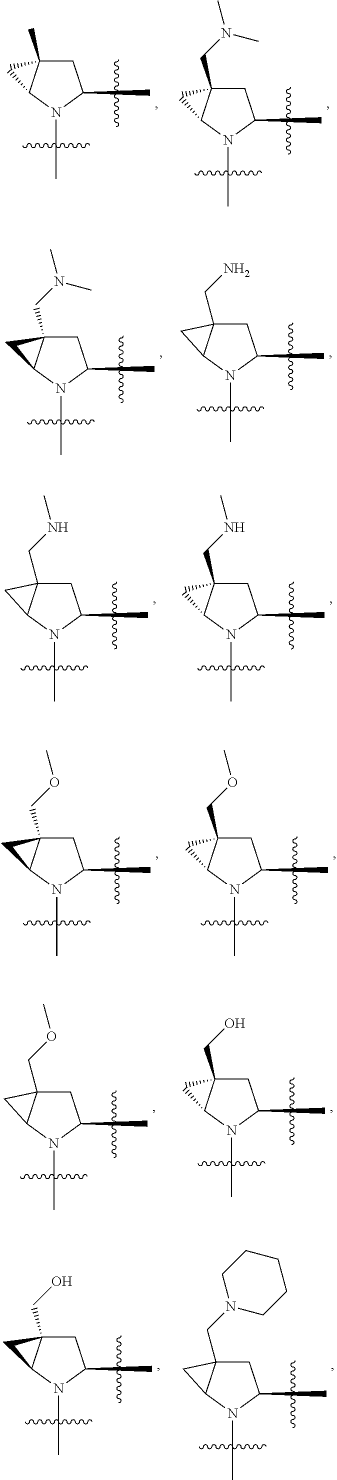

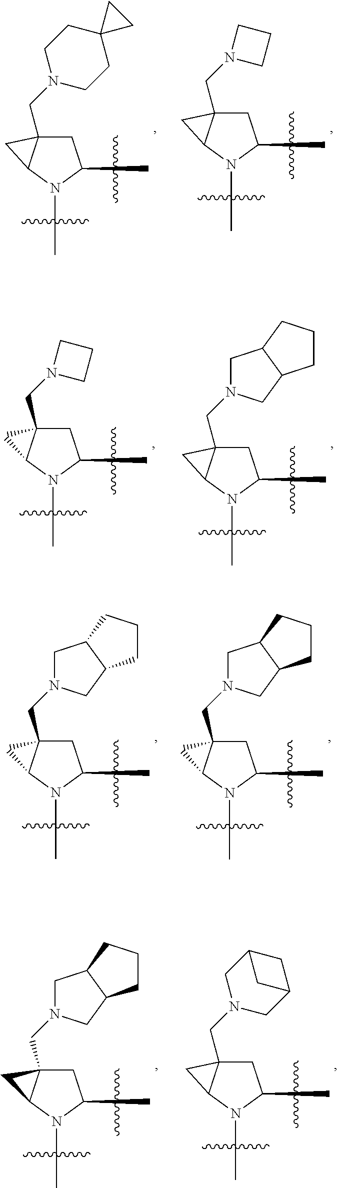

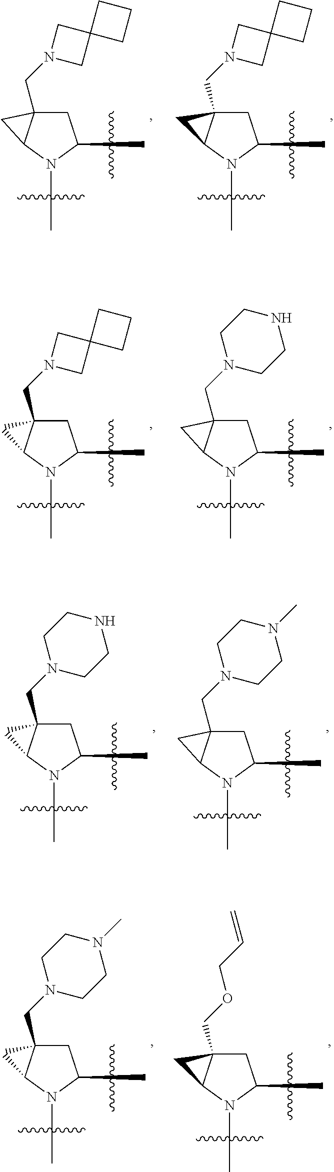

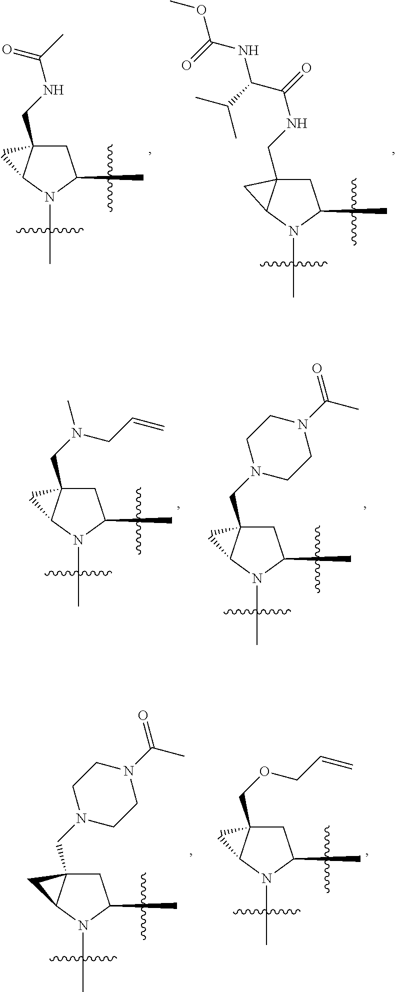

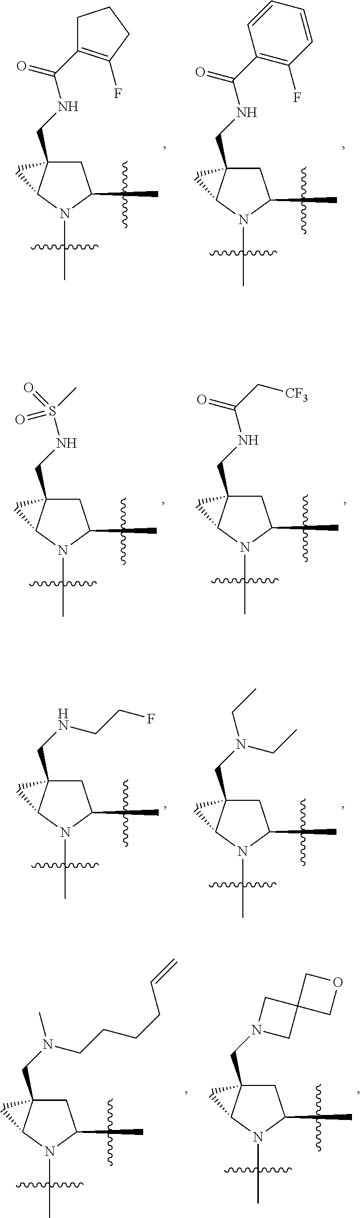

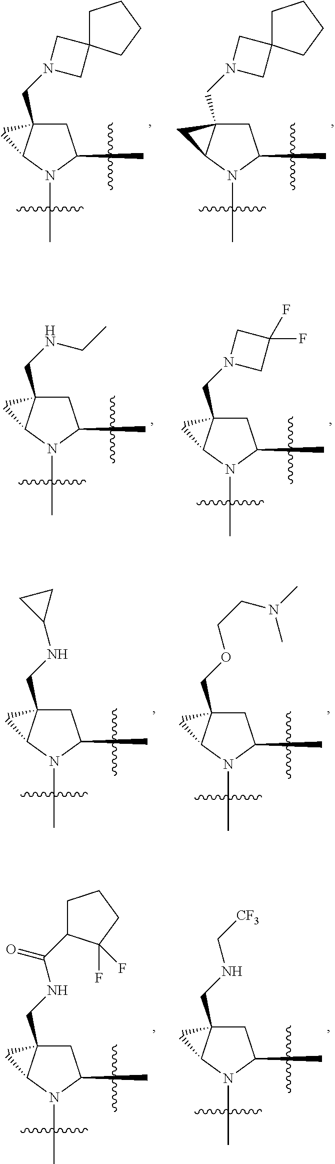

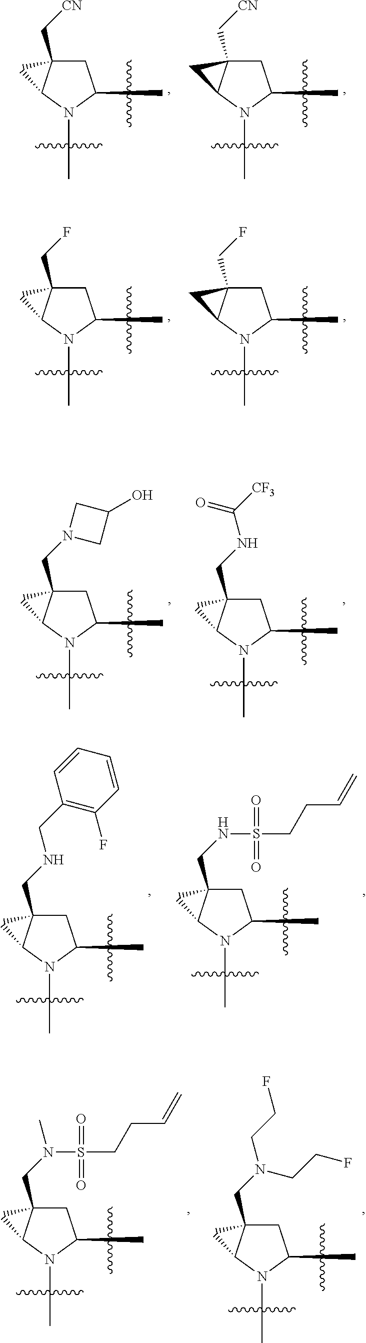

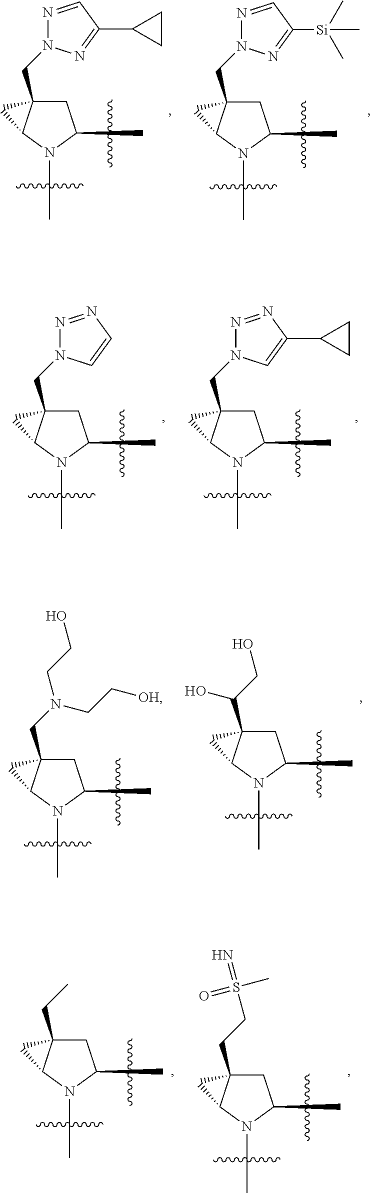

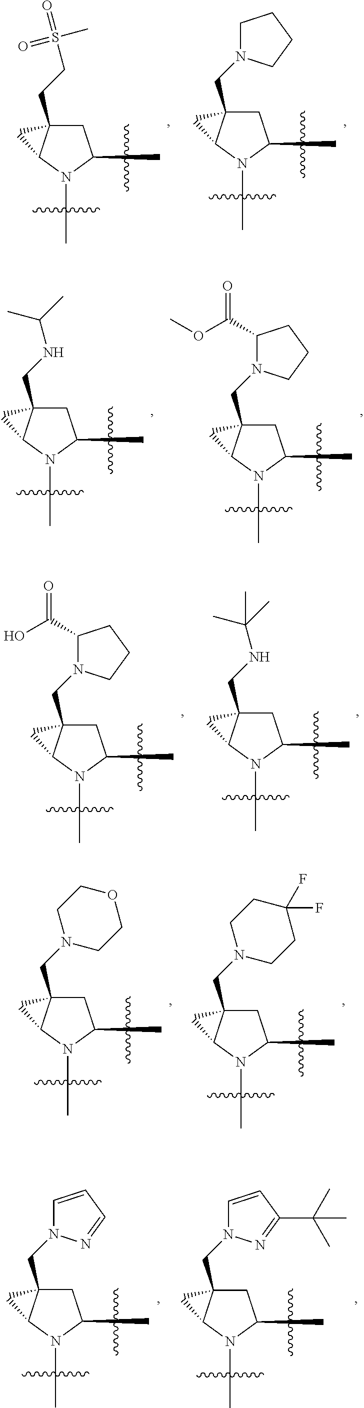

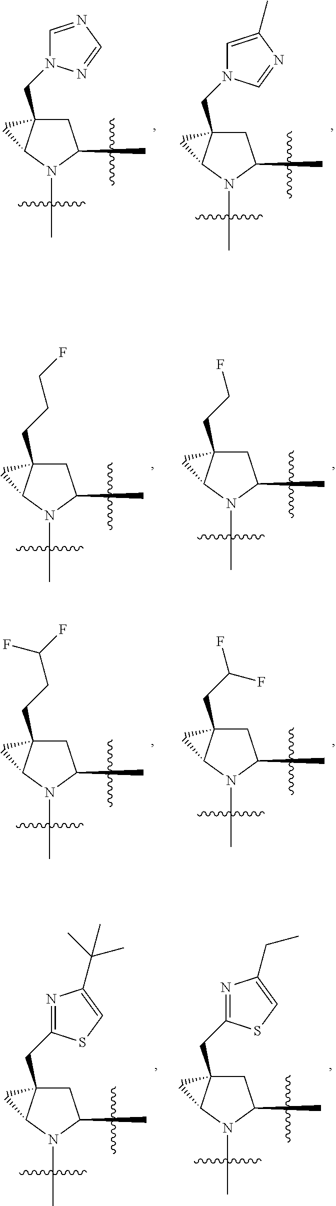

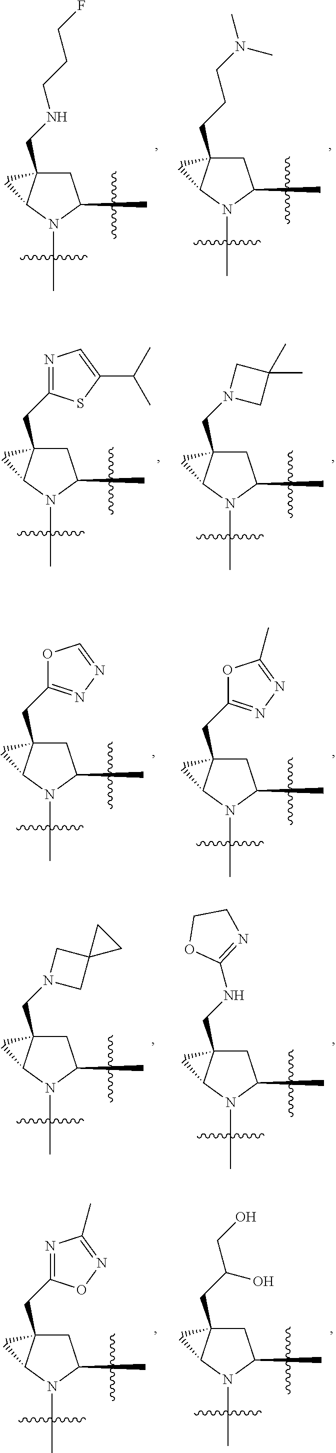

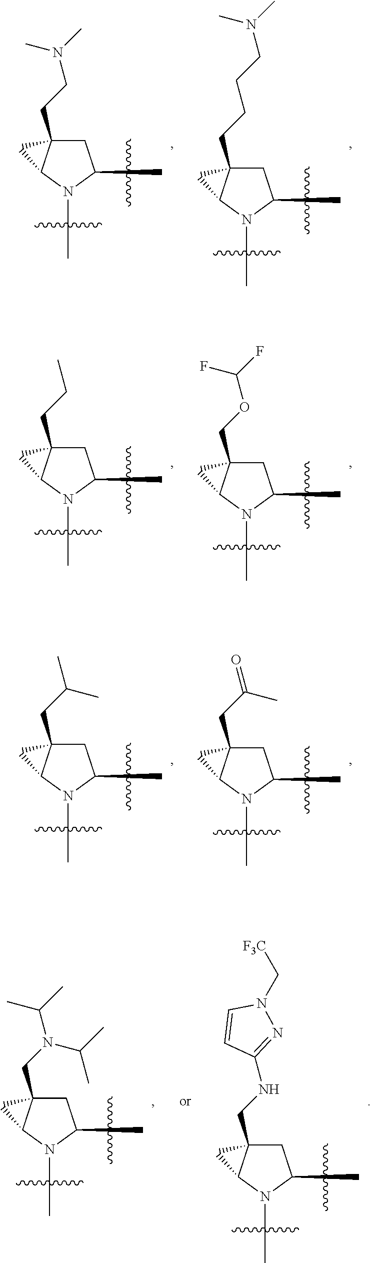

In some embodiments,

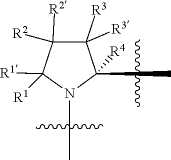

is

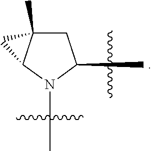 [SMILES: *N1[C@H](C)C[C@@]2(C)C[C@@H]12]

In some embodiments,

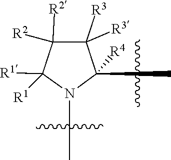

is

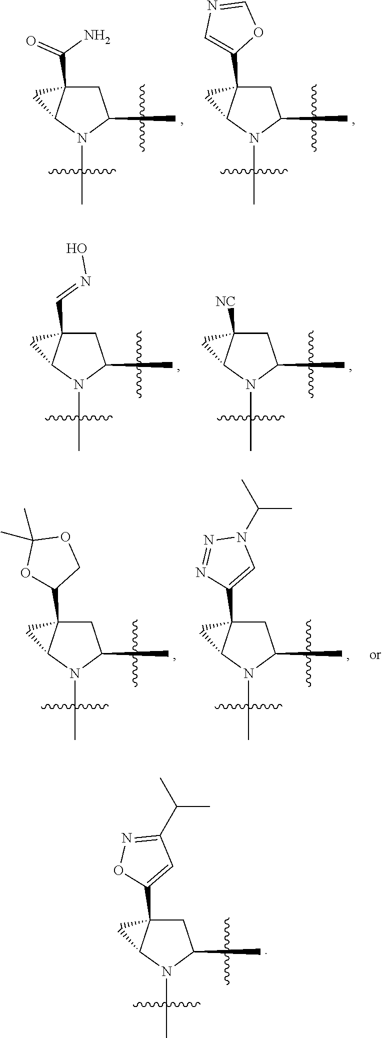

In some embodiments, R² and R³, together with the atoms to which each is attached, form an optionally substituted C₃-C₆ cycloalkyl, e.g., optionally substituted cyclopropyl, and each of R¹, R^{1′}, R^{2′}, R^{3′}, and R⁴ is H. For example,

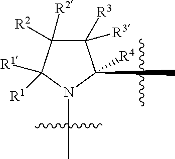

is

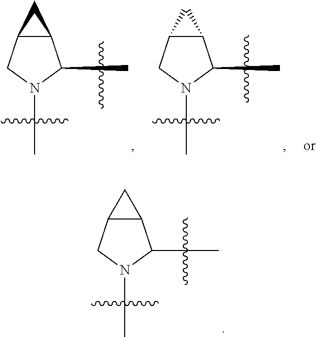

In some embodiments, R² and R⁴ combine to form optionally substituted C₁-C₂ alkylene. For example,

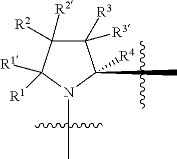

is

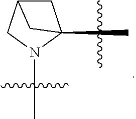

In some embodiments,

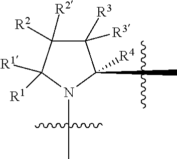

is

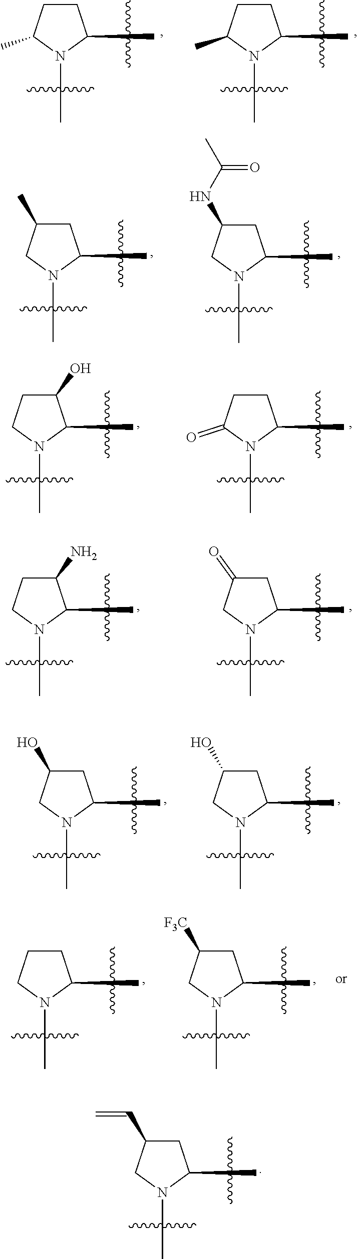

In some embodiments, X¹ is N. In some embodiments, X¹ is CRₑ, e.g., C(CH₃) or CH.

In some embodiments, X² is CR_{f}. In some embodiments, R^{f} is H or optionally substituted C₁-C₆ alkyl, e.g., X² is CH₃.

In some embodiments, X⁵ is CR_{f}, e.g., CH.

In some embodiments, X³ is CRₕ. In some embodiments, in which X³ is CRₕ, X⁴ is N. In some embodiments, in which X³ is CRₕ, X⁴ is CH.

In some embodiments, X⁴ is CRₕ. In some embodiments, in which X⁴ is CRₕ, X³ is N. In some embodiments, in which X⁴ is CRₕ, X³ is CH.

In some embodiments, R^{h} is optionally substituted 5- to 10-membered heteroaryl containing 1, 2, or 3 heteroatoms selected from N, O, and S.

In some embodiments, R^{h} is 6-membered heteroaryl containing 1, 2, or 3 heteroatoms selected from N, O, and S.

In some embodiments, R^{h} is optionally substituted pyrimidinyl, e.g.,

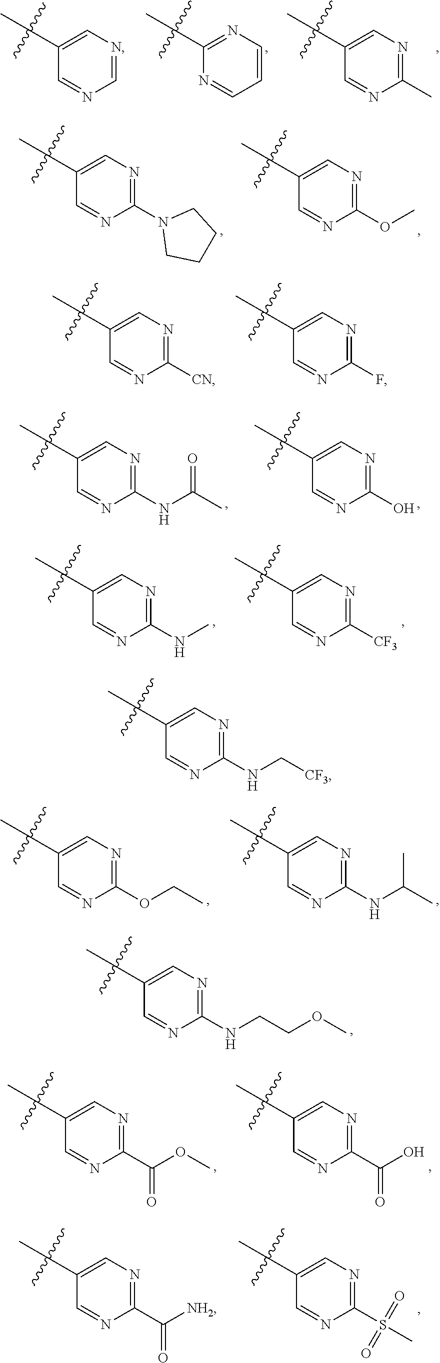

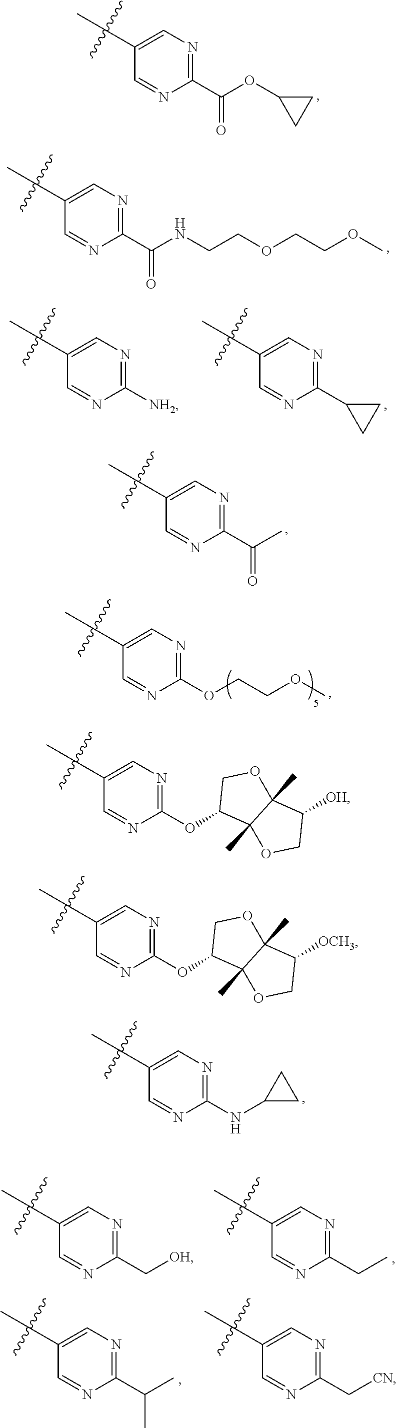

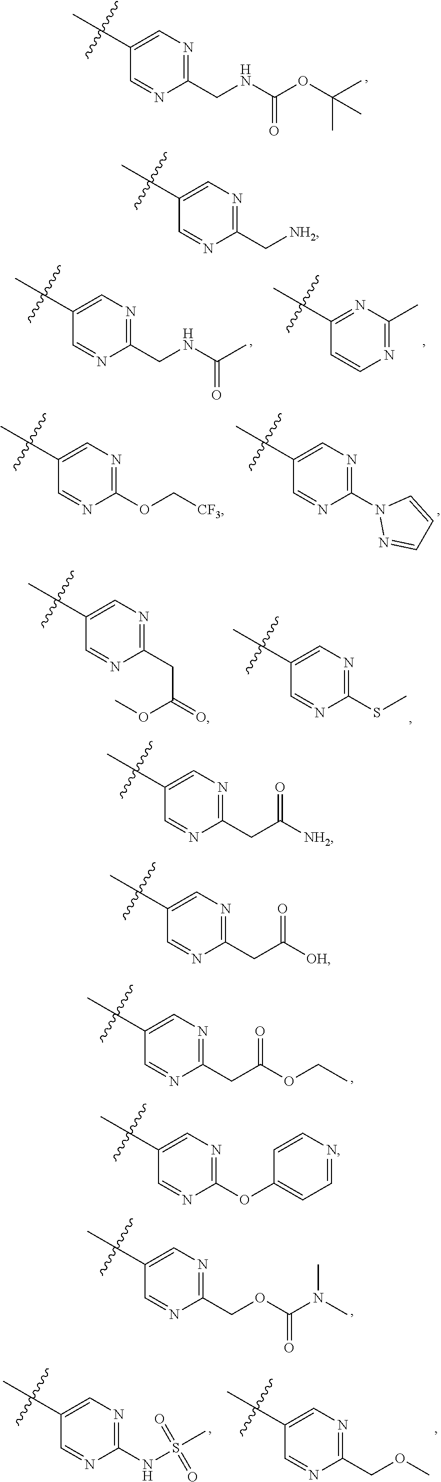

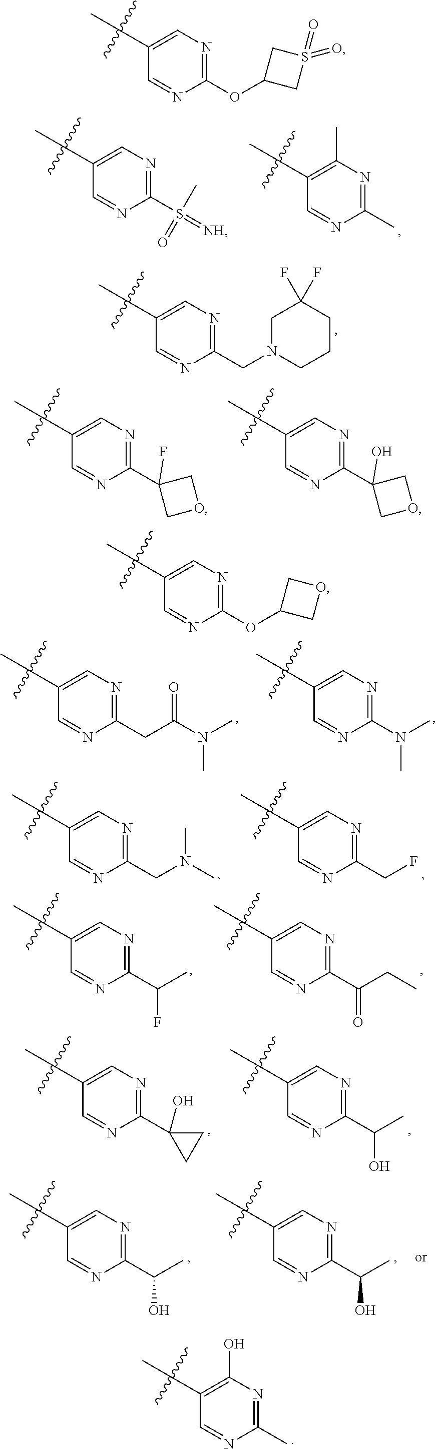

In some embodiments, R^{h} is

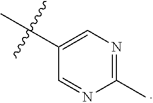 [SMILES: *c1cnc(C)nc1]

In some embodiments, R^{h} is

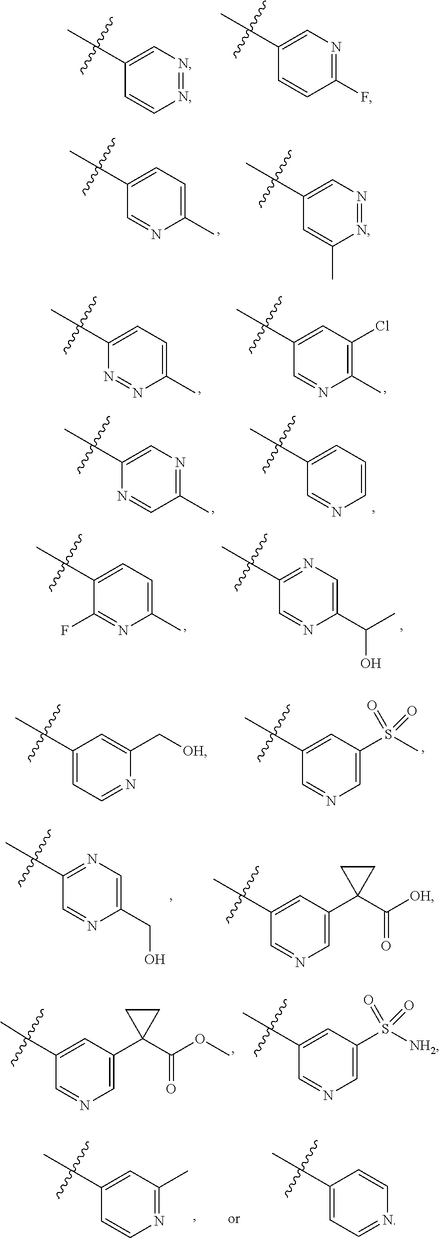

In some embodiments, R^{h} is optionally substituted 8- to 10-membered bicyclic heteroaryl containing 1, 2, or 3 heteroatoms selected from N, O, and S.

In some embodiments, R^{h} is optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl. For example, R^{h} is

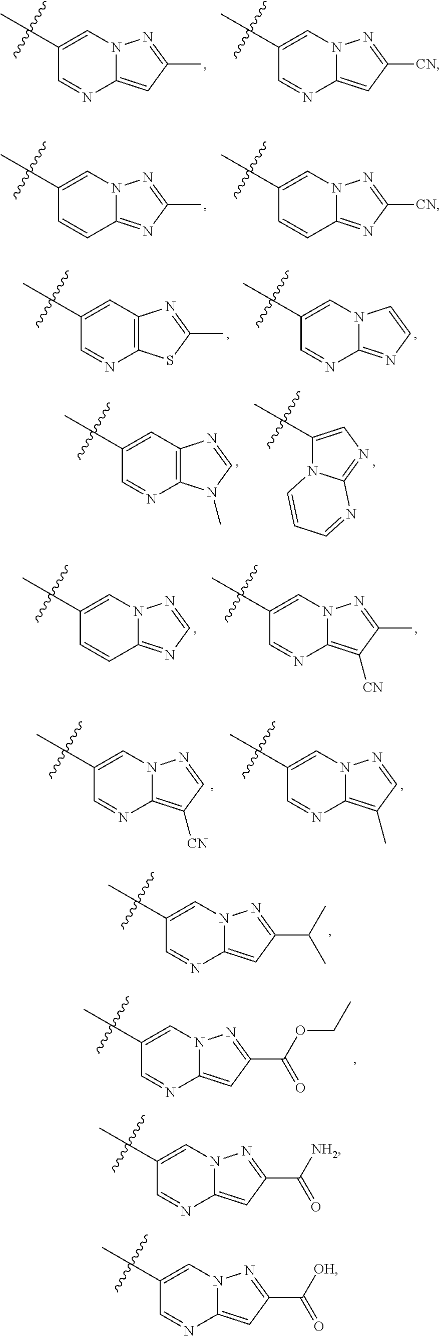

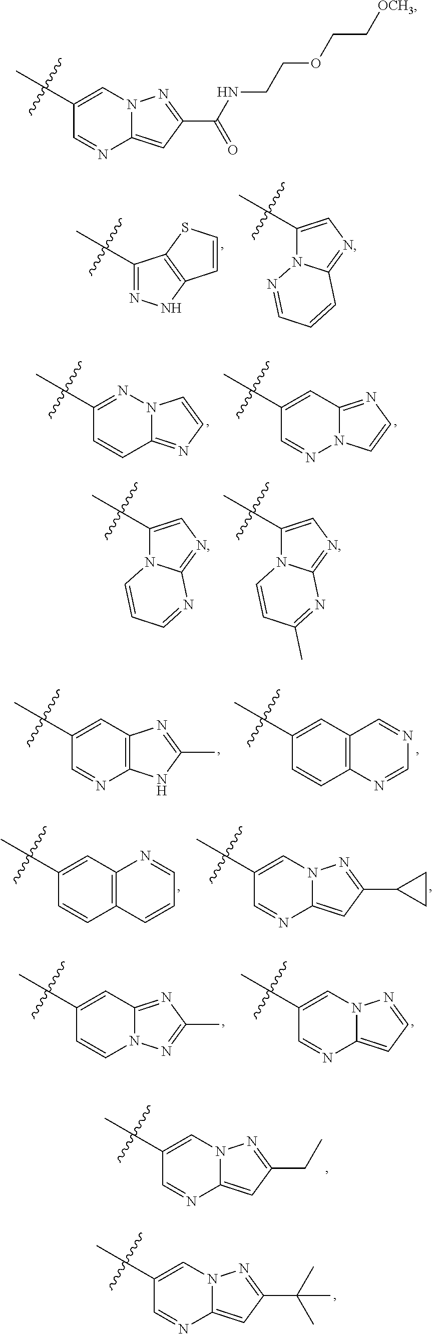

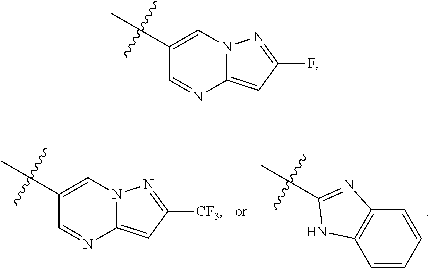 [SMILES: *c1cnc2cc(F)nn2c1 ; *c1cnc2cc(C(F)(F)F)nn2c1 ; *c1nc2ccccc2[nH]1]

In some embodiments, R^{h} is

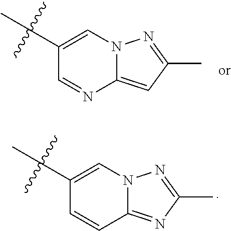 [SMILES: *c1cnc2cc(C)nn2c1 ; *c1ccc2nc(C)nn2c1]

In some embodiments, R^{h} is optionally substituted C₆-C₁₄ aryl, e.g., optionally substituted phenyl. For example, R^{h} is

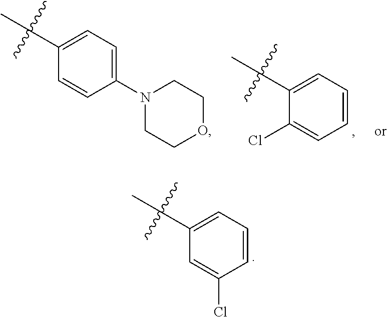 [SMILES: *c1ccc(N2CCOCC2)cc1 ; *c1ccccc1Cl ; *c1cccc(Cl)c1]

In some embodiments, R^{h} is optionally substituted 6- to 9-membered unsaturated heterocyclyl containing 1-4 heteroatoms selected from N, O, or S.

In some embodiments, in which Rₕ is optionally substituted 6- to 9-membered unsaturated heterocyclyl containing 1-4 heteroatoms selected from N, O, or S, R^{h} is bonded to the carbon atom to which it is attached through a carbon ring atom contained therein. For example, R^{h} is

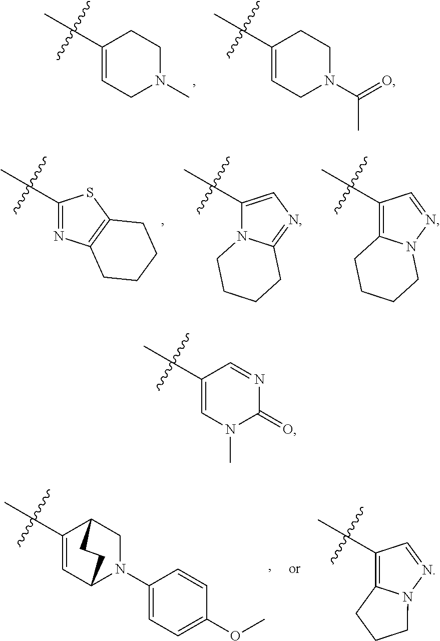

In some embodiments, R^{h} is

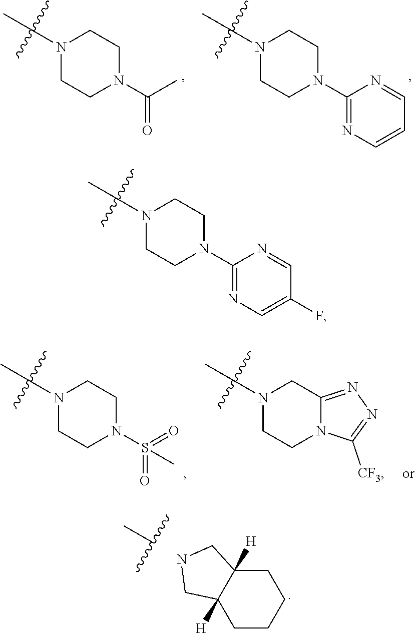

In some embodiments, R^{h} is optionally substituted 5-membered heteroaryl containing 1, 2, or 3 heteroatoms selected from N, O, and S, e.g.,

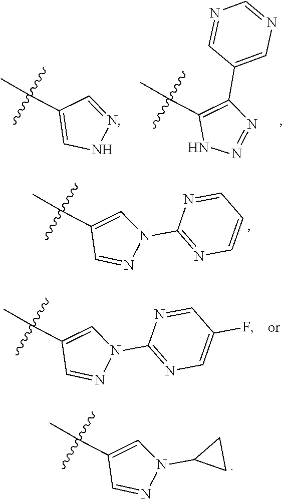

In some embodiments, R⁷ is —C(O)R_{d}, e.g.,

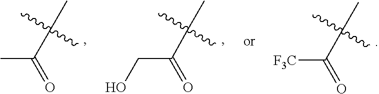

In some embodiments, R⁷ is

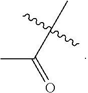 [SMILES: *C(C)=O]

In some embodiments, R⁷ is —C(O)NR_{c}R_{c}′, e.g.,

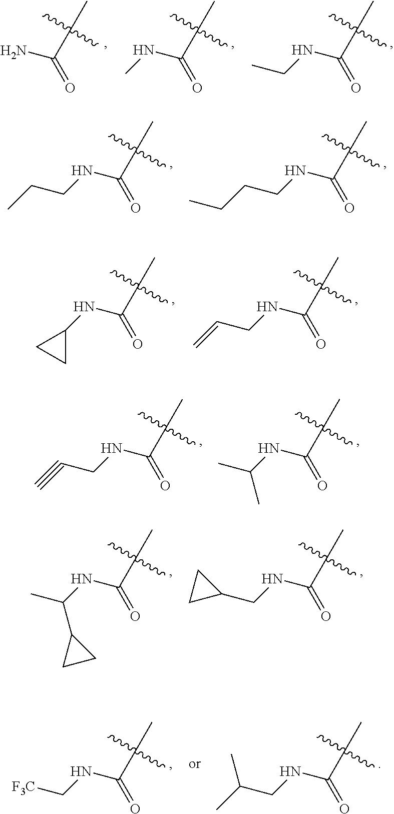

In some embodiments, R⁷ is

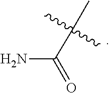 [SMILES: *C(N)=O]

In some embodiments, R⁷ is —C(O)OR_{d}, e.g., —C(O)OCH₃ or —C(O)OH. In some embodiments, R⁷ is optionally substituted C₁-C₆ alkyl, e.g.,

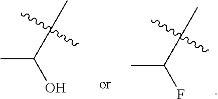

In some embodiments, R⁷ is

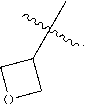 [SMILES: *C1COC1]

In some embodiments, R⁷ is cyano. In some embodiments, R⁷ is halo, e.g., F.

In some embodiments, R⁵ is H.

In some embodiments, R⁶ is H.

In some embodiments, Y is halo, e.g., Br.

In some embodiments, X is optionally substituted C₁-C₆ alkyl, e.g., methyl.

In some embodiments, the compound of formula (V) is a compound or disclosed in WO2017/035353A1 (the contents of which are incorporated herein by reference in their entirety) or a pharmaceutically acceptable salt thereof.

In some embodiments, the compound of formula (V) is a compound disclosed in WO2018/160889A1 (the contents of which are incorporated herein by reference in their entirety) or a pharmaceutically acceptable salt thereof.

In some embodiments, the compound of formula (V) is a compound disclosed in WO2020/041301A1 (the contents of which are incorporated herein by reference in their entirety) or a pharmaceutically acceptable salt thereof.

In some embodiments, the compound of formula (V) is a compound disclosed in WO2021/168320A1 (the contents of which are incorporated herein by reference in their entirety) or a pharmaceutically acceptable salt thereof.

In some embodiments, the compound of formula (V) is a compound disclosed in WO2017/035409A1 (the contents of which are incorporated herein by reference in their entirety) a pharmaceutically acceptable salt thereof.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative pharmaceutically acceptable salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. In the context of synthetic intermediates, e.g., the compounds of formula (A), acid addition salts may also include pharmaceutically undesirable salts, such as trifluoroacetate and the like.

The term “acyl,” as used herein, refers to a monovalent radical having the structure —COR, where R is alkyl, alkenyl, aryl, arylalkyl, heteroaryl, or heteroarylalkyl. Acyl can be optionally substituted as defined for each R group.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-12, 1-10, 1-8, 1-6, 1-4, or 1-2 carbon atoms (e.g., C₁-C₁₂, C₁-C₁₀, C₁-C₈, C₁-C₆, C₁-C₄, or C₁-C₂). Examples include, but are not limited to, methyl, ethyl, isobutyl, sec-butyl, and tert-butyl. In the context of alkylation reagents, “alkyl” may also include cycloalkyl, as defined herein.

The term “alkylation reagent,” as used herein, refers to reagent capable of donating an alkyl or cycloalkyl group (i.e., X in formula (A)) during the course of a reaction. Alkylation reagents are generally known in the art. Examples include, but are not limited to, alkyl halides (i.e., alkyl iodides such as methyl iodide, alkyl bromides such as methyl bromide, or alkyl chlorides such as chloromethane), dialkyl sulfates (e.g., dimethyl sulfate), trialkyloxonium tetrafluoroborates (e.g., trimethyloxonium tetrafluoroborate), alkyl p-toluenesulfonate (e.g., methyl p-tolueneulfonate), trialkyl orthoformate (e.g., trimethyl orthoformate), alkyl methanesulfonate (e.g., methyl methanesulfonate), dialkyl carbonate (e.g., dimethyl carbonate), 3-(trifluoromethyl)phenyltrimethylammonium hydroxide (e.g., 3-(trifluoromethyl)phenyltrimethylammonium hydroxide), and tetraalkylammonium salts (e.g., tetramethyl ammonium chloride and tetramethylammonium hydroxide).

The term “alkylene,” as used herein, refers to a divalent radical obtained by removing a hydrogen atom from a carbon atom of an alkyl group. The divalency of an alkylene group does not include the optional substituents on the alkylene group.

The term “alkenyl,” as used herein, refers to a branched or straight-chain monovalent unsaturated aliphatic radical containing at least one carbon-carbon double bond and no carbon-carbon triple bonds, and only C and H when unsubstituted. Monovalency of an alkenyl group does not include the optional substituents on the alkenyl group. For example, if an alkenyl group is attached to a compound, monovalency of the alkenyl group refers to its attachment to the compound and does not include any additional substituents that may be present on the alkenyl group. In some embodiments, the alkenyl group may contain, e.g., 2-12, 2-10, 2-8, 2-6, or 2-4 carbon atoms (e.g., C₂-C₁₂, C₂-C₁₀, C₂-C₈, C₂-C₆, or C₂-C₄). Examples include, but are not limited to, ethenyl, 1-propenyl, 2-propenyl, 1-methylethenyl, 1-butenyl, 2-butenyl, 3-butenyl, and the like.

The term “alkenyloxy,” as used herein, refers to a monovalent radical having the structure —O-alkenyl, in which “alkenyl” is as defined herein. Examples include, but are not limited to ethenyloxy, propenyloxy, and the like.

The term “alkoxy,” as used herein, refers to a monovalent radical having the structure —O-alkyl, in which “alkyl” is as defined herein. Examples include, but are not limited to methoxy, ethoxy, and n-butoxy, i-butoxy, t-butoxy, and the like.

The term “alkoxyalkyl,” as used herein, refers to a monovalent radical having the structure —R′OR″, in which R′ is alkylene, and R″ is alkyl. Alkoxyalkyl can be optionally substituted in the same manner as defined for each R′ and R″ group.

The term “alkynyl,” as used herein, refers to a branched or straight-chain monovalent unsaturated aliphatic radical containing at least one carbon-carbon triple bond and only C and H when unsubstituted. Monovalency of an alkynyl group does not include the optional substituents on the alkynyl group. For example, if an alkynyl group is attached to a compound, monovalency of the alkynyl group refers to its attachment to the compound and does not include any additional substituents that may be present on the alkynyl group. In some embodiments, the alkynyl group may contain, e.g., 2-12, 2-10, 2-8, 2-6, or 2-4 carbon atoms (e.g., C₂-C₁₂, C₂-C₁₀, C₂-C₈, C₂-C₆, or C₂-C₄). Examples include, but are not limited to, ethynyl, 1-propynyl, and 3-butynyl.

The term “alkylthioalkyl,” as used herein, refers to a monovalent radical having the structure —R′SR″, in which R′ is alkylene, and R″ is alkyl. Alkylthioalkyl can be optionally substituted in the same manner as defined for each R′ and R″ group.

The term “aryl,” as used herein, refers to a monovalent, monocyclic or fused ring bicyclic or polycyclic system which has the characteristics of aromaticity in terms of electron distribution throughout the ring system, e.g., phenyl, naphthyl, or phenanthryl. An aryl group may have, e.g., six to sixteen carbons (e.g., C₆-C₁₆ aryl, C₆-C₁₄ aryl, C₆-C₁₃ aryl, or C₆-C₁₀ aryl).

The term “arylalkoxy,” as used herein, refers to a monovalent radical having the structure —OR′R″, where R′ is alkylene, and R″ is aryl, in which “alkylene” and “aryl” are as defined herein. Arylalkoxy can be optionally substituted in the same manner as defined for each R′ and R″ group.

The term “arylalkoxyalkyl,” as used herein, refers to a monovalent radical having the structure —R′OR′R″, where each R′ is alkylene, and R″ is aryl. Arylalkoxyalkyl can be optionally substituted in the same manner as defined for each R′ and R″ group.

The term “arylalkyl,” as used herein, refers to a monovalent radical having the structure —R′R″, where R′ is alkylene, and R″ is aryl. Arylalkyl can be optionally substituted in the same manner as defined for each R′ and R″ group.

The term “aryloxy,” as used herein, refers to a monovalent radical having the structure —O-aryl, in which “aryl” is as defined herein.

The term “carbamate,” as used herein, represents a monovalent radical having the structure formula —OC(O)NR₂, in each R is independently H, optionally substituted alkyl, optionally substituted alkenyl, optionally substituted aryl, or optionally substituted arylalkyl.

The term “carbocyclyl,” as used herein, represents a monovalent, saturated or unsaturated non-aromatic cyclic group containing only C and H when unsubstituted. A carbocyclyl (e.g., a cycloalkyl or a cycloalkenyl) may have, e.g., three to fourteen carbons (e.g., a C₃-C₇, C₃-C₈, C₃-C₉, C₃-C₁₀, C₃-C₁₁, C₃-C₁₂, C₃-C₁₄ carbocyclyl). The term “carbocyclyl” also includes bicyclic and polycyclic (e.g., tricyclic and tetracyclic) fused ring structures.

The term “carbocyclyloxy,” as used herein, refers to a monovalent radical having the structure —O-carbocyclyl, e.g., a —O-cycloalkyl or a —O-cycloalkenyl radical. The terms “carbocyclyl,” “cycloalkyl,” and “cycloalkenyl” included in —O-carbocyclyl, —O-cycloalkyl, and —O-cycloalkenyl are as defined herein.

The term “carbonate,” as used herein, refers to a monovalent radical having the structure —OC(O)OR, where R is optionally substituted alkyl, optionally substituted alkenyl, optionally substituted aryl, or optionally substituted arylalkyl.

The term “cyano,” as used herein, refers to a monovalent radical having the structure —CN.

The term “cycloalkyl”, as used herein refers to a saturated carbocyclyl. Examples of cycloalkyls include, but are not limited to, cyclopropyl, cyclobutyl, cyclopentyl, cyclohexyl, and cycloheptyl. The term “cycloalkyl” also includes cyclic groups having a bridged multicyclic structure in which one or more carbons bridges two non-adjacent members of a monocyclic ring, e.g., bicyclo[2.2.1]heptyl and adamantyl. The term “cycloalkyl” also includes bicyclic, tricyclic, and tetracyclic fused ring structures, e.g., decalin and spirocyclic compounds.

The term “cycloalkyloxy,” as used herein, refers to a monovalent radical having the structure —O-cycloalkyl, in which “cycloalkyl” is as defined herein.

The term “cycloalkenyl,” as used herein, represents a monovalent, unsaturated carbocyclyl group that includes at least one carbon-carbon double bond, no carbon-carbon triple bond, only C and H when unsubstituted, and is not fully aromatic. A cycloalkenyl may have, e.g., four to fourteen carbons (e.g., a C₄-C₇, C₄-C₈, C₄-C₉, C₄-C₁₀, C₄-C₁₁, C₄-C₁₂, C₄-C₁₃, or C₄-C₁₄ cycloalkenyl). Exemplary cycloalkenyl groups include, but are not limited to, cyclopentenyl, cyclohexenyl, and cycloheptenyl. The term “cycloalkenyl” also includes cyclic groups having a bridged multicyclic structure in which one or more carbons bridges two non-adjacent members of a monocyclic ring, e.g., bicyclo[2.2.2]oct-2-ene. The term “cycloalkenyl” also includes fused ring bicyclic and multicyclic systems containing one or more double bonds, e.g., fluorene.

The term “ester,” as used herein, refers to a monovalent radical having the structure —OCOR, where R is alkyl, alkenyl, aryl, arylalkyl, heteroaryl, or heteroarylalkyl. Ester can be optionally substituted as defined for each R group.

The term “ether,” as used herein, refers to a monovalent radical having the structure —OR, where R is alkyl, alkenyl, arylalkyl, silyl, or 2-tetrahydropyranyl. Ether can be optionally substituted as defined for each R group.

The term “halo,” as used herein, refers to a fluorine (fluoro), chlorine (chloro), bromine (bromo), or iodine (iodo) radical.

The term “heteroarylalkyl,” as used herein, represents a monovalent radical of structure —R′R″, where R′ is alkylene, and R″ is heteroaryl. Heteroarylalkyl can be optionally substituted in the same manner as defined for each R′ and R″ group.

The term “heterocyclyl,” as used herein, represents a saturated or unsaturated monocyclic or fused ring bicyclic or polycyclic system having one or more carbon atoms and at least one heteroatom, e.g., one to four heteroatoms (e.g., one to four, one to three, one or two, one, two, three, or four heteroatoms), selected from N, O, and S. Heterocyclyl groups include both non-aromatic and aromatic systems. An aromatic heterocyclyl group is referred to as a “heteroaryl” group. In some embodiments, a heterocyclyl group is a 3- to 8-membered ring system, a 3- to 6-membered ring system, a 4- to 6-membered ring system, a 4- to 10-membered ring system, a 6- to 10-membered ring system, a 6- to 12-membered ring system, a 5-membered ring, or a 6-membered ring, or a ring or ring system having a number of ring atoms that fall within any of the above-mentioned ranges. Exemplary 5-membered heterocyclyl groups may have zero to two double bonds, and exemplary 6-membered heterocyclyl groups may have zero to three double bonds. Exemplary 5-membered groups include, for example, optionally substituted pyrrole, optionally substituted pyrazole, optionally substituted isoxazole, optionally substituted pyrrolidine, optionally substituted imidazole, optionally substituted thiazole, optionally substituted thiophene, optionally substituted thiolane, optionally substituted furan, optionally substituted tetrahydrofuran, optionally substituted diazole, optionally substituted triazole, optionally substituted tetrazole, optionally substituted oxazole, optionally substituted 1,3,4-oxadiazole, optionally substituted 1,3,4-thiadiazole, optionally substituted 1,2,3,4-oxatriazole, and optionally substituted 1,2,3,4-thiatriazole. Exemplary 6-membered heterocyclyl groups include, but are not limited to, optionally substituted pyridine, optionally substituted piperidine, optionally substituted piperazine, optionally substituted pyrimidine, optionally substituted pyrazine, optionally substituted pyridazine, optionally substituted triazine, optionally substituted 2H-pyran, optionally substituted 4H-pyran, and optionally substituted tetrahydropyran. Exemplary 7-membered heterocyclyl groups include, but are not limited to, optionally substituted azepine, optionally substituted 1,4-diazepine, optionally substituted thiepine, and optionally substituted 1,4-thiazepine. Exemplary 8- to 10-membered bicyclic groups include, but are not limited to, optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl. The term “hetercyclyl” also spirocyclic compounds.

The term “heterocyclyloxy,” as used herein, refers to a monovalent radical having the structure —O-heterocyclyl, in which “heterocyclyl” is as defined herein.

The term “hydroxyl protecting group,” as used herein, refers to any group capable of protecting the oxygen atom to which it is attached from reacting or bonding. A hydroxyl protecting group is installed by reacting a molecule including an unprotected hydroxyl group with a hydroxyl-protecting reagent. Hydroxyl protecting groups are known in the art, e.g., as described in Wuts, Greene's Protective Groups in Organic Synthesis, Wiley-Interscience, 4th Edition, 2006. Exemplary protecting groups (with the oxygen atom to which they are attached) are independently selected from the group consisting of esters, carbonates, carbamates, sulfonates, and ethers. In exemplary ester hydroxyl protecting groups, R of the acyl group is C₁-C₁₂ alkyl (e.g., C₁-C₈, C₁-C₆, C₁-C₄, C₂-C₇, C₃-C₁₂, and C₃-C₆ alkyl), C₂-C₁₂ alkenyl (e.g., C₂-C₈, C₂-C₆, C₂-C₄, C₃-C₁₂, and C₃-C₆ alkenyl), carbocyclic C₆-C₂₀ aryl (e.g., C₆-C₁₅, C₆-C₁₀, C₈-C₂₀, and C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉) heteroaryl(C₁-C₆)alkyl, or (C₁-C₆) heteroaryl(C₁-C₆)alkyl. Specific examples of acyl groups for use in esters include formyl, benzoylformyl, acetyl (e.g., unsubstituted or chloroacetyl, trifluoroacetyl, methoxyacetyl, triphenylmethoxyacetyl, and p-chlorophenoxyacetyl), 3-phenylpropionyl, 4-oxopentanoyl, 4,4-(ethylenedithio) pentanoyl, pivaloyl (Piv), vinylpivaloyl, crotonoyl, 4-methoxy-crotonoyl, naphthoyl (e.g., 1- or 2-naphthoyl), and benzoyl (e.g., unsubstituted or substituted, e.g., p-methoxybenzoyl, phthaloyl (including salts, such a triethylamine and potassium), p-bromobenzoyl, and 2,4,6-trimethylbenzoyl). As defined herein, any heteroaryl group present in an ester group has from 1 to 4 heteroatoms selected independently from O, N, and S. In exemplary carbonate hydroxyl protecting groups, R is C₁-C₁₂ alkyl (e.g., C₁-C₈, C₁-C₆, C₁-C₄, C₂-C₇, C₃-C₁₂, and C₃-C₆ alkyl), C₂-C₁₂ alkenyl (e.g., C₂-C₈, C₂-C₆, C₂-C₄, C₃-C₁₂, and C₃-C₆ alkenyl), carbocyclic C₆-C₂₀ aryl (e.g., C₆-C₁₅, C₆-C₁₀, C₈-C₂₀, and C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉) heteroaryl(C₁-C₆)alkyl, or (C₁-C₆) heteroaryl(C₁-C₆)alkyl. Specific examples include methyl, 9-fluorenylmethyl, ethyl, 2,2,2-trichloroethyl, 2-(trimethylsilyl)ethyl, 2-(phenylsulfonyl)ethyl, vinyl, allyl, t-butyl, p-nitrobenzyl, and benzyl carbonates. As defined herein, any heteroaryl group present in a carbonate group has from 1 to 4 heteroatoms selected independently from O, N, and S. In exemplary carbamate hydroxyl protecting groups, each R is independently H, C₁-C₁₂ alkyl (e.g., C₁-C₈, C₁-C₆, C₁-C₄, C₂-C₇, C₃-C₁₂, and C₃-C₆ alkyl), C₂-C₁₂ alkenyl (e.g., C₂-C₈, C₂-C₆, C₂-C₄, C₃-C₁₂, and C₃-C₆ alkenyl), carbocyclic C₆-C₂₀ aryl (e.g., C₆-C₁₅, C₆-C₁₀, C₈-C₂₀, and C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉) heteroaryl(C₁-C₆)alkyl, or (C₁-C₆) heteroaryl(C₁-C₆)alkyl. Specific examples include N-phenyl and N-methyl-N-(o-nitrophenyl) carbamates. As defined herein, any heteroaryl group present in a carbamate group has from 1 to 4 heteroatoms selected independently from O, N, and S. Exemplary ether hydroxyl protecting groups include C₁-C₁₂ alkyl (e.g., C₁-C₈, C₁-C₆, C₁-C₄, C₂-C₇, C₃-C₁₂, and C₃-C₆ alkyl), C₂₋₁₂ alkenyl (e.g., C₂-C₈, C₂-C₆, C₂-C₄, C₃-C₁₂, and C₃-C₆ alkenyl), (C₆-C₁₅)aryl(C₁-C₆)alkyl, (C₄-C₁₉) heteroaryl(C₁-C₆)alkyl, (C₁-C₆) heteroaryl(C₁-C₆)alkyl, (C₁-C₆)alkoxy (C₁-C₆)alkyl, (C₁-C₆)alkylthio(C₁-C₆)alkyl, (C₆-C₁₀)aryl(C₁-C₆)alkoxy (C₁-C₆)alkyl, and silyl (e.g., tri(C₁-C₆ alkyl)silyl, tri(C₆-C₁₀ aryl or C₁-C₆ heteroaryl)silyl, di(C₆-C₁₀ aryl or C₁-C₆ heteroaryl)(C₁-C₆ alkyl)silyl, and (C₆-C₁₀ aryl or C₁-C₆ heteroaryl)di(C₁-C₆ alkyl)silyl). Specific examples of alkylethers include methyl and t-butyl, and an example of an alkenyl ether is allyl. Ether hydroxyl protecting groups can be used to protect a carboxyl group (e.g., with a C₁₋₁₂ alkyl (e.g., C₁₋₈, C₁₋₆, C₁₋₄, C₂₋₇, C₃₋₁₂, and C₃₋₆ alkyl), (C₆₋₁₅)aryl(C₁₋₆)alkyl, (C₁₋₆)alkoxy (C₁₋₆)alkyl, (C₁₋₆)alkylthio(C₁₋₆)alkyl, or (C₆₋₁₀)aryl(C₁₋₆)alkoxy (C₁₋₆)alkyl). Examples of alkoxyalkyls and alkylthioalkyls that can be used as ether hydroxyl protecting groups include methoxymethyl, methylthiomethyl, (2-methoxyethoxy)methyl, and β-(trimethylsilyl) ethoxymethyl. Examples of arylalkyl groups that can be used as ether hydroxyl protecting groups include benzyl, p-methoxybenzyl (MPM), 3,4-dimethoxybenzyl, triphenylmethyl (trityl), o-nitrobenzyl, p-nitrobenzyl, p-halobenzyl, 2,6-dichlorobenzyl, p-cyanobenzyl, naphthylmethyl, and 2- and 4-picolyl ethers. Specific examples of silylethers include trimethylsilyl (TMS), triethylsilyl (TES), t-butyldimethylsilyl (TBS), t-butyldiphenylsilyl (TBDPS), triisopropylsilyl (TIPS), and triphenylsilyl (TPS) ethers. An example of an arylalkyloxyalkylether is benzyloxymethyl ether. As defined herein, any heteroaryl group present in an ether group has from 1 to 4 heteroatoms selected independently from O, N, and S. Alkyl groups, such as methyl, ethyl, isopropyl, n-propyl, t-butyl, n-butyl, and sec-butyl, and alkenyl groups, such as vinyl and allyl, can also be substituted with oxo, arylsulfonyl, halogen, and trialkylsilyl groups. Hydroxyl protecting groups can be installed and removed using methods known in the art.

The term “N-protecting group,” as used herein, refers to a group protecting a nitrogen atom in a molecule from participating in one or more undesirable reactions during chemical synthesis (e.g., oxidation reactions, or certain nucleophilic and electrophilic substitutions). An N-protecting group is installed by reacting the molecule including a nitrogen atom with an N-protecting reagent. Commonly used N-protecting groups and the corresponding N-protecting reagents are disclosed in Wuts, Greene's Protective Groups in Organic Synthesis, Wiley-Interscience, 4th Edition, 2006. Exemplary N-protecting groups include acyl (e.g., formyl, acetyl, trifluoroacetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, and 4-bromobenzoyl); sulfonyl-containing groups (e.g., benzenesulfonyl, p-toluenesulfonyl, o-nitrobenzenesulfonyl, and p-nitrobenzenesulfonyl); carbamate forming groups (e.g., benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl), arylalkyl (e.g., triphenylmethyl or benzyl, such as 4-methoxybenzyl and 2,4-dimethoxybenzyl); silyl groups (e.g., trimethylsilyl); and imine-forming groups (e.g., diphenylmethylene). Preferred N-protecting groups are acetyl, benzoyl, phenylsulfonyl, p-toluenesulfonyl, p-nitrobenzenesulfonyl, o-nitrobenzenesulfonyl, t-butyloxycarbonyl (Boc), and benzyloxycarbonyl (Cbz). N-protecting groups can be installed and removed using methods (i.e., with N-protecting-group removing agents) known in the art. In some embodiments, an allyl group is used as a protecting group for an anime, e.g., in the compound of formula (D), which may be removed using a palladium catalyst (such as palladium(0) bis(dibenzylideneacetone)) or by oxidation (e.g., with 2,3-dichloro-5,6-dicyano-1,4-benzoquinone).

The term “oxo,” as used herein, refers to a divalent oxygen atom represented by the structure ═O.

The term “silyl,” as used herein refers to a group of —SiR₃, in which each R is independently alkyl, alkenyl, aryl, or arylalkyl. Examples of silyl groups include tri(C₁-C₆ alkyl)silyl, tri(C₆-C₁₀ aryl or C₁-C₆ heteroaryl)silyl, di(C₆₋₁₀ aryl or C₁-C₆ heteroaryl)(C₁-C₆ alkyl)silyl, and (C₆-C₁₀ aryl or C₁-C₆ heteroaryl)di(C₁-C₆ alkyl)silyl. It will be understood that, when a silyl group includes two or more alkyl, alkenyl, aryl, heteroaryl, or arylalkyl groups, these groups are independently selected. As defined herein, any heteroaryl group present in a silyl group has from 1 to 4 heteroatoms selected independently from O, N, and S. Silyl can be optionally substituted in the same manner as defined for each R group.

The term “sulfonyl,” as defined herein, refers to a group of —S(O)₂R, where R is optionally substituted alkyl, optionally substituted alkenyl, optionally substituted aryl, optionally substituted arylalkyl, or silyl. In exemplary sulfonyls, R is C₁-C₁₂ alkyl (e.g., C₁-C₈, C₁-C₆, C₁-C₄, C₂-C₇, C₃-C₁₂, or C₃-C₆ alkyl), C₂-C₁₂ alkenyl (e.g., C₂-C₈, C₂-C₆, C₂-C₄, C₃-C₁₂, or C₅-C₆ alkenyl), carbocyclic C₆-C₂₀ aryl (e.g., C₆-C₁₅, C₆-C₁₀, C₈-C₂₀, or C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉)heteroaryl(C₁-C₆)alkyl, or (C₁-C₆) heteroaryl(C₁-C₆)alkyl. As defined herein, any heteroaryl group present in a sulfonate group has from 1 to 4 heteroatoms selected independently from O, N, and S.

The term “thioalkyl,” as used herein, refers to a monovalent radical having the structure —S-alkyl, in which “alkyl” is as defined herein.

The phrase “optionally substituted X,” as used herein, is intended to be equivalent to “X wherein X is optionally substituted” (e.g., “alkyl, wherein said alkyl is optionally substituted”). It is not intended to mean that the feature “X” (e.g., alkyl) per se is optional. The term “optionally substituted,” as used herein, refers to having 0, 1, or more substituents (e.g., 0-10, 0-9, 0-8, 0-7, 0-6, 0-5, 0-4, 0-3, 0-2, 0 or 1, 0, 1, 2, 3, 4, 5, 6, 7, 8, 9, or 10 substituents).

Alkyl, alkylene, alkenyl, alkynyl, carbocyclyl, carbocyclyloxy, cycloalkyl, cycloalkyloxy, cycloalkenyl, cycloalkenyloxy, aryl, aryloxy, heteroaryl, heteroaryloxy, heterocyclyl, and heterocyclyloxy groups may be substituted with one or more of carbocyclyl, carbocyclyloxy, cycloalkyl, cycloalkyloxy, cycloalkenyl, cycloalkenyloxy, aryl, aryloxy, heteroaryl, heteroaryloxy, heterocyclyl, and heterocyclyloxy; halo; OH; oxo, cyano; alkoxy; alkenyloxy; thioalkyl; NO₂; N₃; NRR′; wherein each of R and R′ is, independently, H, alkyl, alkenyl, alkynyl, cycloalkyl, cycloalkenyl, aryl, or heterocyclyl; SO₂R, wherein R is H, alkyl or aryl; SO₂NRR′, wherein each of R and R′ is, independently, H, alkyl, or aryl; and NRSO₂R′, wherein each of R and R′ is, independently, H, alkyl, alkenyl, alkynyl, cycloalkyl, cycloalkenyl, aryl, or heterocyclyl. Carbocyclyl, carbocyclyloxy, cycloalkyl, cycloalkyloxy, cycloalkenyl, cycloalkenyloxy, aryl, aryloxy, heteroaryl, heteroaryloxy, heterocyclyl, and heterocyclyloxy groups may also be substituted with alkyl, alkenyl, alkynyl, or silyl. In some embodiments, a substituent is further substituted as described herein. For example, a C₆ aryl group, i.e., phenyl, may be substituted with an alkyl group, which may be further substituted with a heterocyclyl group. As another example, a C₁ alkyl group, i.e., methyl, may be substituted with oxo to form a formyl group, and may be further substituted with hydroxyl to form a carboxyl group.

### DETAILED DESCRIPTION OF THE DISCLOSURE

The present disclosure provides methods for the synthesis of small molecule complement factor D inhibitors and intermediates thereof. The complement factor D inhibitors are compounds of formula (V):

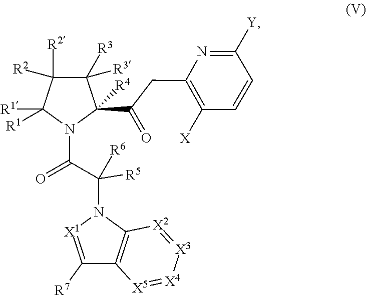

or pharmaceutically acceptable salts thereof, in which variables X, Y, R¹, R^{1′}, R², R^{2′}, R³, R^{3′}, R⁴, R⁵, R⁶, R⁷, and X¹—X⁵ are as defined herein. Exemplary compounds of formula (V) are described in, e.g., WO2017/035353A1, WO2020/041301A1, WO2021/168320A1, and WO2017/035409A1, the contents of which are incorporated herein by reference in their entirety.

### Compounds of Formula (A)

This present disclosure is based, in part, on the discovery of an alternative route to synthesizing compounds of formula (A):

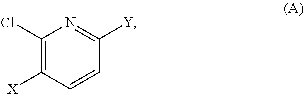

or an acid addition salt thereof, in which X is optionally substituted C₁-C₆ alkyl or optionally substituted C₃-C₈ cycloalkyl; and Y is halo; cyano; OH; optionally substituted C₁-C₆ alkyl; optionally substituted C₁-C₆ alkoxyl; optionally substituted C₃-C₈ cycloalkyl; optionally substituted C₆-C₁₀ aryl; optionally substituted 4- to 10-membered saturated or unsaturated non-aromatic heterocyclyl containing 1-4 heteroatoms selected from N, O, and S; optionally substituted 5- to 10-membered heteroaryl containing 1, 2, or 3 heteroatoms selected from N, O, and S; optionally substituted C₁-C₆ thioalkyl; OR_{y1}, wherein R_{y1} is optionally substituted 5- to 10-membered heteroaryl containing 1, 2, or 3 heteroatoms selected from N, O, and S or optionally substituted 4- to 10-membered saturated or unsaturated non-aromatic heterocyclyl containing 1-4 heteroatoms selected from N, O, and S; or C(O)R_{y2}, wherein R_{y2} is H, optionally substituted C₁-C₆ alkyl, optionally substituted C₃-C₈ cycloalkyl, or optionally substituted C₆-C₁₀ aryl.

The method includes (i) providing a compound of formula (B);

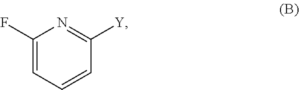

in which X and Y are as defined for formula (A), (ii) coupling the compound of formula (B) or the salt thereof to a compound of formula (C):

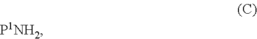

in which P¹ is H, optionally substituted C₁-C₆ alkyl, or a N-protecting group, e.g., in the presence of a base. to form a compound of formula (C):

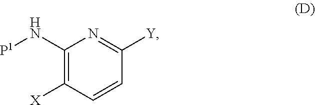

or a salt thereof, wherein all variables are as defined for formulas (B) and (C); and (iii) (a) when P¹ is optionally substituted C₁-C₆ alkyl (e.g., an unsubstituted C₁-C₆ alkyl such as tert-butyl), reacting the compound of formula (C) or the salt thereof with a Brønsted acid to form the compound of formula (A) or the salt thereof; or (b) when P¹ is a N-protecting group, reacting the compound of formula (C) with a N-protecting-group-removing agent to form the compound of formula (A) or the salt thereof. The base can be a non-nucleophilic amine, amidine, or an aromatic nitrogenous base (e.g., N,N-diisopropylethylamine (DIPEA or Hünig's base), 1,8-diazabicycloundec-7-ene (DBU), 1,5-diazabicyclo(4.3.0) non-5-ene (DBN), triethylamine pyridine, N,N-dimethylaniline, N-methylmorpholine, or N-methylimidazole) or an inorganic base (e.g., CsCO₃, K₂CO₃, NaHCO₃, NaOH, KOH, LiOH, or Na₂PO₄).

In embodiments where P¹ is a N-protecting group, it can typically be removed with a Brønsted acid (e.g., HCl, HBr, H₂SO₄, H₃PO₄, or trifluoracetic acid) provide the compound of formula (A) as an acid addition salt. In embodiments in which an acid addition salt of the compound of formula (A) is produced, it can then be reacted with a base to provide the compound of formula (A) as a free base. Suitable bases for neutralizing the acid addition salt of formula (A), e.g., K₂CO₃, NaHCO₃, NH₄OH, NaOH, LiOH, and others, are known in the art. In some embodiments, the base is NaHCO₃. In some embodiments, the base is K₂CO₃.

In some embodiments, X is C₁-C₆ alkyl. In some embodiments, X is methyl. In some embodiments, X is ethyl. In some embodiments, X is isopropyl. In some embodiments, X is C₃-C₈ cycloalkyl.

The present method offers greater flexibility in preparing aminopyridine fragments for their introduction into the structure of factor D inhibitors (e.g., those of formula (V)) as the use of the compounds of formula (B), in which the fluoro group in the 2-position activates the C—H bond in the 3-position of the pyridine, allow for the installation of substituents such as alkyl and cycloalkyl groups in the 3-position after deprotonation with a non-nucleophilic base. By contrast, current methods of preparing compounds of formula (A) require the use of pyridine compounds substitute in the 3-position as reactants. Further, the present method eliminates that use of expensive reagents such as trifluoromethanesulfonic anhydride, which reduces the manufacturing costs of the compounds of formula (A).

### Compounds of Formula (B)

In some embodiments, the compound of formula (B) is prepared from a compound (E):

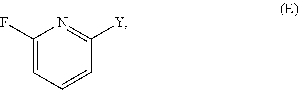

in which Y is as defined for formula (A). The compound of formula (B) may be prepared by reacting the compound of formula (E) with a non-nucleophilic strong base, followed by an alkylation reagent containing the group X to obtain the compound of formula (B).

The compound of formula (E) is deprotonated by the non-nucleophilic strong base in the 3-position (ortho to the fluoro group), which allows for the addition of group X as a substituted using a compound of formula (F), a compound of formula (G), or a combination as the source of the X group. Suitable non-nucleophilic strong bases include, but are not limited to, lithium/potassium amides (e.g., lithium diisopropylamide or a silicon-based amide such as potassium bis(trimethysilyl)amide or lithium bis(trimethysilyl)amide), sodium hydride, potassium hydride, and lithium tetramethylpiperidine. In some embodiments, the non-nucleophilic strong base is a lithium amide. In some embodiments, the non-nucleophilic strong base is lithium diisopropylamide. In some embodiments, the non-nucleophilic strong base is potassium bis(trimethylsilyl)amide. In some embodiments, the non-nucleophilic strong base is lithium bis(trimethylsilyl)amide.

The compound of formula (E), after reacting with a non-nucleophilic strong base, is reacted with an alkylation reagent to introduce the X group to the 3-position of the compound of formula (E). Suitable alkylation reagents, including but not limited to those disclosed herein, are generally known in the art. In some embodiments, the alkylation reagent is an alkyl halide, i.e., an alkyl iodide of formula X-I, an alkyl bromide of formula X—Br, or an alkyl chloride of formula X—Cl, in which X is as defined herein.

In some embodiments, the compound of formula (E), after reacting with a strong base, is reacted with a compound of formula (G). In some embodiments, the compound of formula (F) is a di(C₁-C₆ alkyl) sulfate. In some embodiments, the compound of formula (F) is diethyl sulfate. In some embodiments, the compound of formula (F) is dimethyl sulfate.

### Compounds of Formulas (I), (II), and (III)

In some embodiments, the compound of formula (A) (e.g., 6-bromo-3-methylpyridin-2-amine) or the acid addition salt thereof is coupled to a compound of formula (I):

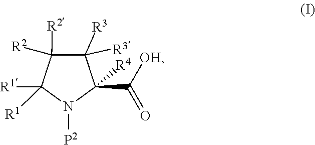

or a salt thereof, in which each of R¹, R^{1′}, R², R², R³, R^{3′}, and R⁴ is, independently, H; cyano; halo; OH; nitro; optionally substituted C₁-C₆ alkyl; optionally substituted C₂-C₆ alkenyl; C₁-C₆ alkoxy; C₁-C₆ thioalkyl; optionally substituted C₃-C₈ carbocyclyl; optionally substituted C₃-C₈ carbocyclyloxy; —NRₐRₐ′; —C(O)NRₐRₐ′; —OC(O)NRₐRₐ′; —NRₐC(O)R_{b}; —NRₐC(O)OR_{b}; —C(O)R_{b}; —C(O)OR_{b}; —C═NR_{b}; or C═NOH, in which each of Rₐ, Rₐ′, and R_{b} is independently selected from H, optionally substituted C₁-C₆ alkyl, optionally substituted C₂-C₆ alkenyl, optionally substituted C₂-C₆ alkynyl, and optionally substituted C₃-C₈ carbocyclyl; or R¹ and R², together with the atoms to which each is attached, form an optionally substituted C₃-C₆ cycloalkyl; or R² and R³, together with the atoms to which each is attached, form an optionally substituted C₃-C₆ cycloalkyl; or R¹ and R₃ or R² and R⁴ combine to form an optionally substituted C₁-C₂ alkylene; or R¹ and R^{1′}, R² and R^{2′}, or R³ and R^{3′} combine to form oxo; and P² is a N-protecting group, to form a compound of formula (II):

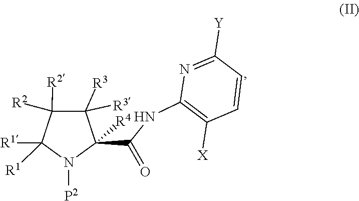

or a salt thereof, in which all variables are as defined for formula (I).

The compound of formula (II) or the salt thereof may be further deprotected using a N-protecting-group-removing agent to provide a compound of formula (III):

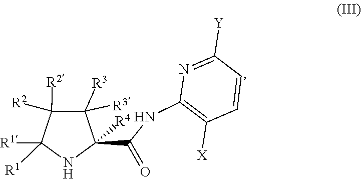

or a salt thereof, in which all variables are as defined for formula (II). In some embodiments, the N-protecting-group-removing agent is hydrogen chloride, and said reacting the compound of formula (II) with the N-protecting-group-removing agent forms a hydrochloride salt of the compound of formula (III). In some embodiments, the N-protecting-group-removing agent is hydrogen bromide, and said reacting the compound of formula (II) with the N-protecting-group-removing agent forms a hydrobromide salt of the compound of formula (III). In some embodiments, the N-protecting-group-removing agent is trifluoroacetic acid, and said reacting the compound of formula (II) with the N-protecting-group-removing agent forms a trifluoroacetic acid salt of the compound of formula (III).

### Compounds of Formulas (IV) and (V)

In some embodiments, the compound of formula (III) or the salt thereof is further coupled to a compound of formula (IV):

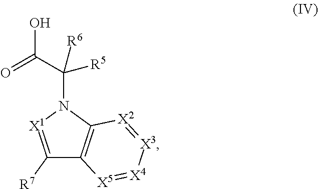

or a salt thereof, in which each of R⁵ and R⁶ is independently H or optionally substituted C₁-C₆ alkyl; R⁷ is H; halo; OH; NH₂; cyano; optionally substituted C₁-C₆ alkyl; optionally substituted C₂-C₆ alkenyl; optionally substituted 3- to 8-membered heterocyclyl; —C(O)NR_{c}R_{c}′, in which each of R_{c} and R_{c}′ is independently H, optionally substituted C₁-C₆ alkyl, optionally substituted C₂-C₆ alkenyl, optionally substituted C₂-C₆ alkynyl, optionally substituted C₁-C₆ alkoxyl, or optionally substituted C₃-C₈ cycloalkyl; —C(O)R_{d}; —OC(O)R_{d}; or
—C(O)OR_{d}, in which R_{d}, in each instance, is selected from H, optionally substituted C₁-C₆ alkyl, optionally substituted C₁-C₆ alkoxy, and optionally substituted C₃-C₈ carbocyclyl; X¹ is N or CRₑ, in which Rₑ is H, halo, optionally substituted C₁-C₆ alkyl, or optionally substituted C₁-C₆ alkoxy; each of X² and X⁵ is independently N or CR_{f}, wherein each R_{f} is independently selected from H, halo, cyano, optionally substituted C₁-C₆ alkyl, optionally substituted C₁-C₆ alkoxy, optionally substituted C₃-C₈ carbocyclyl, and optionally substituted 5- to 8-membered heteroaryl; and each of X³ and X⁴ is independently selected from N, CR_{g}, and CRₕ, in which R_{g} is selected from H, halo, cyano, optionally substituted C₁-C₆ alkyl, optionally substituted C₁-C₆ alkoxy, and —C(O)ORᵢ, in which Rᵢ is H or optionally substituted C₁-C₆ alkyl; and Rₕ is selected from optionally substituted C₄-C₁₀ aryl, optionally substituted 5- to 10-membered heteroaryl containing 1, 2, or 3 heteroatoms selected from N, O, and S, and optionally substituted 4- to 10-membered saturated or unsaturated non-aromatic heterocyclyl containing 1-4 heteroatoms selected from N, O, and S; wherein at least one of X³ and X⁴ is CRₕ, to form a compound of formula (V):

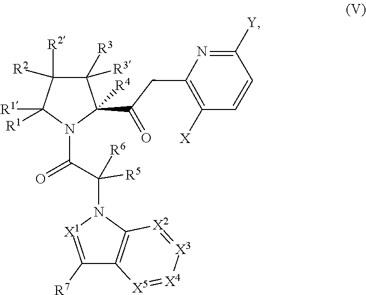

or a pharmaceutically acceptable salt thereof, in which X and Y are as defined for formula (A); R¹, R^{1′}, R², R^{2′}, R³, R^{3′}, and R⁴ are as defined for formula (II); and all other variables are as defined for formula (IV).

In some embodiments, the hydrochloride salt of the compound of formula (III) is coupled to the compound of formula (IV) in dimethylformamide in the presence of 1-[bis(dimethylamino)methylene]-1H-1,2,3-triazolo[4,5-b]pyridinium 3-oxide hexafluorophosphate and N, N-diisopropylethylamine.

In some embodiments, the hydrobromide salt of the compound of formula (III) is coupled to the compound of formula (IV) in acetonitrile in the presence of propanephosphonic acid anhydride and N, N-diisopropylethylamine. In some embodiments, the trifluoroacetic acid salt of the compound of formula (III) is coupled to the compound of formula (IV) in dimethylformamide in the presence of N, N-diisopropylethylamine and 1-[bis(dimethylamino)methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate.

Exemplary compounds of formulas (I), (II), (III), (IV), and (V) and processes for preparing the compounds of formulas (II), (III), (IV), and (V) are disclosed in, e.g., WO2017/035353A1, WO2020/041301A1, WO2021/168320A1, and WO2017/035409A1, the entire contents of which are incorporated herein by reference.

In some embodiments, the compound of formula (V) is a compound disclosed in WO2017/035353A1. For example, the compound of formula (V) is a compound of Table 1 or a pharmaceutically acceptable salt thereof.

TABLE 1

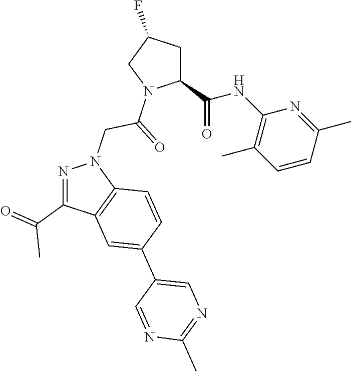 [SMILES: CC(=O)c1nn(CC(=O)N2C[C@H](F)C[C@H]2C(=O)Nc2nc(C)ccc2C)c2ccc(-c3cnc(C)nc3)cc12]

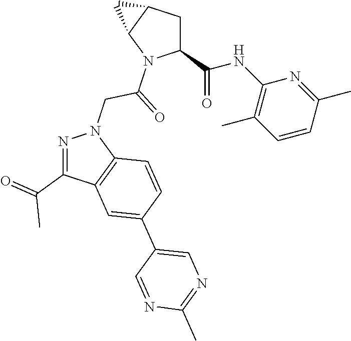 [SMILES: CC(=O)c1nn(CC(=O)N2[C@@H]3C[C@@H]3C[C@H]2C(=O)Nc2nc(C)ccc2C)c2ccc(-c3cnc(C)nc3)cc12]

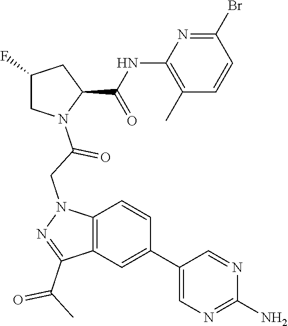 [SMILES: CC(=O)c1nn(CC(=O)N2C[C@H](F)C[C@H]2C(=O)Nc2nc(Br)ccc2C)c2ccc(-c3cnc(N)nc3)cc12]

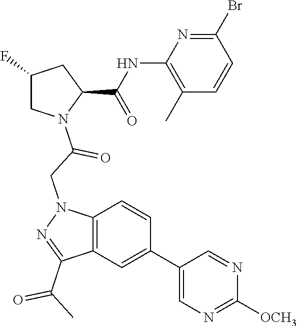 [SMILES: COc1ncc(-c2ccc3c(c2)c(C(C)=O)nn3CC(=O)N2C[C@H](F)C[C@H]2C(=O)Nc2nc(Br)ccc2C)cn1]

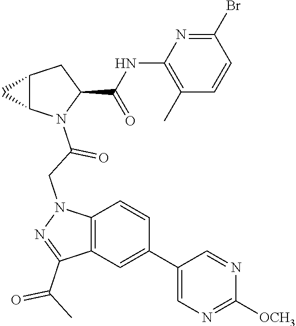 [SMILES: COc1ncc(-c2ccc3c(c2)c(C(C)=O)nn3CC(=O)N2[C@@H]3C[C@@H]3C[C@H]2C(=O)Nc2nc(Br)ccc2C)cn1]

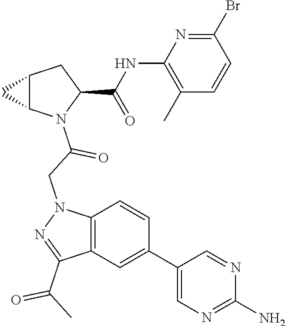 [SMILES: CC(=O)c1nn(CC(=O)N2[C@@H]3C[C@@H]3C[C@H]2C(=O)Nc2nc(Br)ccc2C)c2ccc(-c3cnc(N)nc3)cc12]

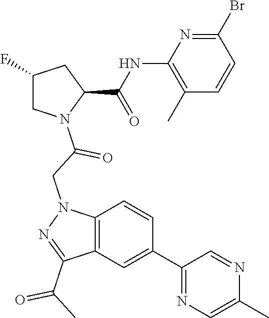 [SMILES: CC(=O)c1nn(CC(=O)N2C[C@H](F)C[C@H]2C(=O)Nc2nc(Br)ccc2C)c2ccc(-c3cnc(C)cn3)cc12]

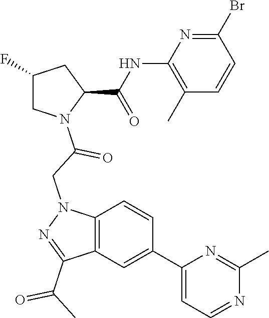 [SMILES: CC(=O)c1nn(CC(=O)N2C[C@H](F)C[C@H]2C(=O)Nc2nc(Br)ccc2C)c2ccc(-c3ccnc(C)n3)cc12]

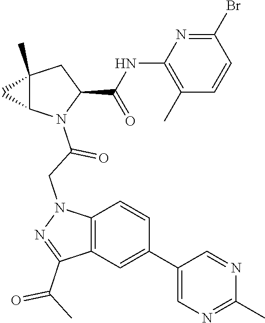 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

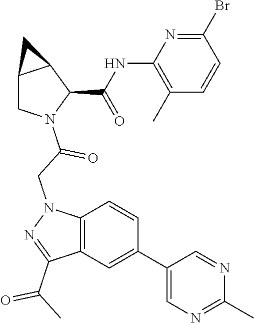 [SMILES: CC(=O)c1nn(CC(=O)N2C[C@H]3C[C@H]3[C@H]2C(=O)Nc2nc(Br)ccc2C)c2ccc(-c3cnc(C)nc3)cc12]

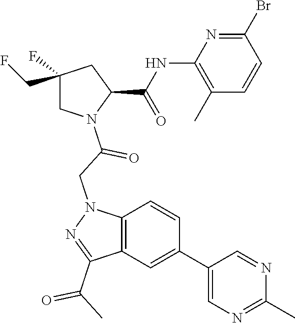 [SMILES: CC(=O)c1nn(CC(=O)N2C[C@@](F)(CF)C[C@H]2C(=O)Nc2nc(Br)ccc2C)c2ccc(-c3cnc(C)nc3)cc12]

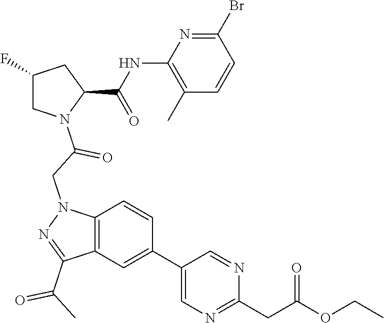 [SMILES: CCOC(=O)Cc1ncc(-c2ccc3c(c2)c(C(C)=O)nn3CC(=O)N2C[C@H](F)C[C@H]2C(=O)Nc2nc(Br)ccc2C)cn1]

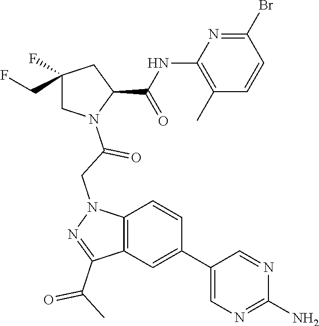 [SMILES: CC(=O)c1nn(CC(=O)N2C[C@@](F)(CF)C[C@H]2C(=O)Nc2nc(Br)ccc2C)c2ccc(-c3cnc(N)nc3)cc12]

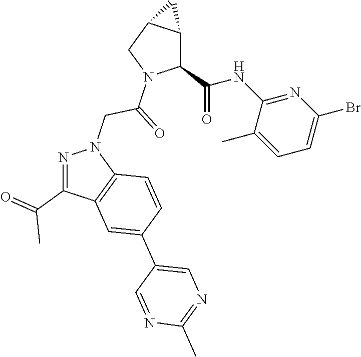 [SMILES: CC(=O)c1nn(CC(=O)N2C[C@@H]3C[C@@H]3[C@H]2C(=O)Nc2nc(Br)ccc2C)c2ccc(-c3cnc(C)nc3)cc12]

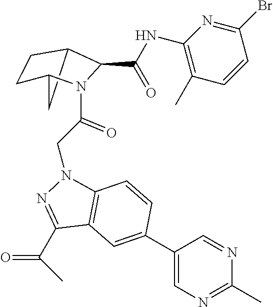 [SMILES: CC(=O)c1nn(CC(=O)N2C3CCC(C3)[C@H]2C(=O)Nc2nc(Br)ccc2C)c2ccc(-c3cnc(C)nc3)cc12]

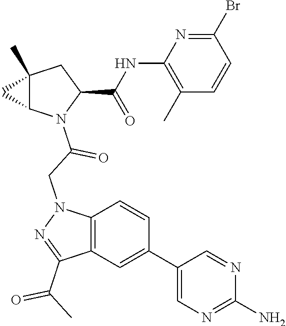 [SMILES: CC(=O)c1nn(CC(=O)N2[C@H](C(=O)Nc3nc(Br)ccc3C)C[C@@]3(C)C[C@@H]23)c2ccc(-c3cnc(N)nc3)cc12]

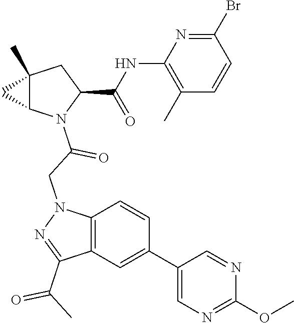 [SMILES: COc1ncc(-c2ccc3c(c2)c(C(C)=O)nn3CC(=O)N2[C@H](C(=O)Nc3nc(Br)ccc3C)C[C@@]3(C)C[C@@H]23)cn1]

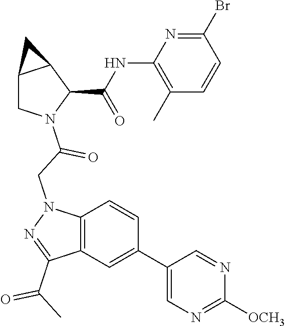 [SMILES: COc1ncc(-c2ccc3c(c2)c(C(C)=O)nn3CC(=O)N2C[C@H]3C[C@H]3[C@H]2C(=O)Nc2nc(Br)ccc2C)cn1]

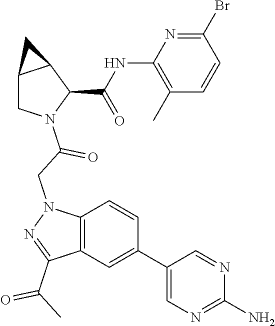 [SMILES: CC(=O)c1nn(CC(=O)N2C[C@H]3C[C@H]3[C@H]2C(=O)Nc2nc(Br)ccc2C)c2ccc(-c3cnc(N)nc3)cc12]

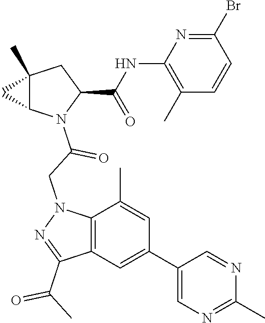 [SMILES: CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]

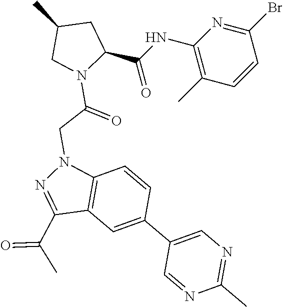 [SMILES: CC(=O)c1nn(CC(=O)N2C[C@@H](C)C[C@H]2C(=O)Nc2nc(Br)ccc2C)c2ccc(-c3cnc(C)nc3)cc12]

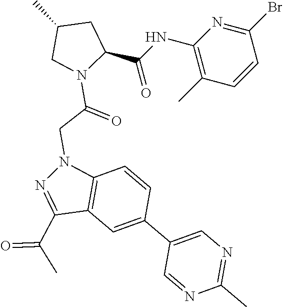 [SMILES: CC(=O)c1nn(CC(=O)N2C[C@H](C)C[C@H]2C(=O)Nc2nc(Br)ccc2C)c2ccc(-c3cnc(C)nc3)cc12]

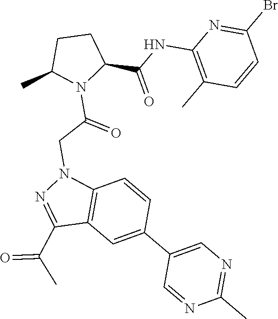 [SMILES: CC(=O)c1nn(CC(=O)N2[C@@H](C)CC[C@H]2C(=O)Nc2nc(Br)ccc2C)c2ccc(-c3cnc(C)nc3)cc12]

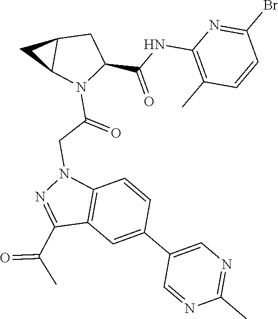 [SMILES: CC(=O)c1nn(CC(=O)N2[C@H](C(=O)Nc3nc(Br)ccc3C)C[C@@H]3C[C@@H]32)c2ccc(-c3cnc(C)nc3)cc12]

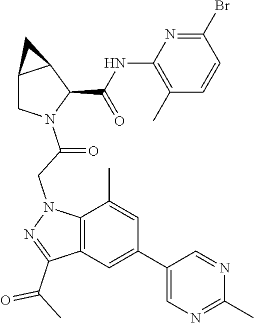 [SMILES: CC(=O)c1nn(CC(=O)N2C[C@H]3C[C@H]3[C@H]2C(=O)Nc2nc(Br)ccc2C)c2c(C)cc(-c3cnc(C)nc3)cc12]

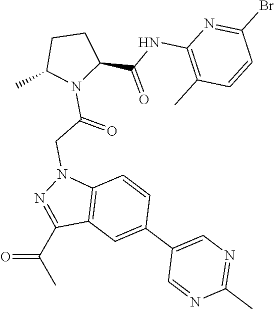 [SMILES: CC(=O)c1nn(CC(=O)N2[C@H](C)CC[C@H]2C(=O)Nc2nc(Br)ccc2C)c2ccc(-c3cnc(C)nc3)cc12]

In some embodiments, the compound of formula (V) is:

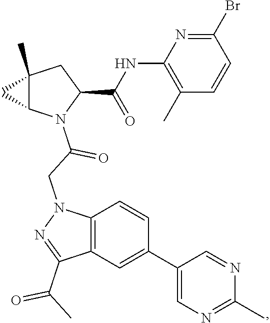 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

or a pharmaceutically acceptable salt thereof.

In some embodiments, the compound of formula (V) is:

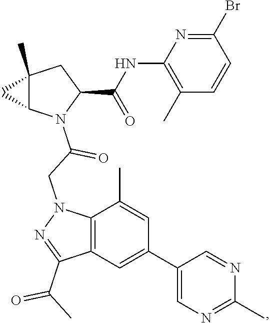 [SMILES: CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]

or a pharmaceutically acceptable salt thereof.

In some embodiments, the compound of formula (V) is a compound disclosed in WO2018/160889A1. For example, the compound of formula (V) is a compound of Table 2 or a pharmaceutically acceptable salt thereof.

TABLE 2

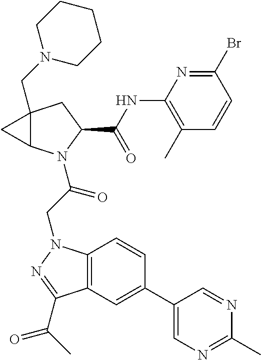 [SMILES: CC(=O)c1nn(CC(=O)N2C3CC3(CN3CCCCC3)C[C@H]2C(=O)Nc2nc(Br)ccc2C)c2ccc(-c3cnc(C)nc3)cc12]

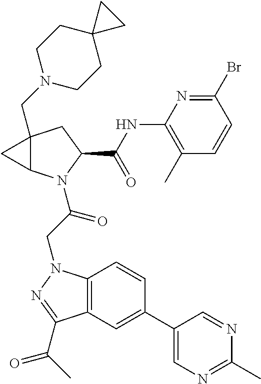 [SMILES: CC(=O)c1nn(CC(=O)N2C3CC3(CN3CCC4(CC3)CC4)C[C@H]2C(=O)Nc2nc(Br)ccc2C)c2ccc(-c3cnc(C)nc3)cc12]

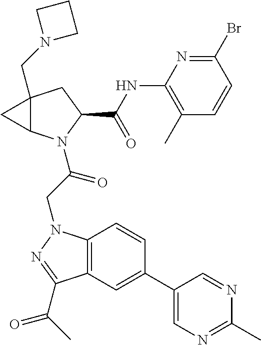 [SMILES: CC(=O)c1nn(CC(=O)N2C3CC3(CN3CCC3)C[C@H]2C(=O)Nc2nc(Br)ccc2C)c2ccc(-c3cnc(C)nc3)cc12]

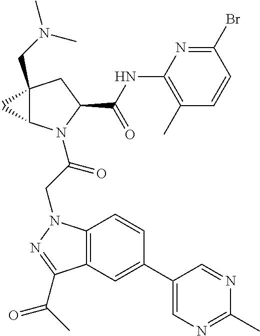 [SMILES: CC(=O)c1nn(CC(=O)N2[C@H](C(=O)Nc3nc(Br)ccc3C)C[C@@]3(CN(C)C)C[C@@H]23)c2ccc(-c3cnc(C)nc3)cc12]

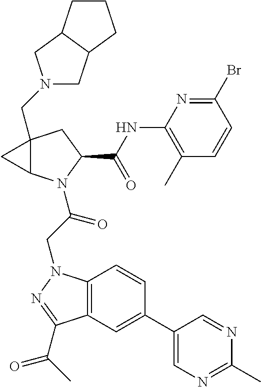 [SMILES: CC(=O)c1nn(CC(=O)N2C3CC3(CN3CC4CCCC4C3)C[C@H]2C(=O)Nc2nc(Br)ccc2C)c2ccc(-c3cnc(C)nc3)cc12]

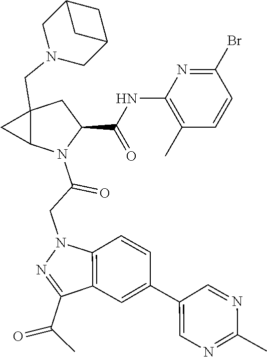 [SMILES: CC(=O)c1nn(CC(=O)N2C3CC3(CN3CC4CC(C4)C3)C[C@H]2C(=O)Nc2nc(Br)ccc2C)c2ccc(-c3cnc(C)nc3)cc12]

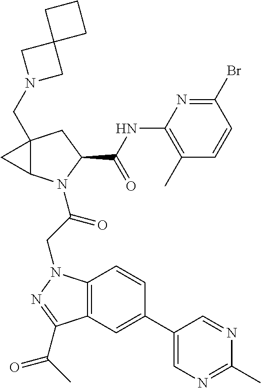 [SMILES: CC(=O)c1nn(CC(=O)N2C3CC3(CN3CC4(CCC4)C3)C[C@H]2C(=O)Nc2nc(Br)ccc2C)c2ccc(-c3cnc(C)nc3)cc12]

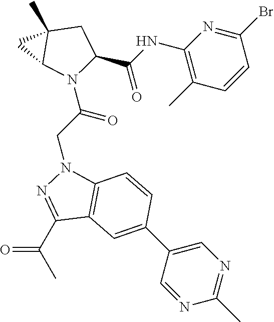 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

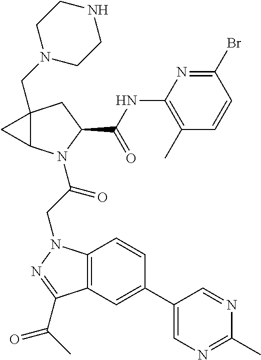 [SMILES: CC(=O)c1nn(CC(=O)N2C3CC3(CN3CCNCC3)C[C@H]2C(=O)Nc2nc(Br)ccc2C)c2ccc(-c3cnc(C)nc3)cc12]

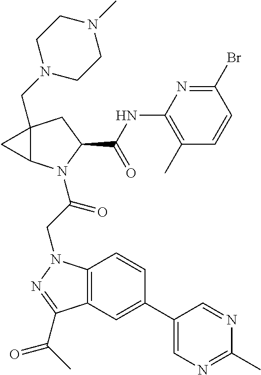 [SMILES: CC(=O)c1nn(CC(=O)N2C3CC3(CN3CCN(C)CC3)C[C@H]2C(=O)Nc2nc(Br)ccc2C)c2ccc(-c3cnc(C)nc3)cc12]

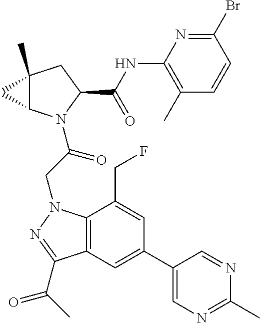 [SMILES: CC(=O)c1nn(CC(=O)N2[C@H](C(=O)Nc3nc(Br)ccc3C)C[C@@]3(C)C[C@@H]23)c2c(CF)cc(-c3cnc(C)nc3)cc12]

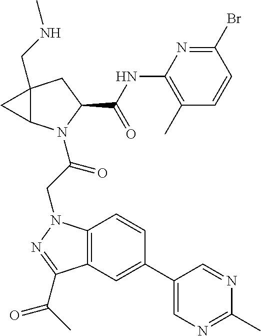 [SMILES: CNCC12CC1N(C(=O)Cn1nc(C(C)=O)c3cc(-c4cnc(C)nc4)ccc31)[C@H](C(=O)Nc1nc(Br)ccc1C)C2]

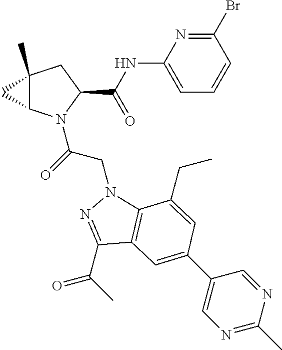

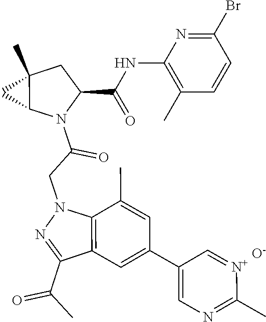

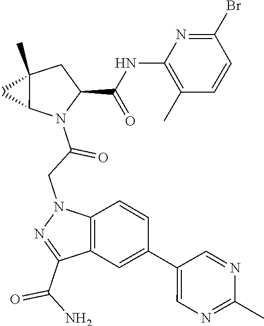 [SMILES: Cc1ncc(-c2ccc3c(c2)c(C(N)=O)nn3CC(=O)N2[C@H](C(=O)Nc3nc(Br)ccc3C)C[C@@]3(C)C[C@@H]23)cn1]

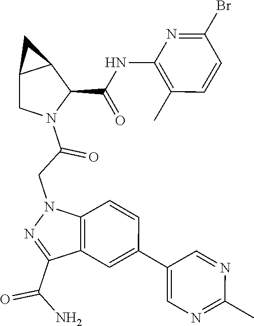 [SMILES: Cc1ncc(-c2ccc3c(c2)c(C(N)=O)nn3CC(=O)N2C[C@H]3C[C@H]3[C@H]2C(=O)Nc2nc(Br)ccc2C)cn1]

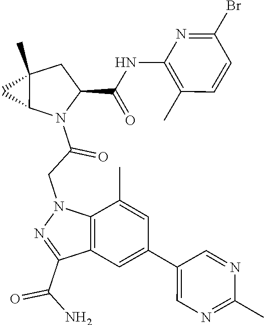 [SMILES: Cc1ncc(-c2cc(C)c3c(c2)c(C(N)=O)nn3CC(=O)N2[C@H](C(=O)Nc3nc(Br)ccc3C)C[C@@]3(C)C[C@@H]23)cn1]

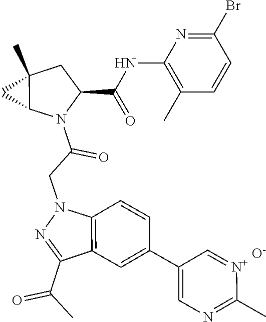

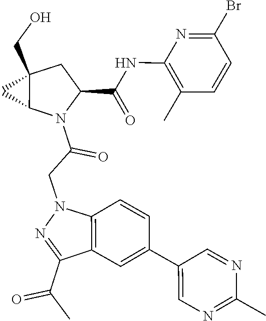 [SMILES: CC(=O)c1nn(CC(=O)N2[C@H](C(=O)Nc3nc(Br)ccc3C)C[C@@]3(CO)C[C@@H]23)c2ccc(-c3cnc(C)nc3)cc12]

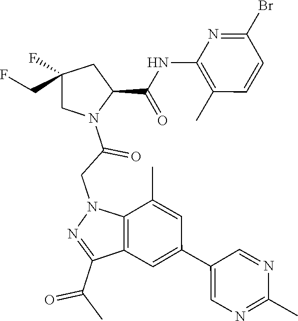 [SMILES: CC(=O)c1nn(CC(=O)N2C[C@@](F)(CF)C[C@H]2C(=O)Nc2nc(Br)ccc2C)c2c(C)cc(-c3cnc(C)nc3)cc12]

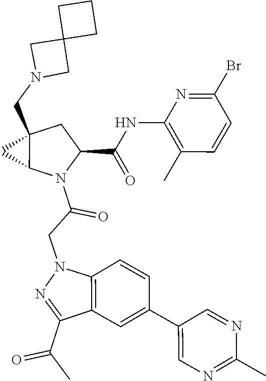 [SMILES: CC(=O)c1nn(CC(=O)N2[C@H](C(=O)Nc3nc(Br)ccc3C)C[C@@]3(CN4CC5(CCC5)C4)C[C@@H]23)c2ccc(-c3cnc(C)nc3)cc12]

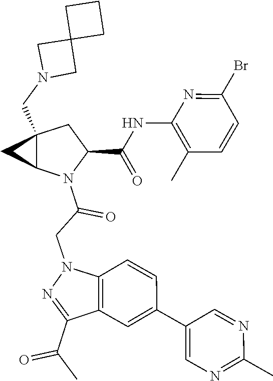 [SMILES: CC(=O)c1nn(CC(=O)N2[C@H]3C[C@@]3(CN3CC4(CCC4)C3)C[C@H]2C(=O)Nc2nc(Br)ccc2C)c2ccc(-c3cnc(C)nc3)cc12]

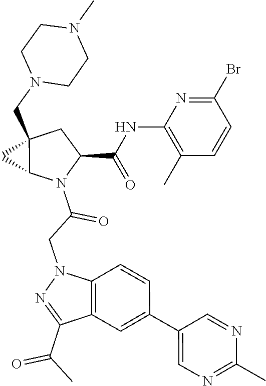 [SMILES: CC(=O)c1nn(CC(=O)N2[C@H](C(=O)Nc3nc(Br)ccc3C)C[C@@]3(CN4CCN(C)CC4)C[C@@H]23)c2ccc(-c3cnc(C)nc3)cc12]

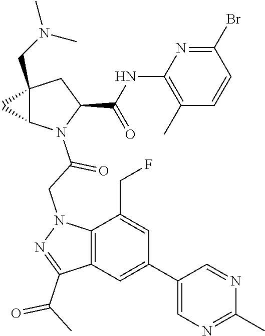 [SMILES: CC(=O)c1nn(CC(=O)N2[C@H](C(=O)Nc3nc(Br)ccc3C)C[C@@]3(CN(C)C)C[C@@H]23)c2c(CF)cc(-c3cnc(C)nc3)cc12]

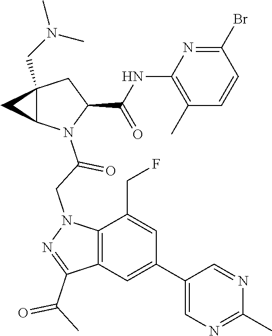 [SMILES: CC(=O)c1nn(CC(=O)N2[C@H]3C[C@@]3(CN(C)C)C[C@H]2C(=O)Nc2nc(Br)ccc2C)c2c(CF)cc(-c3cnc(C)nc3)cc12]

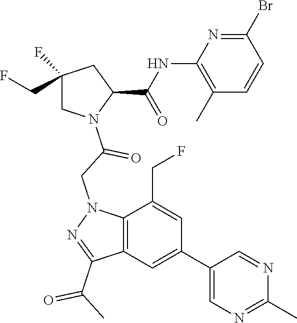 [SMILES: CC(=O)c1nn(CC(=O)N2C[C@@](F)(CF)C[C@H]2C(=O)Nc2nc(Br)ccc2C)c2c(CF)cc(-c3cnc(C)nc3)cc12]

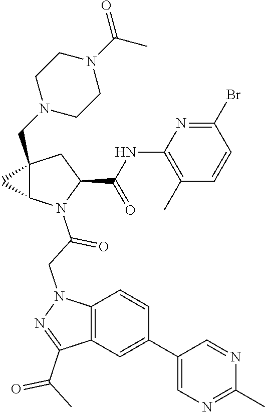 [SMILES: CC(=O)c1nn(CC(=O)N2[C@H](C(=O)Nc3nc(Br)ccc3C)C[C@@]3(CN4CCN(C(C)=O)CC4)C[C@@H]23)c2ccc(-c3cnc(C)nc3)cc12]

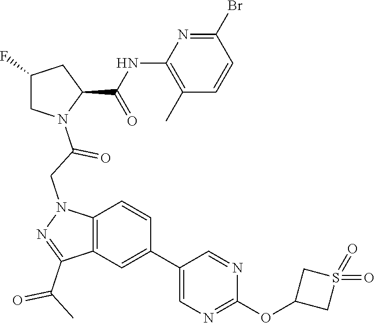 [SMILES: CC(=O)c1nn(CC(=O)N2C[C@H](F)C[C@H]2C(=O)Nc2nc(Br)ccc2C)c2ccc(-c3cnc(OC4CS(=O)(=O)C4)nc3)cc12]

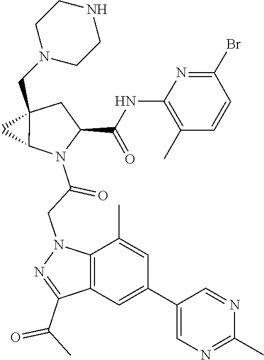 [SMILES: CC(=O)c1nn(CC(=O)N2[C@H](C(=O)Nc3nc(Br)ccc3C)C[C@@]3(CN4CCNCC4)C[C@@H]23)c2c(C)cc(-c3cnc(C)nc3)cc12]

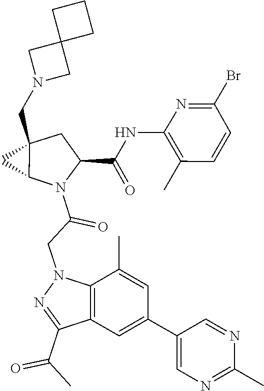 [SMILES: CC(=O)c1nn(CC(=O)N2[C@H](C(=O)Nc3nc(Br)ccc3C)C[C@@]3(CN4CC5(CCC5)C4)C[C@@H]23)c2c(C)cc(-c3cnc(C)nc3)cc12]

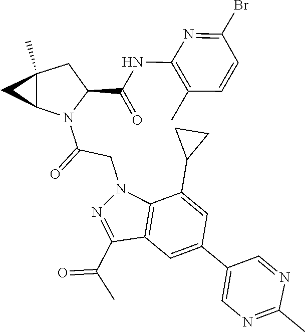 [SMILES: CC(=O)c1nn(CC(=O)N2[C@H]3C[C@@]3(C)C[C@H]2C(=O)Nc2nc(Br)ccc2C)c2c(C3CC3)cc(-c3cnc(C)nc3)cc12]

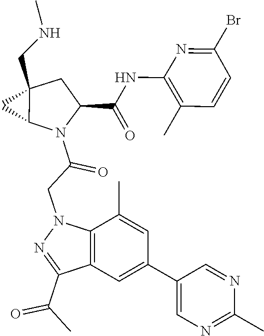 [SMILES: CNC[C@@]12C[C@@H](C(=O)Nc3nc(Br)ccc3C)N(C(=O)Cn3nc(C(C)=O)c4cc(-c5cnc(C)nc5)cc(C)c43)[C@@H]1C2]

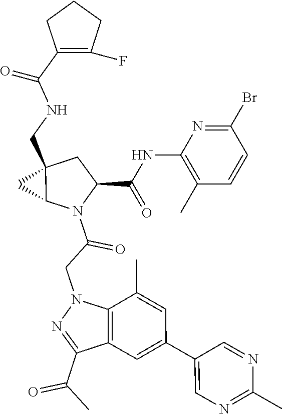 [SMILES: CC(=O)c1nn(CC(=O)N2[C@H](C(=O)Nc3nc(Br)ccc3C)C[C@@]3(CNC(=O)C4=C(F)CCC4)C[C@@H]23)c2c(C)cc(-c3cnc(C)nc3)cc12]

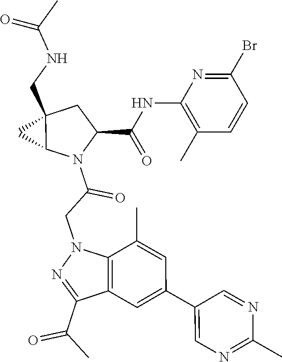 [SMILES: CC(=O)NC[C@@]12C[C@@H](C(=O)Nc3nc(Br)ccc3C)N(C(=O)Cn3nc(C(C)=O)c4cc(-c5cnc(C)nc5)cc(C)c43)[C@@H]1C2]

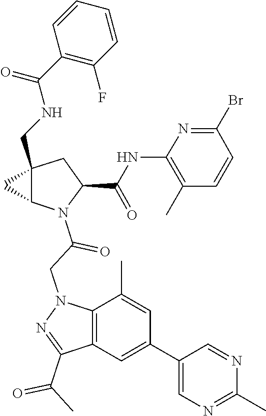 [SMILES: CC(=O)c1nn(CC(=O)N2[C@H](C(=O)Nc3nc(Br)ccc3C)C[C@@]3(CNC(=O)c4ccccc4F)C[C@@H]23)c2c(C)cc(-c3cnc(C)nc3)cc12]

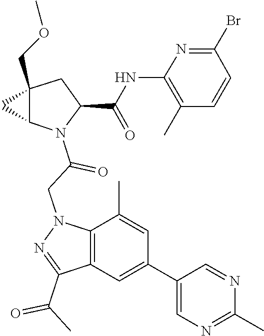 [SMILES: COC[C@@]12C[C@@H](C(=O)Nc3nc(Br)ccc3C)N(C(=O)Cn3nc(C(C)=O)c4cc(-c5cnc(C)nc5)cc(C)c43)[C@@H]1C2]

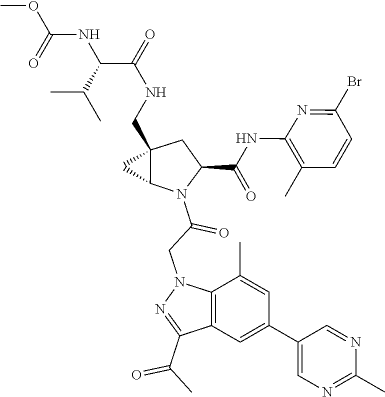

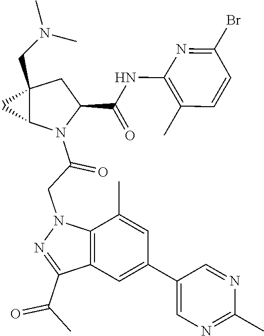 [SMILES: CC(=O)c1nn(CC(=O)N2[C@H](C(=O)Nc3nc(Br)ccc3C)C[C@@]3(CN(C)C)C[C@@H]23)c2c(C)cc(-c3cnc(C)nc3)cc12]

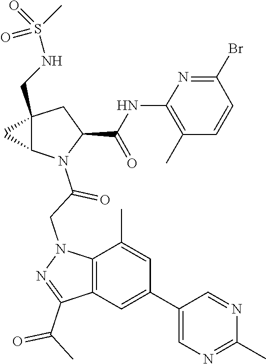 [SMILES: CC(=O)c1nn(CC(=O)N2[C@H](C(=O)Nc3nc(Br)ccc3C)C[C@@]3(CNS(C)(=O)=O)C[C@@H]23)c2c(C)cc(-c3cnc(C)nc3)cc12]

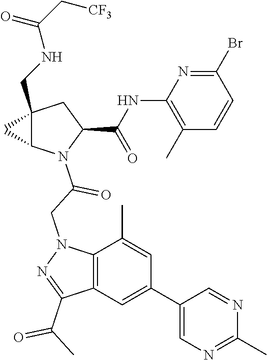 [SMILES: CC(=O)c1nn(CC(=O)N2[C@H](C(=O)Nc3nc(Br)ccc3C)C[C@@]3(CNC(=O)CC(F)(F)F)C[C@@H]23)c2c(C)cc(-c3cnc(C)nc3)cc12]

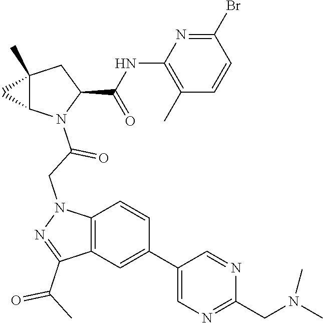 [SMILES: CC(=O)c1nn(CC(=O)N2[C@H](C(=O)Nc3nc(Br)ccc3C)C[C@@]3(C)C[C@@H]23)c2ccc(-c3cnc(CN(C)C)nc3)cc12]

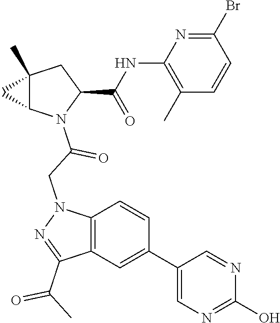 [SMILES: CC(=O)c1nn(CC(=O)N2[C@H](C(=O)Nc3nc(Br)ccc3C)C[C@@]3(C)C[C@@H]23)c2ccc(-c3cnc(O)nc3)cc12]

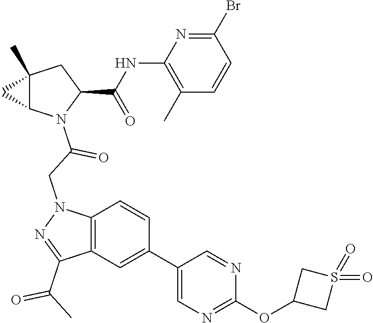 [SMILES: CC(=O)c1nn(CC(=O)N2[C@H](C(=O)Nc3nc(Br)ccc3C)C[C@@]3(C)C[C@@H]23)c2ccc(-c3cnc(OC4CS(=O)(=O)C4)nc3)cc12]

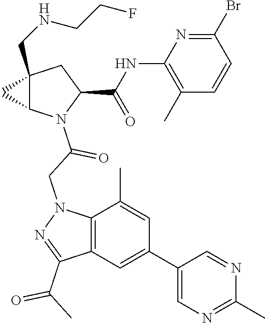 [SMILES: CC(=O)c1nn(CC(=O)N2[C@H](C(=O)Nc3nc(Br)ccc3C)C[C@@]3(CNCCF)C[C@@H]23)c2c(C)cc(-c3cnc(C)nc3)cc12]

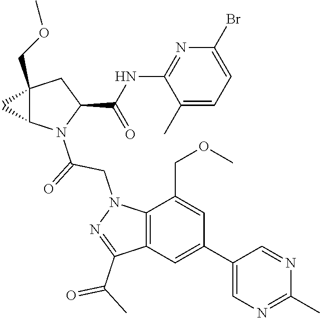 [SMILES: COCc1cc(-c2cnc(C)nc2)cc2c(C(C)=O)nn(CC(=O)N3[C@H](C(=O)Nc4nc(Br)ccc4C)C[C@@]4(COC)C[C@@H]34)c12]

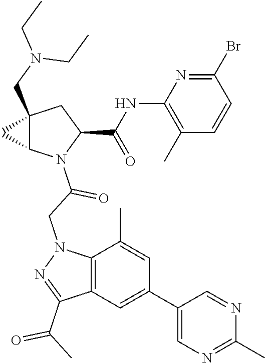 [SMILES: CCN(CC)C[C@@]12C[C@@H](C(=O)Nc3nc(Br)ccc3C)N(C(=O)Cn3nc(C(C)=O)c4cc(-c5cnc(C)nc5)cc(C)c43)[C@@H]1C2]

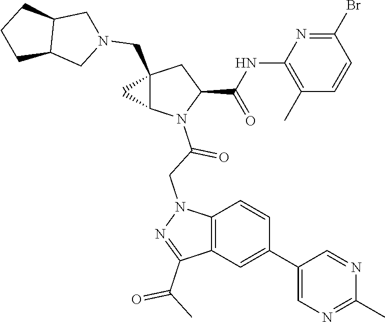 [SMILES: CC(=O)c1nn(CC(=O)N2[C@H](C(=O)Nc3nc(Br)ccc3C)C[C@@]3(CN4C[C@H]5CCC[C@H]5C4)C[C@@H]23)c2ccc(-c3cnc(C)nc3)cc12]

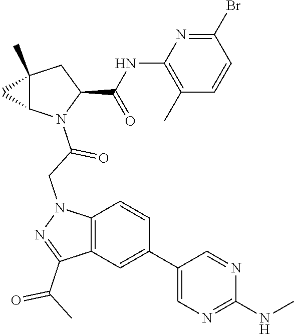 [SMILES: CNc1ncc(-c2ccc3c(c2)c(C(C)=O)nn3CC(=O)N2[C@H](C(=O)Nc3nc(Br)ccc3C)C[C@@]3(C)C[C@@H]23)cn1]

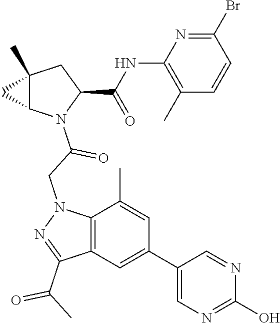 [SMILES: CC(=O)c1nn(CC(=O)N2[C@H](C(=O)Nc3nc(Br)ccc3C)C[C@@]3(C)C[C@@H]23)c2c(C)cc(-c3cnc(O)nc3)cc12]

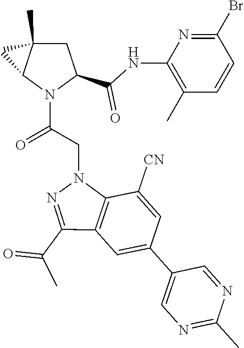 [SMILES: CC(=O)c1nn(CC(=O)N2[C@H](C(=O)Nc3nc(Br)ccc3C)C[C@@]3(C)C[C@@H]23)c2c(C#N)cc(-c3cnc(C)nc3)cc12]

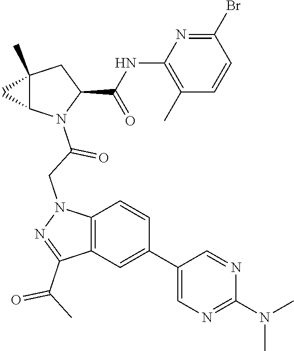 [SMILES: CC(=O)c1nn(CC(=O)N2[C@H](C(=O)Nc3nc(Br)ccc3C)C[C@@]3(C)C[C@@H]23)c2ccc(-c3cnc(N(C)C)nc3)cc12]

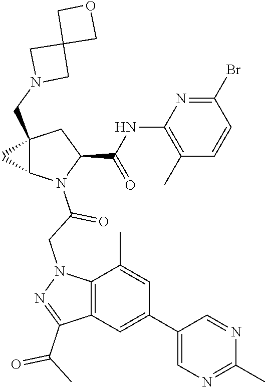 [SMILES: CC(=O)c1nn(CC(=O)N2[C@H](C(=O)Nc3nc(Br)ccc3C)C[C@@]3(CN4CC5(COC5)C4)C[C@@H]23)c2c(C)cc(-c3cnc(C)nc3)cc12]

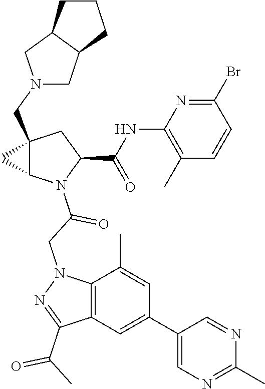 [SMILES: CC(=O)c1nn(CC(=O)N2[C@H](C(=O)Nc3nc(Br)ccc3C)C[C@@]3(CN4C[C@H]5CCC[C@H]5C4)C[C@@H]23)c2c(C)cc(-c3cnc(C)nc3)cc12]

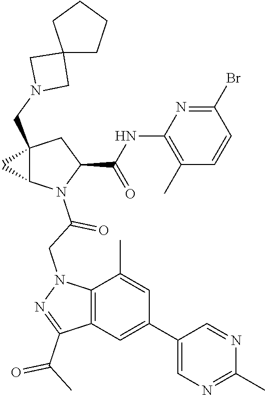 [SMILES: CC(=O)c1nn(CC(=O)N2[C@H](C(=O)Nc3nc(Br)ccc3C)C[C@@]3(CN4CC5(CCCC5)C4)C[C@@H]23)c2c(C)cc(-c3cnc(C)nc3)cc12]

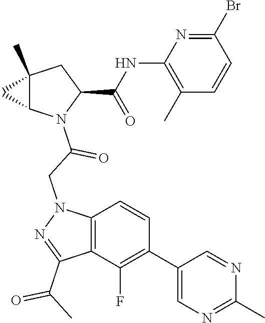 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)c(F)c12]

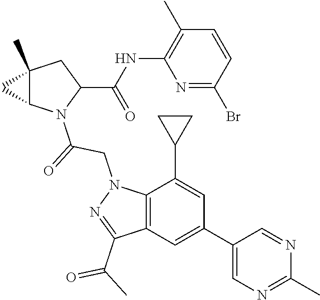 [SMILES: CC(=O)c1nn(CC(=O)N2C(C(=O)Nc3nc(Br)ccc3C)C[C@@]3(C)C[C@@H]23)c2c(C3CC3)cc(-c3cnc(C)nc3)cc12]

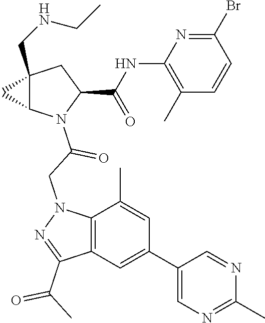 [SMILES: CCNC[C@@]12C[C@@H](C(=O)Nc3nc(Br)ccc3C)N(C(=O)Cn3nc(C(C)=O)c4cc(-c5cnc(C)nc5)cc(C)c43)[C@@H]1C2]

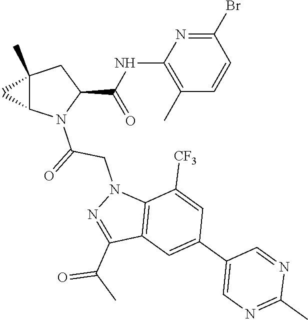 [SMILES: CC(=O)c1nn(CC(=O)N2[C@H](C(=O)Nc3nc(Br)ccc3C)C[C@@]3(C)C[C@@H]23)c2c(C(F)(F)F)cc(-c3cnc(C)nc3)cc12]

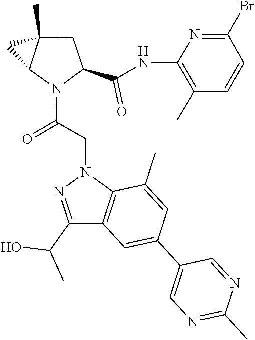 [SMILES: Cc1ncc(-c2cc(C)c3c(c2)c(C(C)O)nn3CC(=O)N2[C@H](C(=O)Nc3nc(Br)ccc3C)C[C@@]3(C)C[C@@H]23)cn1]

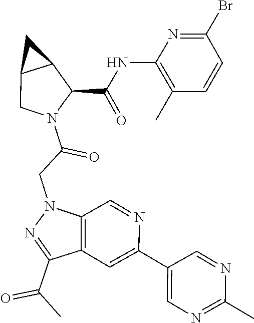 [SMILES: CC(=O)c1nn(CC(=O)N2C[C@H]3C[C@H]3[C@H]2C(=O)Nc2nc(Br)ccc2C)c2cnc(-c3cnc(C)nc3)cc12]

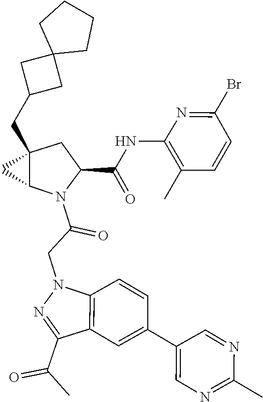 [SMILES: CC(=O)c1nn(CC(=O)N2[C@H](C(=O)Nc3nc(Br)ccc3C)C[C@@]3(CC4CC5(CCCC5)C4)C[C@@H]23)c2ccc(-c3cnc(C)nc3)cc12]

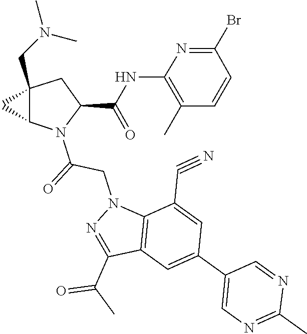 [SMILES: CC(=O)c1nn(CC(=O)N2[C@H](C(=O)Nc3nc(Br)ccc3C)C[C@@]3(CN(C)C)C[C@@H]23)c2c(C#N)cc(-c3cnc(C)nc3)cc12]

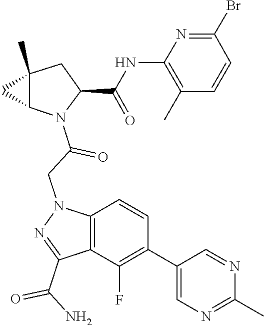 [SMILES: Cc1ncc(-c2ccc3c(c(C(N)=O)nn3CC(=O)N3[C@H](C(=O)Nc4nc(Br)ccc4C)C[C@@]4(C)C[C@@H]34)c2F)cn1]

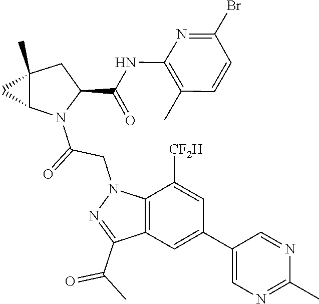 [SMILES: CC(=O)c1nn(CC(=O)N2[C@H](C(=O)Nc3nc(Br)ccc3C)C[C@@]3(C)C[C@@H]23)c2c(C(F)F)cc(-c3cnc(C)nc3)cc12]

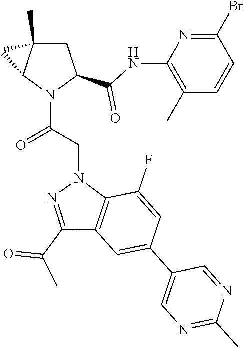 [SMILES: CC(=O)c1nn(CC(=O)N2[C@H](C(=O)Nc3nc(Br)ccc3C)C[C@@]3(C)C[C@@H]23)c2c(F)cc(-c3cnc(C)nc3)cc12]

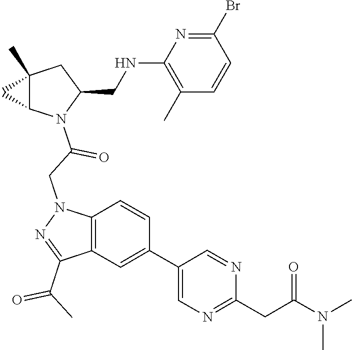 [SMILES: CC(=O)c1nn(CC(=O)N2[C@H](CNc3nc(Br)ccc3C)C[C@@]3(C)C[C@@H]23)c2ccc(-c3cnc(CC(=O)N(C)C)nc3)cc12]

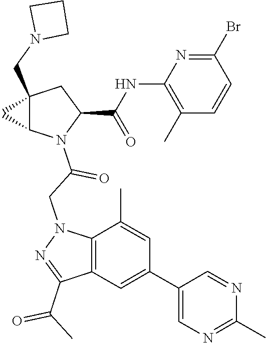 [SMILES: CC(=O)c1nn(CC(=O)N2[C@H](C(=O)Nc3nc(Br)ccc3C)C[C@@]3(CN4CCC4)C[C@@H]23)c2c(C)cc(-c3cnc(C)nc3)cc12]

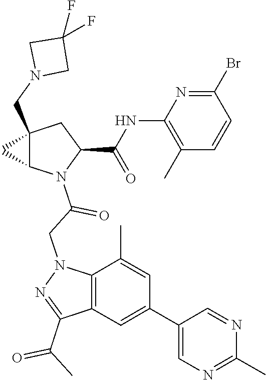 [SMILES: CC(=O)c1nn(CC(=O)N2[C@H](C(=O)Nc3nc(Br)ccc3C)C[C@@]3(CN4CC(F)(F)C4)C[C@@H]23)c2c(C)cc(-c3cnc(C)nc3)cc12]

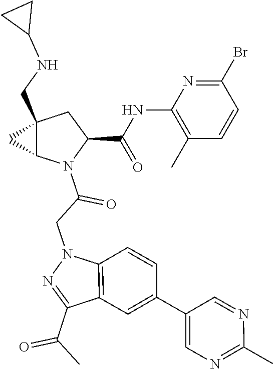 [SMILES: CC(=O)c1nn(CC(=O)N2[C@H](C(=O)Nc3nc(Br)ccc3C)C[C@@]3(CNC4CC4)C[C@@H]23)c2ccc(-c3cnc(C)nc3)cc12]

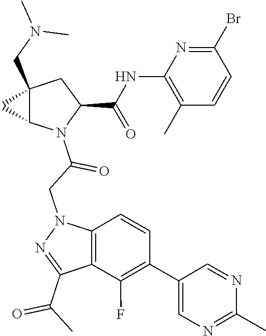 [SMILES: CC(=O)c1nn(CC(=O)N2[C@H](C(=O)Nc3nc(Br)ccc3C)C[C@@]3(CN(C)C)C[C@@H]23)c2ccc(-c3cnc(C)nc3)c(F)c12]

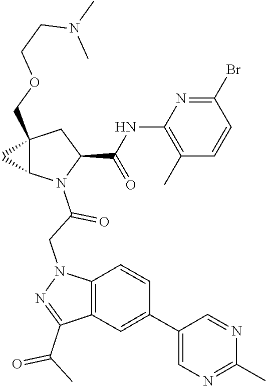 [SMILES: CC(=O)c1nn(CC(=O)N2[C@H](C(=O)Nc3nc(Br)ccc3C)C[C@@]3(COCCN(C)C)C[C@@H]23)c2ccc(-c3cnc(C)nc3)cc12]

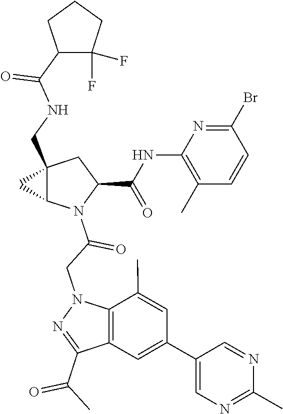 [SMILES: CC(=O)c1nn(CC(=O)N2[C@H](C(=O)Nc3nc(Br)ccc3C)C[C@@]3(CNC(=O)C4CCCC4(F)F)C[C@@H]23)c2c(C)cc(-c3cnc(C)nc3)cc12]

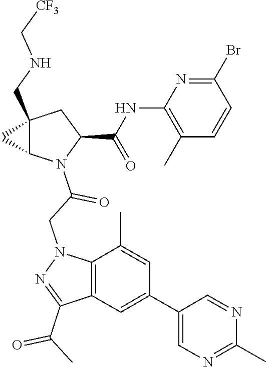 [SMILES: CC(=O)c1nn(CC(=O)N2[C@H](C(=O)Nc3nc(Br)ccc3C)C[C@@]3(CNCC(F)(F)F)C[C@@H]23)c2c(C)cc(-c3cnc(C)nc3)cc12]

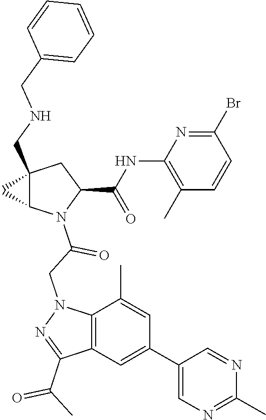 [SMILES: CC(=O)c1nn(CC(=O)N2[C@H](C(=O)Nc3nc(Br)ccc3C)C[C@@]3(CNCc4ccccc4)C[C@@H]23)c2c(C)cc(-c3cnc(C)nc3)cc12]

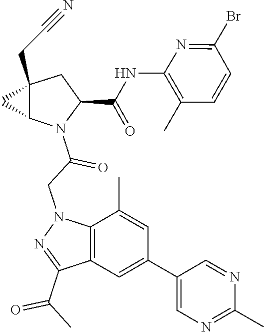 [SMILES: CC(=O)c1nn(CC(=O)N2[C@H](C(=O)Nc3nc(Br)ccc3C)C[C@@]3(CC#N)C[C@@H]23)c2c(C)cc(-c3cnc(C)nc3)cc12]

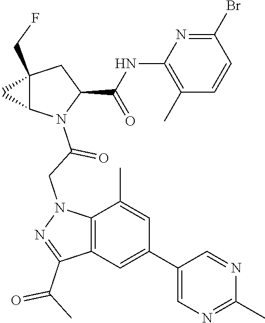 [SMILES: CC(=O)c1nn(CC(=O)N2[C@H](C(=O)Nc3nc(Br)ccc3C)C[C@@]3(CF)C[C@@H]23)c2c(C)cc(-c3cnc(C)nc3)cc12]

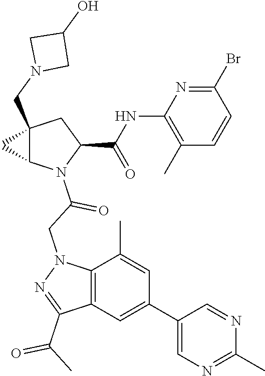 [SMILES: CC(=O)c1nn(CC(=O)N2[C@H](C(=O)Nc3nc(Br)ccc3C)C[C@@]3(CN4CC(O)C4)C[C@@H]23)c2c(C)cc(-c3cnc(C)nc3)cc12]

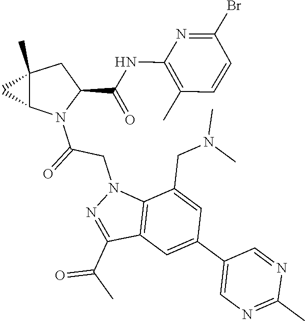 [SMILES: CC(=O)c1nn(CC(=O)N2[C@H](C(=O)Nc3nc(Br)ccc3C)C[C@@]3(C)C[C@@H]23)c2c(CN(C)C)cc(-c3cnc(C)nc3)cc12]

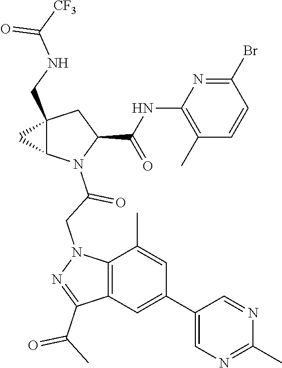 [SMILES: CC(=O)c1nn(CC(=O)N2[C@H](C(=O)Nc3nc(Br)ccc3C)C[C@@]3(CNC(=O)C(F)(F)F)C[C@@H]23)c2c(C)cc(-c3cnc(C)nc3)cc12]

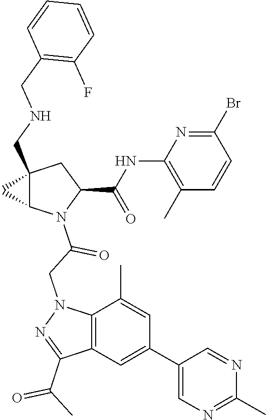 [SMILES: CC(=O)c1nn(CC(=O)N2[C@H](C(=O)Nc3nc(Br)ccc3C)C[C@@]3(CNCc4ccccc4F)C[C@@H]23)c2c(C)cc(-c3cnc(C)nc3)cc12]

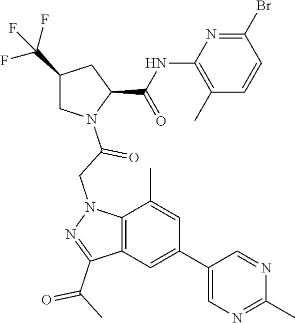 [SMILES: CC(=O)c1nn(CC(=O)N2C[C@@H](C(F)(F)F)C[C@H]2C(=O)Nc2nc(Br)ccc2C)c2c(C)cc(-c3cnc(C)nc3)cc12]

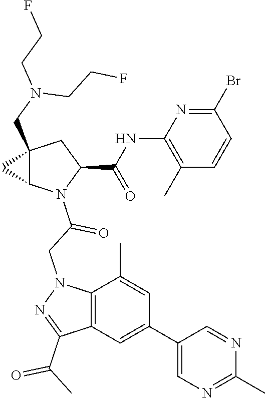 [SMILES: CC(=O)c1nn(CC(=O)N2[C@H](C(=O)Nc3nc(Br)ccc3C)C[C@@]3(CN(CCF)CCF)C[C@@H]23)c2c(C)cc(-c3cnc(C)nc3)cc12]

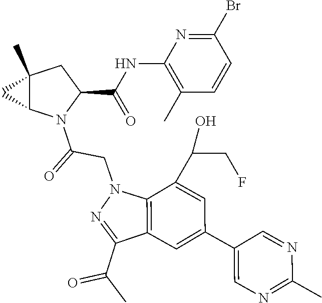 [SMILES: CC(=O)c1nn(CC(=O)N2[C@H](C(=O)Nc3nc(Br)ccc3C)C[C@@]3(C)C[C@@H]23)c2c(C(O)CF)cc(-c3cnc(C)nc3)cc12]

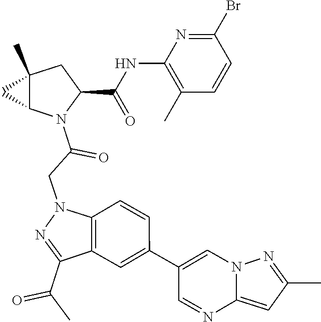 [SMILES: CC(=O)c1nn(CC(=O)N2[C@H](C(=O)Nc3nc(Br)ccc3C)C[C@@]3(C)C[C@@H]23)c2ccc(-c3cnc4cc(C)nn4c3)cc12]

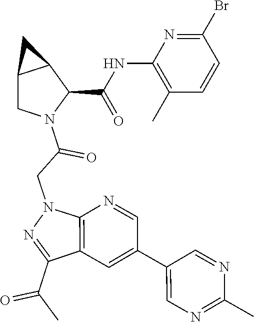 [SMILES: CC(=O)c1nn(CC(=O)N2C[C@H]3C[C@H]3[C@H]2C(=O)Nc2nc(Br)ccc2C)c2ncc(-c3cnc(C)nc3)cc12]

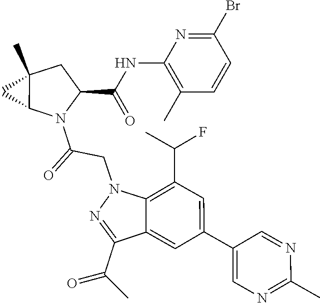 [SMILES: CC(=O)c1nn(CC(=O)N2[C@H](C(=O)Nc3nc(Br)ccc3C)C[C@@]3(C)C[C@@H]23)c2c(C(C)F)cc(-c3cnc(C)nc3)cc12]

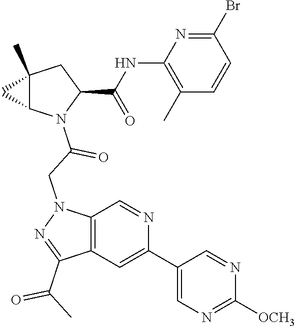 [SMILES: COc1ncc(-c2cc3c(C(C)=O)nn(CC(=O)N4[C@H](C(=O)Nc5nc(Br)ccc5C)C[C@@]5(C)C[C@@H]45)c3cn2)cn1]

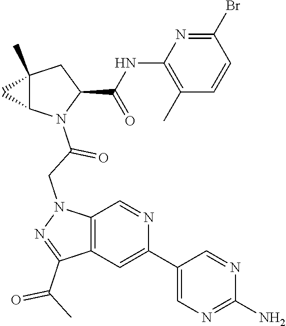 [SMILES: CC(=O)c1nn(CC(=O)N2[C@H](C(=O)Nc3nc(Br)ccc3C)C[C@@]3(C)C[C@@H]23)c2cnc(-c3cnc(N)nc3)cc12]

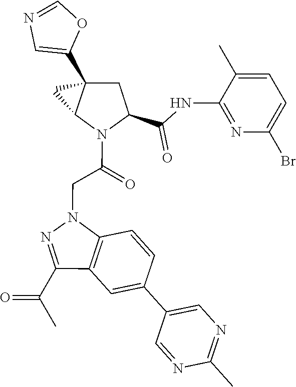 [SMILES: CC(=O)c1nn(CC(=O)N2[C@H](C(=O)Nc3nc(Br)ccc3C)C[C@@]3(c4cnco4)C[C@@H]23)c2ccc(-c3cnc(C)nc3)cc12]

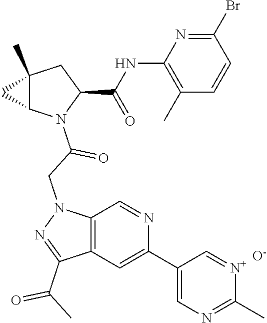

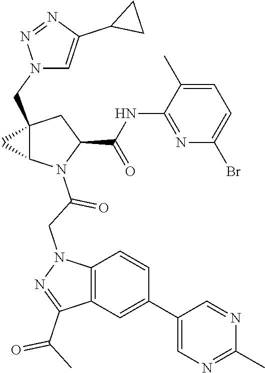 [SMILES: CC(=O)c1nn(CC(=O)N2[C@H](C(=O)Nc3nc(Br)ccc3C)C[C@@]3(Cn4cc(C5CC5)nn4)C[C@@H]23)c2ccc(-c3cnc(C)nc3)cc12]

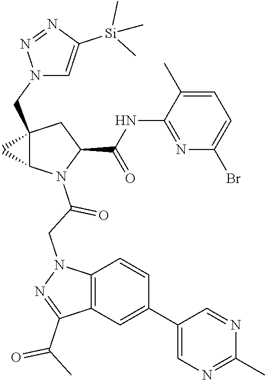 [SMILES: CC(=O)c1nn(CC(=O)N2[C@H](C(=O)Nc3nc(Br)ccc3C)C[C@@]3(Cn4cc([Si](C)(C)C)nn4)C[C@@H]23)c2ccc(-c3cnc(C)nc3)cc12]

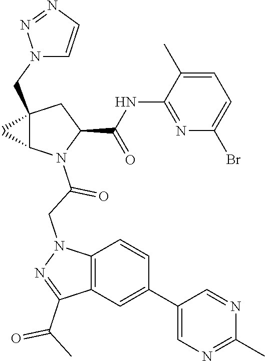 [SMILES: CC(=O)c1nn(CC(=O)N2[C@H](C(=O)Nc3nc(Br)ccc3C)C[C@@]3(Cn4ccnn4)C[C@@H]23)c2ccc(-c3cnc(C)nc3)cc12]

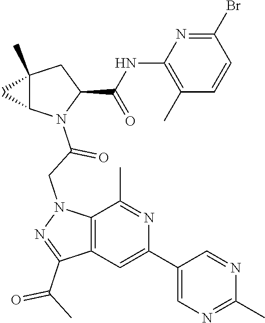 [SMILES: CC(=O)c1nn(CC(=O)N2[C@H](C(=O)Nc3nc(Br)ccc3C)C[C@@]3(C)C[C@@H]23)c2c(C)nc(-c3cnc(C)nc3)cc12]

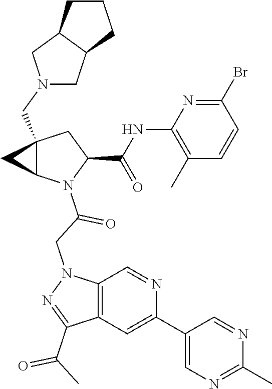 [SMILES: CC(=O)c1nn(CC(=O)N2[C@H]3C[C@@]3(CN3C[C@H]4CCC[C@H]4C3)C[C@H]2C(=O)Nc2nc(Br)ccc2C)c2cnc(-c3cnc(C)nc3)cc12]

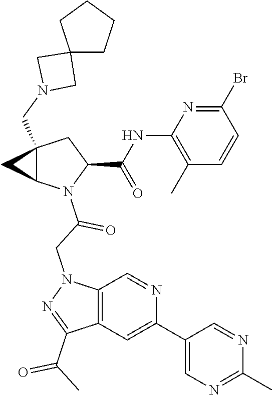 [SMILES: CC(=O)c1nn(CC(=O)N2[C@H]3C[C@@]3(CN3CC4(CCCC4)C3)C[C@H]2C(=O)Nc2nc(Br)ccc2C)c2cnc(-c3cnc(C)nc3)cc12]

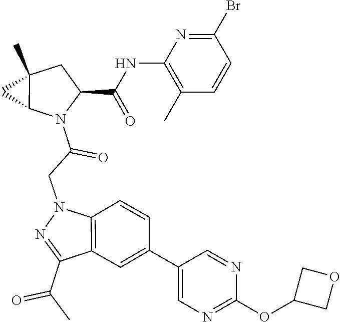 [SMILES: CC(=O)c1nn(CC(=O)N2[C@H](C(=O)Nc3nc(Br)ccc3C)C[C@@]3(C)C[C@@H]23)c2ccc(-c3cnc(OC4COC4)nc3)cc12]

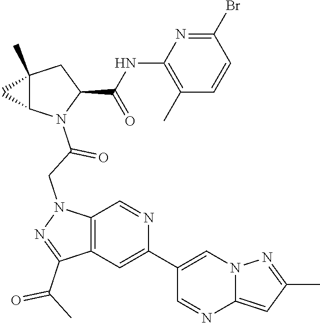 [SMILES: CC(=O)c1nn(CC(=O)N2[C@H](C(=O)Nc3nc(Br)ccc3C)C[C@@]3(C)C[C@@H]23)c2cnc(-c3cnc4cc(C)nn4c3)cc12]

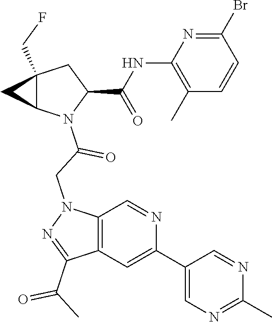 [SMILES: CC(=O)c1nn(CC(=O)N2[C@H]3C[C@@]3(CF)C[C@H]2C(=O)Nc2nc(Br)ccc2C)c2cnc(-c3cnc(C)nc3)cc12]

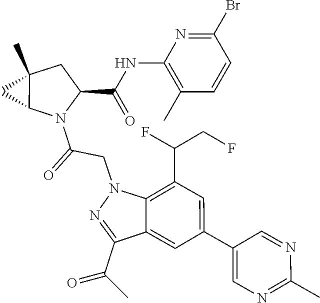 [SMILES: CC(=O)c1nn(CC(=O)N2[C@H](C(=O)Nc3nc(Br)ccc3C)C[C@@]3(C)C[C@@H]23)c2c(C(F)CF)cc(-c3cnc(C)nc3)cc12]

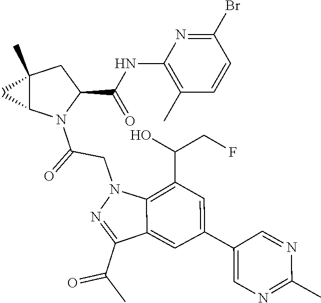 [SMILES: CC(=O)c1nn(CC(=O)N2[C@H](C(=O)Nc3nc(Br)ccc3C)C[C@@]3(C)C[C@@H]23)c2c(C(O)CF)cc(-c3cnc(C)nc3)cc12]

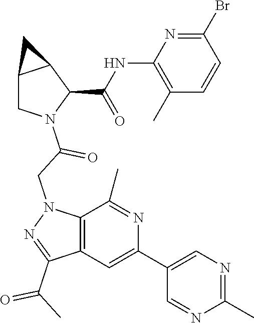 [SMILES: CC(=O)c1nn(CC(=O)N2C[C@H]3C[C@H]3[C@H]2C(=O)Nc2nc(Br)ccc2C)c2c(C)nc(-c3cnc(C)nc3)cc12]

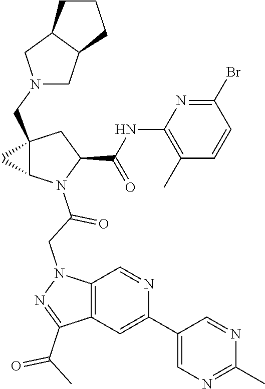 [SMILES: CC(=O)c1nn(CC(=O)N2[C@H](C(=O)Nc3nc(Br)ccc3C)C[C@@]3(CN4C[C@H]5CCC[C@H]5C4)C[C@@H]23)c2cnc(-c3cnc(C)nc3)cc12]

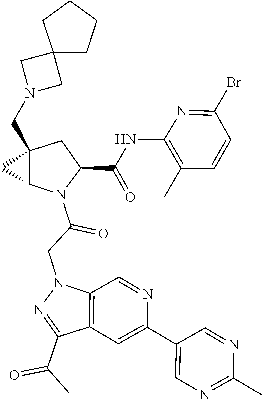 [SMILES: CC(=O)c1nn(CC(=O)N2[C@H](C(=O)Nc3nc(Br)ccc3C)C[C@@]3(CN4CC5(CCCC5)C4)C[C@@H]23)c2cnc(-c3cnc(C)nc3)cc12]

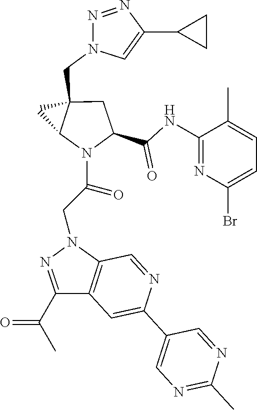 [SMILES: CC(=O)c1nn(CC(=O)N2[C@H](C(=O)Nc3nc(Br)ccc3C)C[C@@]3(Cn4cc(C5CC5)nn4)C[C@@H]23)c2cnc(-c3cnc(C)nc3)cc12]

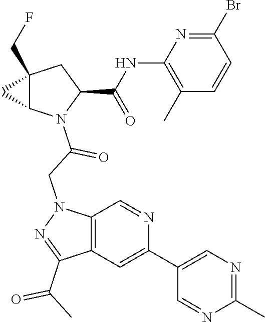 [SMILES: CC(=O)c1nn(CC(=O)N2[C@H](C(=O)Nc3nc(Br)ccc3C)C[C@@]3(CF)C[C@@H]23)c2cnc(-c3cnc(C)nc3)cc12]

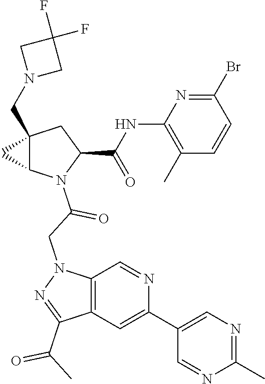 [SMILES: CC(=O)c1nn(CC(=O)N2[C@H](C(=O)Nc3nc(Br)ccc3C)C[C@@]3(CN4CC(F)(F)C4)C[C@@H]23)c2cnc(-c3cnc(C)nc3)cc12]

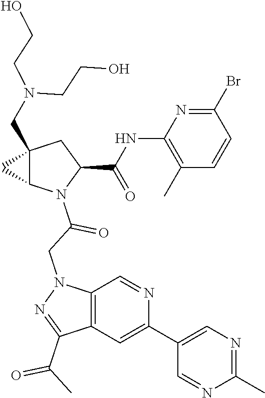 [SMILES: CC(=O)c1nn(CC(=O)N2[C@H](C(=O)Nc3nc(Br)ccc3C)C[C@@]3(CN(CCO)CCO)C[C@@H]23)c2cnc(-c3cnc(C)nc3)cc12]

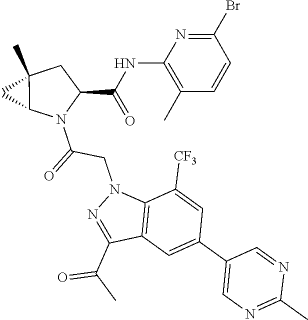 [SMILES: CC(=O)c1nn(CC(=O)N2[C@H](C(=O)Nc3nc(Br)ccc3C)C[C@@]3(C)C[C@@H]23)c2c(C(F)(F)F)cc(-c3cnc(C)nc3)cc12]

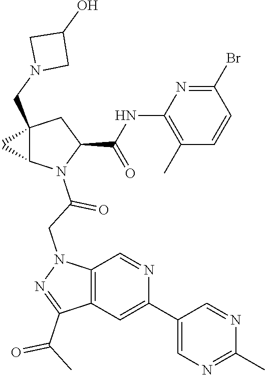 [SMILES: CC(=O)c1nn(CC(=O)N2[C@H](C(=O)Nc3nc(Br)ccc3C)C[C@@]3(CN4CC(O)C4)C[C@@H]23)c2cnc(-c3cnc(C)nc3)cc12]

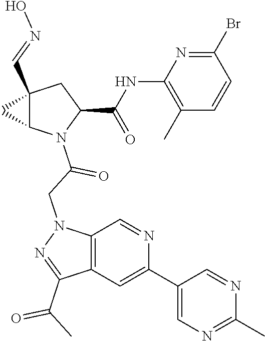 [SMILES: CC(=O)c1nn(CC(=O)N2[C@H](C(=O)Nc3nc(Br)ccc3C)C[C@@]3(/C=N/O)C[C@@H]23)c2cnc(-c3cnc(C)nc3)cc12]

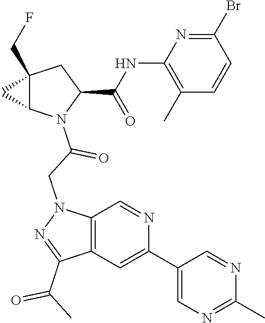 [SMILES: CC(=O)c1nn(CC(=O)N2[C@H](C(=O)Nc3nc(Br)ccc3C)C[C@@]3(CF)C[C@@H]23)c2cnc(-c3cnc(C)nc3)cc12]

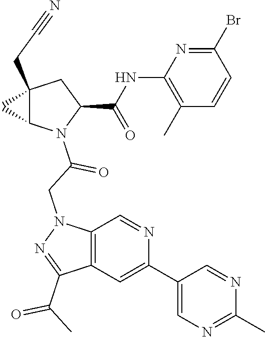 [SMILES: CC(=O)c1nn(CC(=O)N2[C@H](C(=O)Nc3nc(Br)ccc3C)C[C@@]3(CC#N)C[C@@H]23)c2cnc(-c3cnc(C)nc3)cc12]

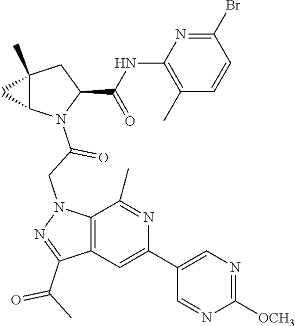 [SMILES: COc1ncc(-c2cc3c(C(C)=O)nn(CC(=O)N4[C@H](C(=O)Nc5nc(Br)ccc5C)C[C@@]5(C)C[C@@H]45)c3c(C)n2)cn1]

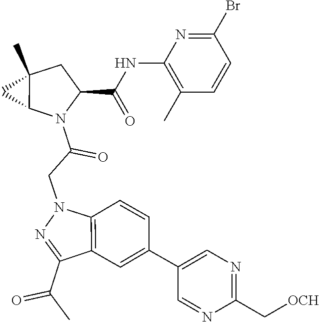

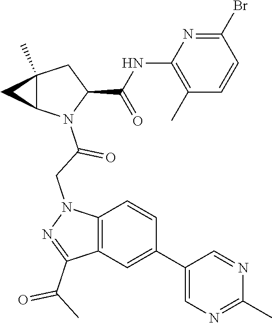 [SMILES: CC(=O)c1nn(CC(=O)N2[C@H]3C[C@@]3(C)C[C@H]2C(=O)Nc2nc(Br)ccc2C)c2ccc(-c3cnc(C)nc3)cc12]

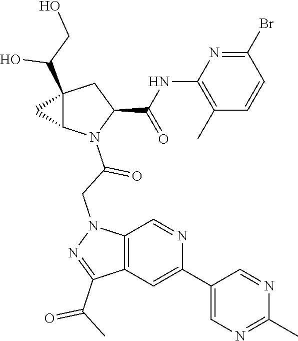 [SMILES: CC(=O)c1nn(CC(=O)N2[C@H](C(=O)Nc3nc(Br)ccc3C)C[C@@]3(C(O)CO)C[C@@H]23)c2cnc(-c3cnc(C)nc3)cc12]

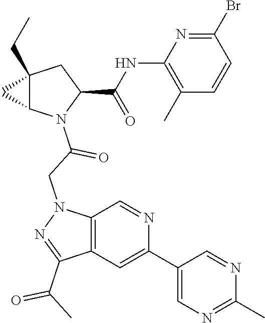 [SMILES: CC[C@@]12C[C@@H](C(=O)Nc3nc(Br)ccc3C)N(C(=O)Cn3nc(C(C)=O)c4cc(-c5cnc(C)nc5)ncc43)[C@@H]1C2]

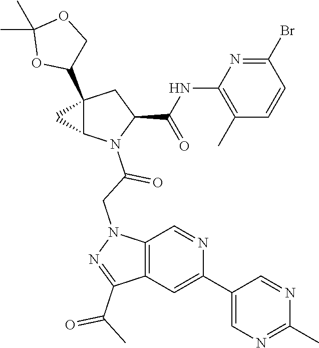

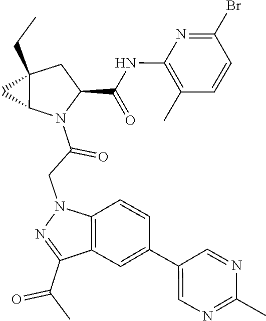 [SMILES: CC[C@@]12C[C@@H](C(=O)Nc3nc(Br)ccc3C)N(C(=O)Cn3nc(C(C)=O)c4cc(-c5cnc(C)nc5)ccc43)[C@@H]1C2]

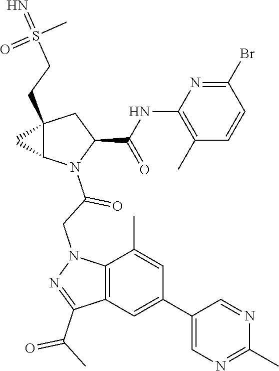 [SMILES: CC(=O)c1nn(CC(=O)N2[C@H](C(=O)Nc3nc(Br)ccc3C)C[C@@]3(CCS(C)(=N)=O)C[C@@H]23)c2c(C)cc(-c3cnc(C)nc3)cc12]

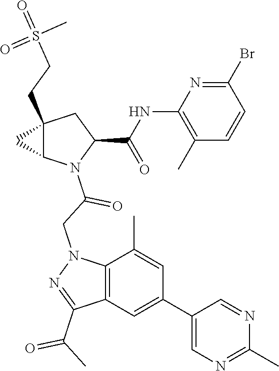 [SMILES: CC(=O)c1nn(CC(=O)N2[C@H](C(=O)Nc3nc(Br)ccc3C)C[C@@]3(CCS(C)(=O)=O)C[C@@H]23)c2c(C)cc(-c3cnc(C)nc3)cc12]

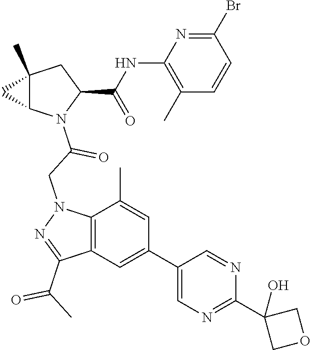 [SMILES: CC(=O)c1nn(CC(=O)N2[C@H](C(=O)Nc3nc(Br)ccc3C)C[C@@]3(C)C[C@@H]23)c2c(C)cc(-c3cnc(C4(O)COC4)nc3)cc12]

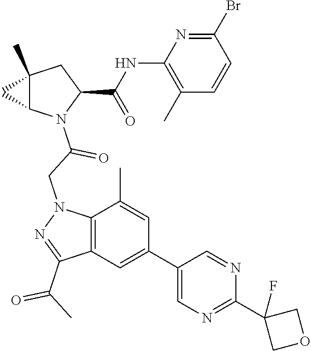 [SMILES: CC(=O)c1nn(CC(=O)N2[C@H](C(=O)Nc3nc(Br)ccc3C)C[C@@]3(C)C[C@@H]23)c2c(C)cc(-c3cnc(C4(F)COC4)nc3)cc12]

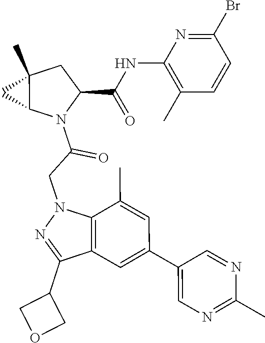 [SMILES: Cc1ncc(-c2cc(C)c3c(c2)c(C2COC2)nn3CC(=O)N2[C@H](C(=O)Nc3nc(Br)ccc3C)C[C@@]3(C)C[C@@H]23)cn1]

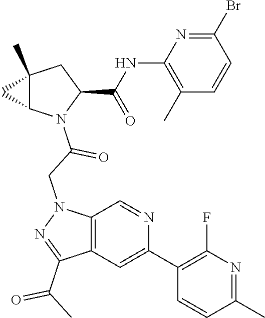 [SMILES: CC(=O)c1nn(CC(=O)N2[C@H](C(=O)Nc3nc(Br)ccc3C)C[C@@]3(C)C[C@@H]23)c2cnc(-c3ccc(C)nc3F)cc12]

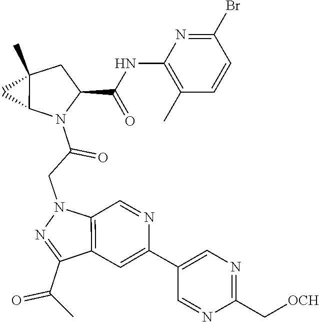

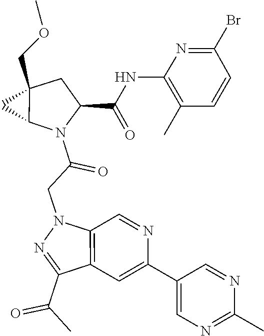 [SMILES: COC[C@@]12C[C@@H](C(=O)Nc3nc(Br)ccc3C)N(C(=O)Cn3nc(C(C)=O)c4cc(-c5cnc(C)nc5)ncc43)[C@@H]1C2]

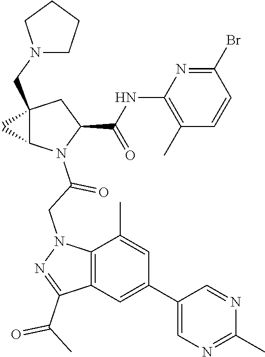 [SMILES: CC(=O)c1nn(CC(=O)N2[C@H](C(=O)Nc3nc(Br)ccc3C)C[C@@]3(CN4CCCC4)C[C@@H]23)c2c(C)cc(-c3cnc(C)nc3)cc12]

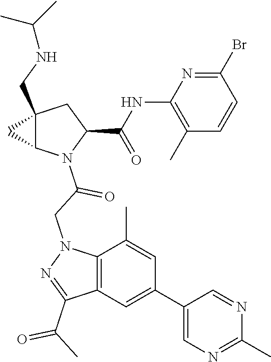 [SMILES: CC(=O)c1nn(CC(=O)N2[C@H](C(=O)Nc3nc(Br)ccc3C)C[C@@]3(CNC(C)C)C[C@@H]23)c2c(C)cc(-c3cnc(C)nc3)cc12]

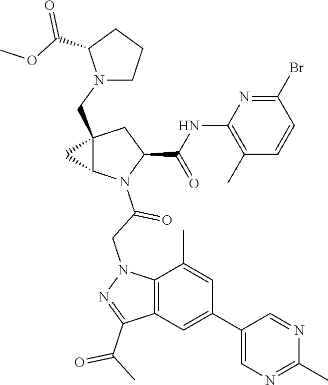

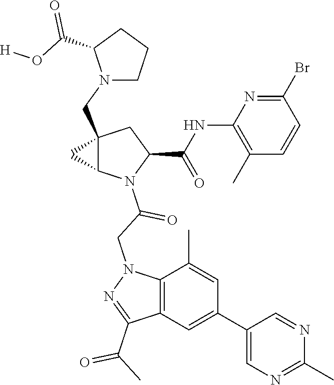 [SMILES: CC(=O)c1nn(CC(=O)N2[C@H](C(=O)Nc3nc(Br)ccc3C)C[C@@]3(CN4CCC[C@H]4C(=O)O)C[C@@H]23)c2c(C)cc(-c3cnc(C)nc3)cc12]

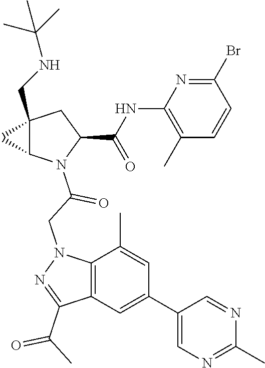 [SMILES: CC(=O)c1nn(CC(=O)N2[C@H](C(=O)Nc3nc(Br)ccc3C)C[C@@]3(CNC(C)(C)C)C[C@@H]23)c2c(C)cc(-c3cnc(C)nc3)cc12]

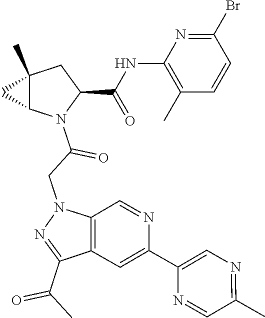 [SMILES: CC(=O)c1nn(CC(=O)N2[C@H](C(=O)Nc3nc(Br)ccc3C)C[C@@]3(C)C[C@@H]23)c2cnc(-c3cnc(C)cn3)cc12]

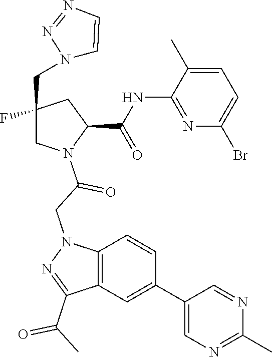 [SMILES: CC(=O)c1nn(CC(=O)N2C[C@@](F)(Cn3ccnn3)C[C@H]2C(=O)Nc2nc(Br)ccc2C)c2ccc(-c3cnc(C)nc3)cc12]

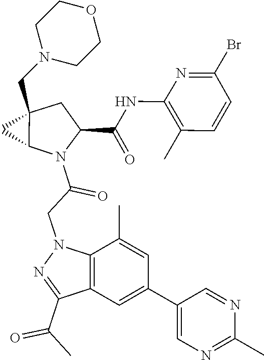 [SMILES: CC(=O)c1nn(CC(=O)N2[C@H](C(=O)Nc3nc(Br)ccc3C)C[C@@]3(CN4CCOCC4)C[C@@H]23)c2c(C)cc(-c3cnc(C)nc3)cc12]

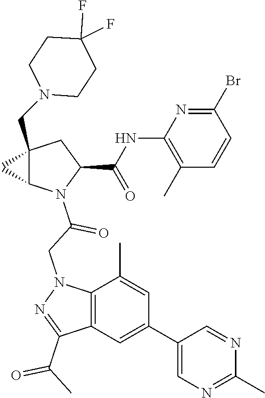 [SMILES: CC(=O)c1nn(CC(=O)N2[C@H](C(=O)Nc3nc(Br)ccc3C)C[C@@]3(CN4CCC(F)(F)CC4)C[C@@H]23)c2c(C)cc(-c3cnc(C)nc3)cc12]

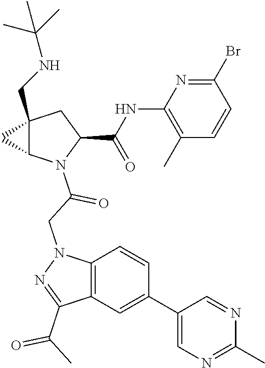 [SMILES: CC(=O)c1nn(CC(=O)N2[C@H](C(=O)Nc3nc(Br)ccc3C)C[C@@]3(CNC(C)(C)C)C[C@@H]23)c2ccc(-c3cnc(C)nc3)cc12]

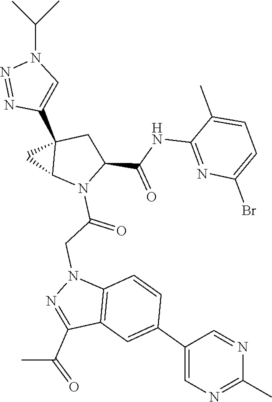 [SMILES: CC(=O)c1nn(CC(=O)N2[C@H](C(=O)Nc3nc(Br)ccc3C)C[C@@]3(c4cn(C(C)C)nn4)C[C@@H]23)c2ccc(-c3cnc(C)nc3)cc12]

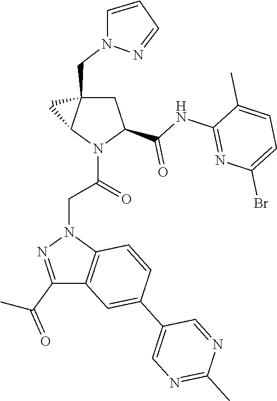 [SMILES: CC(=O)c1nn(CC(=O)N2[C@H](C(=O)Nc3nc(Br)ccc3C)C[C@@]3(Cn4cccn4)C[C@@H]23)c2ccc(-c3cnc(C)nc3)cc12]

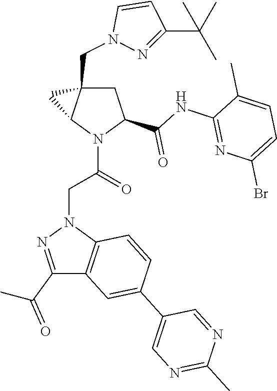 [SMILES: CC(=O)c1nn(CC(=O)N2[C@H](C(=O)Nc3nc(Br)ccc3C)C[C@@]3(Cn4ccc(C(C)(C)C)n4)C[C@@H]23)c2ccc(-c3cnc(C)nc3)cc12]

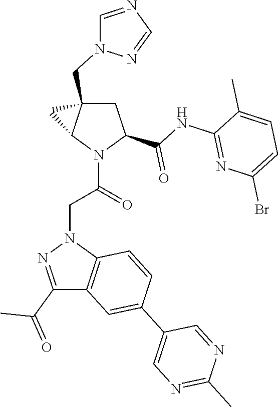 [SMILES: CC(=O)c1nn(CC(=O)N2[C@H](C(=O)Nc3nc(Br)ccc3C)C[C@@]3(Cn4cncn4)C[C@@H]23)c2ccc(-c3cnc(C)nc3)cc12]

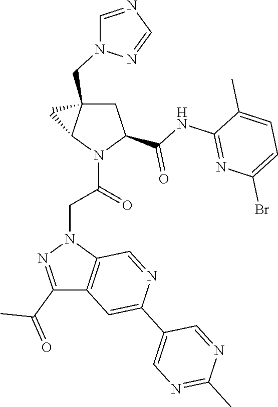 [SMILES: CC(=O)c1nn(CC(=O)N2[C@H](C(=O)Nc3nc(Br)ccc3C)C[C@@]3(Cn4cncn4)C[C@@H]23)c2cnc(-c3cnc(C)nc3)cc12]

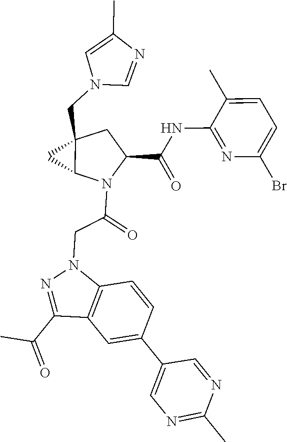 [SMILES: CC(=O)c1nn(CC(=O)N2[C@H](C(=O)Nc3nc(Br)ccc3C)C[C@@]3(Cn4cnc(C)c4)C[C@@H]23)c2ccc(-c3cnc(C)nc3)cc12]

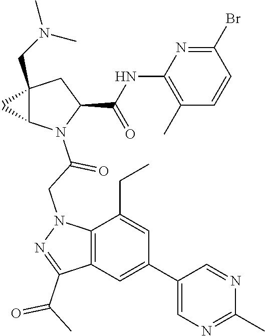 [SMILES: CCc1cc(-c2cnc(C)nc2)cc2c(C(C)=O)nn(CC(=O)N3[C@H](C(=O)Nc4nc(Br)ccc4C)C[C@@]4(CN(C)C)C[C@@H]34)c12]

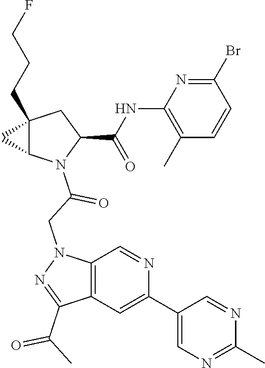 [SMILES: CC(=O)c1nn(CC(=O)N2[C@H](C(=O)Nc3nc(Br)ccc3C)C[C@@]3(CCCF)C[C@@H]23)c2cnc(-c3cnc(C)nc3)cc12]

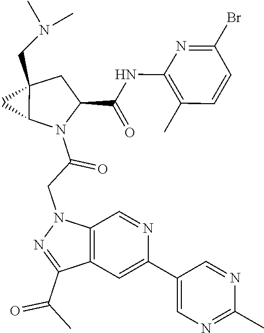 [SMILES: CC(=O)c1nn(CC(=O)N2[C@H](C(=O)Nc3nc(Br)ccc3C)C[C@@]3(CN(C)C)C[C@@H]23)c2cnc(-c3cnc(C)nc3)cc12]

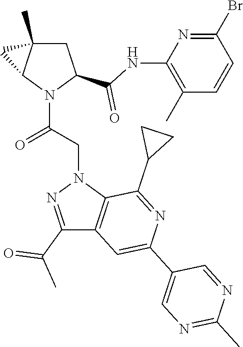 [SMILES: CC(=O)c1nn(CC(=O)N2[C@H](C(=O)Nc3nc(Br)ccc3C)C[C@@]3(C)C[C@@H]23)c2c(C3CC3)nc(-c3cnc(C)nc3)cc12]

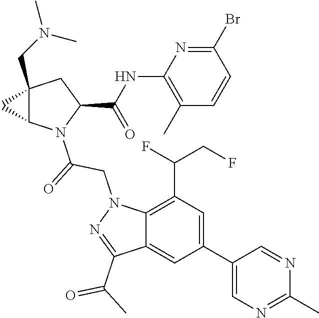 [SMILES: CC(=O)c1nn(CC(=O)N2[C@H](C(=O)Nc3nc(Br)ccc3C)C[C@@]3(CN(C)C)C[C@@H]23)c2c(C(F)CF)cc(-c3cnc(C)nc3)cc12]

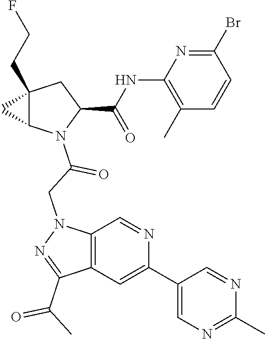 [SMILES: CC(=O)c1nn(CC(=O)N2[C@H](C(=O)Nc3nc(Br)ccc3C)C[C@@]3(CCF)C[C@@H]23)c2cnc(-c3cnc(C)nc3)cc12]

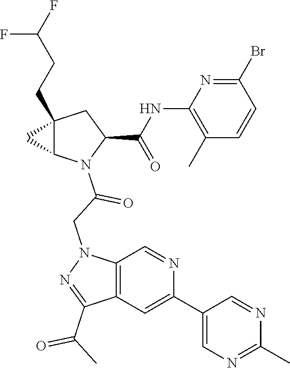 [SMILES: CC(=O)c1nn(CC(=O)N2[C@H](C(=O)Nc3nc(Br)ccc3C)C[C@@]3(CCC(F)F)C[C@@H]23)c2cnc(-c3cnc(C)nc3)cc12]

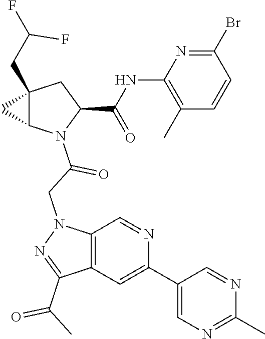 [SMILES: CC(=O)c1nn(CC(=O)N2[C@H](C(=O)Nc3nc(Br)ccc3C)C[C@@]3(CC(F)F)C[C@@H]23)c2cnc(-c3cnc(C)nc3)cc12]

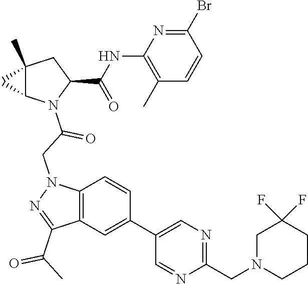 [SMILES: CC(=O)c1nn(CC(=O)N2[C@H](C(=O)Nc3nc(Br)ccc3C)C[C@@]3(C)C[C@@H]23)c2ccc(-c3cnc(CN4CCCC(F)(F)C4)nc3)cc12]

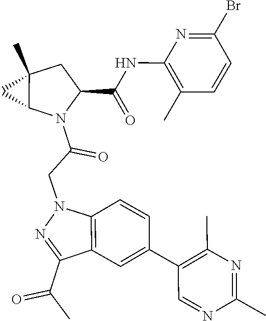 [SMILES: CC(=O)c1nn(CC(=O)N2[C@H](C(=O)Nc3nc(Br)ccc3C)C[C@@]3(C)C[C@@H]23)c2ccc(-c3cnc(C)nc3C)cc12]

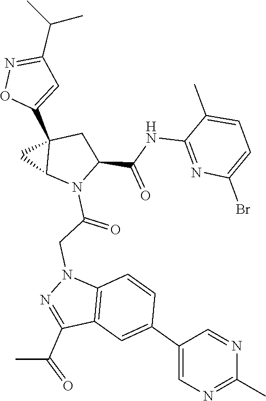 [SMILES: CC(=O)c1nn(CC(=O)N2[C@H](C(=O)Nc3nc(Br)ccc3C)C[C@@]3(c4cc(C(C)C)no4)C[C@@H]23)c2ccc(-c3cnc(C)nc3)cc12]

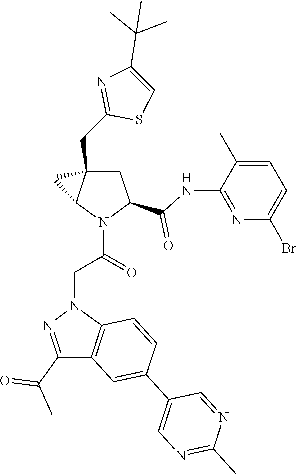

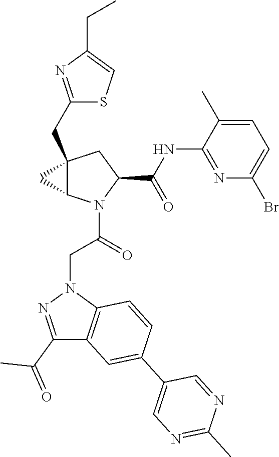 [SMILES: CCc1csc(C[C@@]23C[C@@H](C(=O)Nc4nc(Br)ccc4C)N(C(=O)Cn4nc(C(C)=O)c5cc(-c6cnc(C)nc6)ccc54)[C@@H]2C3)n1]

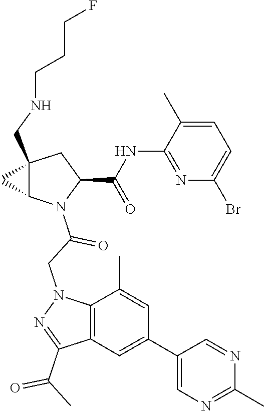 [SMILES: CC(=O)c1nn(CC(=O)N2[C@H](C(=O)Nc3nc(Br)ccc3C)C[C@@]3(CNCCCF)C[C@@H]23)c2c(C)cc(-c3cnc(C)nc3)cc12]

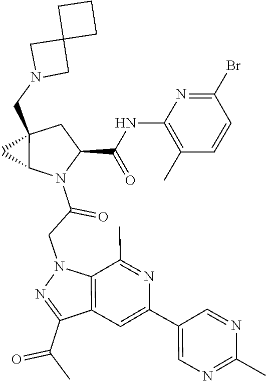 [SMILES: CC(=O)c1nn(CC(=O)N2[C@H](C(=O)Nc3nc(Br)ccc3C)C[C@@]3(CN4CC5(CCC5)C4)C[C@@H]23)c2c(C)nc(-c3cnc(C)nc3)cc12]

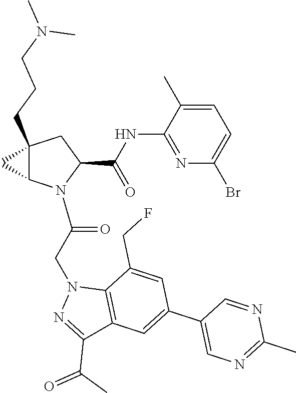 [SMILES: CC(=O)c1nn(CC(=O)N2[C@H](C(=O)Nc3nc(Br)ccc3C)C[C@@]3(CCCN(C)C)C[C@@H]23)c2c(CF)cc(-c3cnc(C)nc3)cc12]

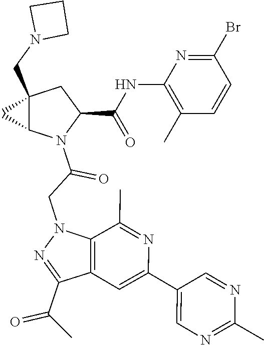 [SMILES: CC(=O)c1nn(CC(=O)N2[C@H](C(=O)Nc3nc(Br)ccc3C)C[C@@]3(CN4CCC4)C[C@@H]23)c2c(C)nc(-c3cnc(C)nc3)cc12]

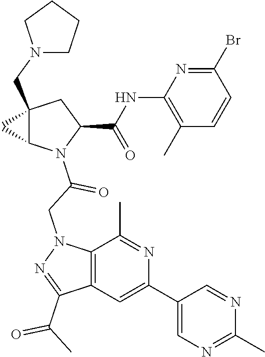 [SMILES: CC(=O)c1nn(CC(=O)N2[C@H](C(=O)Nc3nc(Br)ccc3C)C[C@@]3(CN4CCCC4)C[C@@H]23)c2c(C)nc(-c3cnc(C)nc3)cc12]

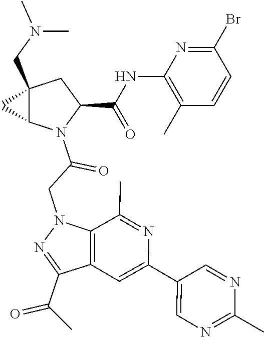 [SMILES: CC(=O)c1nn(CC(=O)N2[C@H](C(=O)Nc3nc(Br)ccc3C)C[C@@]3(CN(C)C)C[C@@H]23)c2c(C)nc(-c3cnc(C)nc3)cc12]

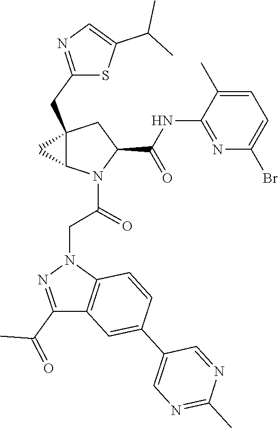 [SMILES: CC(=O)c1nn(CC(=O)N2[C@H](C(=O)Nc3nc(Br)ccc3C)C[C@@]3(Cc4ncc(C(C)C)s4)C[C@@H]23)c2ccc(-c3cnc(C)nc3)cc12]

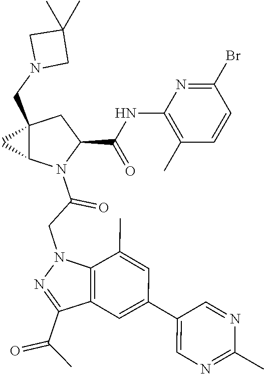 [SMILES: CC(=O)c1nn(CC(=O)N2[C@H](C(=O)Nc3nc(Br)ccc3C)C[C@@]3(CN4CC(C)(C)C4)C[C@@H]23)c2c(C)cc(-c3cnc(C)nc3)cc12]

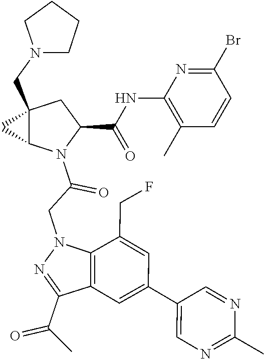 [SMILES: CC(=O)c1nn(CC(=O)N2[C@H](C(=O)Nc3nc(Br)ccc3C)C[C@@]3(CN4CCCC4)C[C@@H]23)c2c(CF)cc(-c3cnc(C)nc3)cc12]

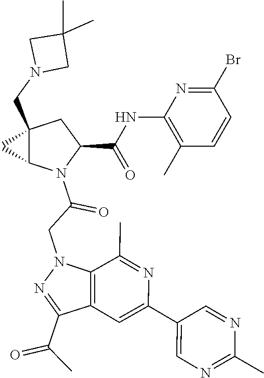 [SMILES: CC(=O)c1nn(CC(=O)N2[C@H](C(=O)Nc3nc(Br)ccc3C)C[C@@]3(CN4CC(C)(C)C4)C[C@@H]23)c2c(C)nc(-c3cnc(C)nc3)cc12]

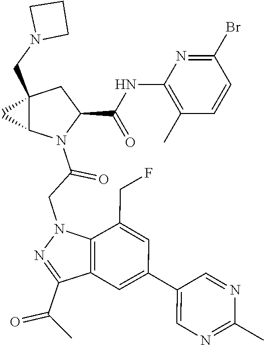 [SMILES: CC(=O)c1nn(CC(=O)N2[C@H](C(=O)Nc3nc(Br)ccc3C)C[C@@]3(CN4CCC4)C[C@@H]23)c2c(CF)cc(-c3cnc(C)nc3)cc12]

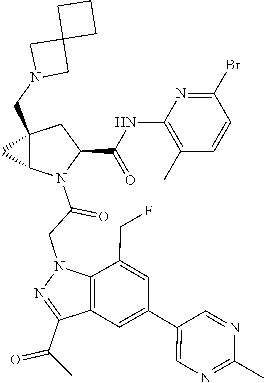 [SMILES: CC(=O)c1nn(CC(=O)N2[C@H](C(=O)Nc3nc(Br)ccc3C)C[C@@]3(CN4CC5(CCC5)C4)C[C@@H]23)c2c(CF)cc(-c3cnc(C)nc3)cc12]

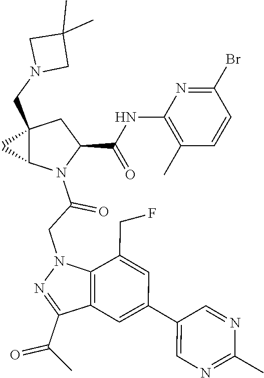 [SMILES: CC(=O)c1nn(CC(=O)N2[C@H](C(=O)Nc3nc(Br)ccc3C)C[C@@]3(CN4CC(C)(C)C4)C[C@@H]23)c2c(CF)cc(-c3cnc(C)nc3)cc12]

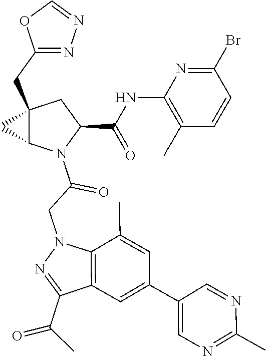 [SMILES: CC(=O)c1nn(CC(=O)N2[C@H](C(=O)Nc3nc(Br)ccc3C)C[C@@]3(Cc4nnco4)C[C@@H]23)c2c(C)cc(-c3cnc(C)nc3)cc12]

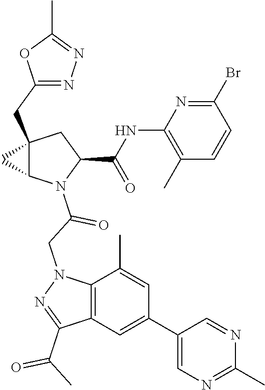 [SMILES: CC(=O)c1nn(CC(=O)N2[C@H](C(=O)Nc3nc(Br)ccc3C)C[C@@]3(Cc4nnc(C)o4)C[C@@H]23)c2c(C)cc(-c3cnc(C)nc3)cc12]

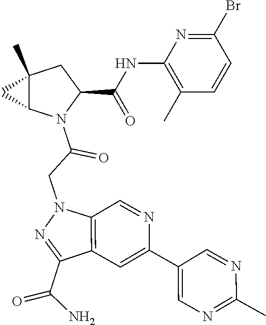 [SMILES: Cc1ncc(-c2cc3c(C(N)=O)nn(CC(=O)N4[C@H](C(=O)Nc5nc(Br)ccc5C)C[C@@]5(C)C[C@@H]45)c3cn2)cn1]

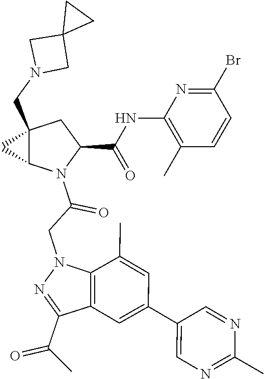 [SMILES: CC(=O)c1nn(CC(=O)N2[C@H](C(=O)Nc3nc(Br)ccc3C)C[C@@]3(CN4CC5(CC5)C4)C[C@@H]23)c2c(C)cc(-c3cnc(C)nc3)cc12]

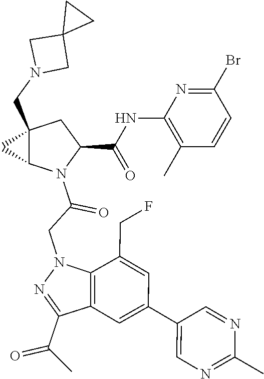 [SMILES: CC(=O)c1nn(CC(=O)N2[C@H](C(=O)Nc3nc(Br)ccc3C)C[C@@]3(CN4CC5(CC5)C4)C[C@@H]23)c2c(CF)cc(-c3cnc(C)nc3)cc12]

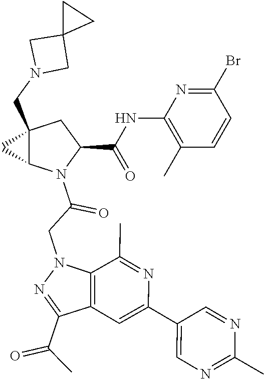 [SMILES: CC(=O)c1nn(CC(=O)N2[C@H](C(=O)Nc3nc(Br)ccc3C)C[C@@]3(CN4CC5(CC5)C4)C[C@@H]23)c2c(C)nc(-c3cnc(C)nc3)cc12]

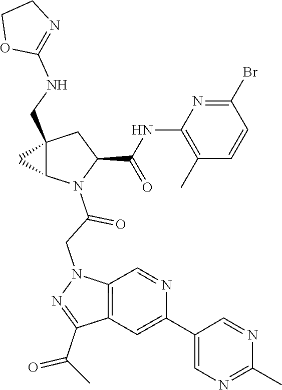 [SMILES: CC(=O)c1nn(CC(=O)N2[C@H](C(=O)Nc3nc(Br)ccc3C)C[C@@]3(CNC4=NCCO4)C[C@@H]23)c2cnc(-c3cnc(C)nc3)cc12]

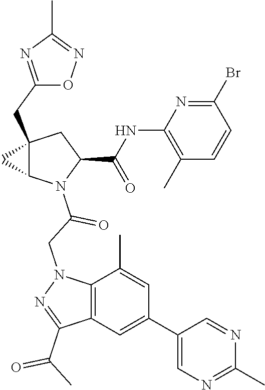 [SMILES: CC(=O)c1nn(CC(=O)N2[C@H](C(=O)Nc3nc(Br)ccc3C)C[C@@]3(Cc4nc(C)no4)C[C@@H]23)c2c(C)cc(-c3cnc(C)nc3)cc12]

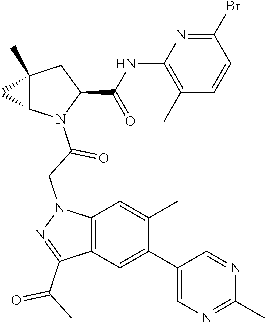 [SMILES: CC(=O)c1nn(CC(=O)N2[C@H](C(=O)Nc3nc(Br)ccc3C)C[C@@]3(C)C[C@@H]23)c2cc(C)c(-c3cnc(C)nc3)cc12]

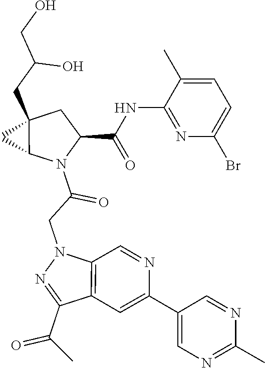 [SMILES: CC(=O)c1nn(CC(=O)N2[C@H](C(=O)Nc3nc(Br)ccc3C)C[C@@]3(CC(O)CO)C[C@@H]23)c2cnc(-c3cnc(C)nc3)cc12]

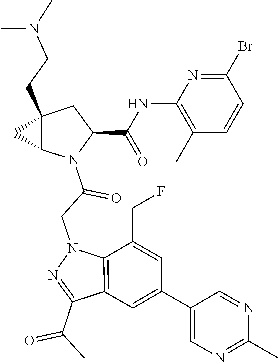

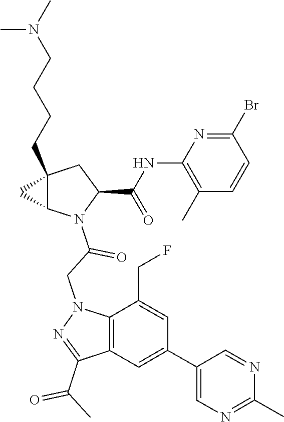

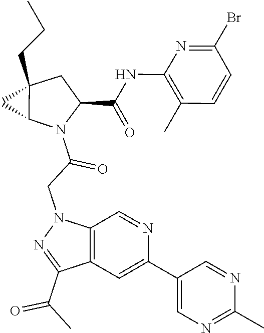 [SMILES: CCC[C@@]12C[C@@H](C(=O)Nc3nc(Br)ccc3C)N(C(=O)Cn3nc(C(C)=O)c4cc(-c5cnc(C)nc5)ncc43)[C@@H]1C2]

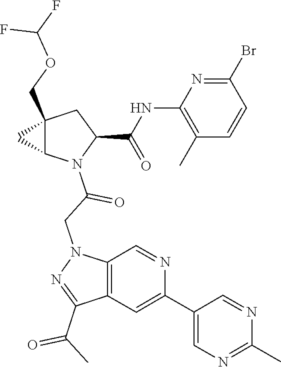 [SMILES: CC(=O)c1nn(CC(=O)N2[C@H](C(=O)Nc3nc(Br)ccc3C)C[C@@]3(COC(F)F)C[C@@H]23)c2cnc(-c3cnc(C)nc3)cc12]

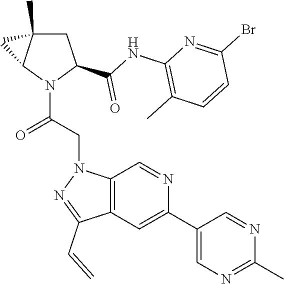 [SMILES: C=Cc1nn(CC(=O)N2[C@H](C(=O)Nc3nc(Br)ccc3C)C[C@@]3(C)C[C@@H]23)c2cnc(-c3cnc(C)nc3)cc12]

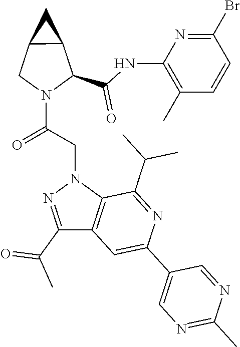 [SMILES: CC(=O)c1nn(CC(=O)N2C[C@H]3C[C@H]3[C@H]2C(=O)Nc2nc(Br)ccc2C)c2c(C(C)C)nc(-c3cnc(C)nc3)cc12]

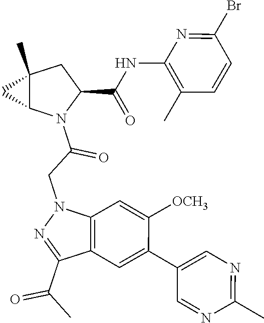 [SMILES: COc1cc2c(cc1-c1cnc(C)nc1)c(C(C)=O)nn2CC(=O)N1[C@H](C(=O)Nc2nc(Br)ccc2C)C[C@@]2(C)C[C@@H]12]

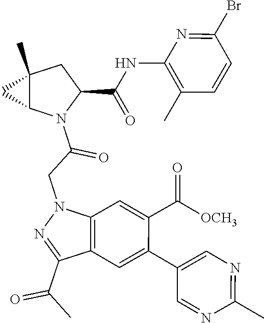 [SMILES: COC(=O)c1cc2c(cc1-c1cnc(C)nc1)c(C(C)=O)nn2CC(=O)N1[C@H](C(=O)Nc2nc(Br)ccc2C)C[C@@]2(C)C[C@@H]12]

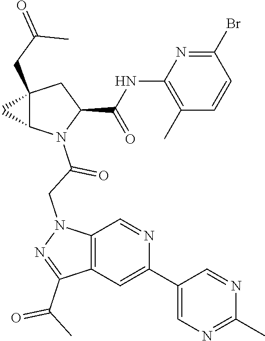 [SMILES: CC(=O)C[C@@]12C[C@@H](C(=O)Nc3nc(Br)ccc3C)N(C(=O)Cn3nc(C(C)=O)c4cc(-c5cnc(C)nc5)ncc43)[C@@H]1C2]

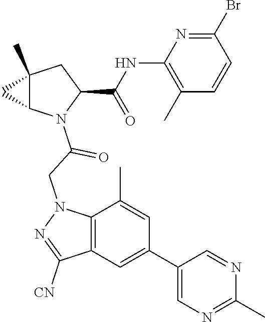

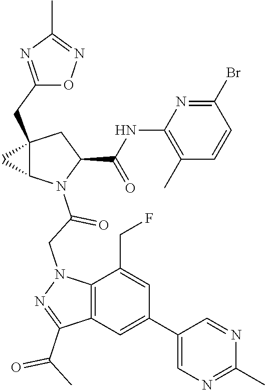 [SMILES: CC(=O)c1nn(CC(=O)N2[C@H](C(=O)Nc3nc(Br)ccc3C)C[C@@]3(Cc4nc(C)no4)C[C@@H]23)c2c(CF)cc(-c3cnc(C)nc3)cc12]

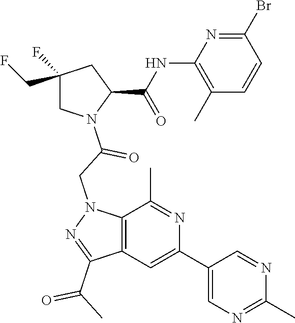 [SMILES: CC(=O)c1nn(CC(=O)N2C[C@@](F)(CF)C[C@H]2C(=O)Nc2nc(Br)ccc2C)c2c(C)nc(-c3cnc(C)nc3)cc12]

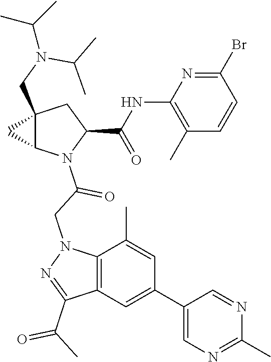 [SMILES: CC(=O)c1nn(CC(=O)N2[C@H](C(=O)Nc3nc(Br)ccc3C)C[C@@]3(CN(C(C)C)C(C)C)C[C@@H]23)c2c(C)cc(-c3cnc(C)nc3)cc12]

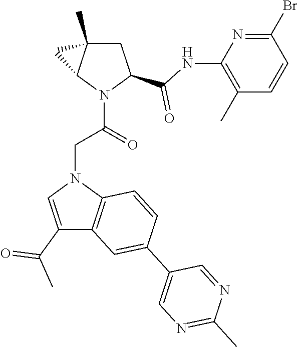 [SMILES: CC(=O)c1cn(CC(=O)N2[C@H](C(=O)Nc3nc(Br)ccc3C)C[C@@]3(C)C[C@@H]23)c2ccc(-c3cnc(C)nc3)cc12]

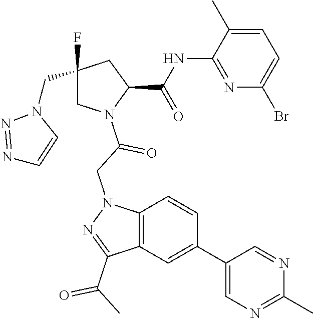 [SMILES: CC(=O)c1nn(CC(=O)N2C[C@](F)(Cn3ccnn3)C[C@H]2C(=O)Nc2nc(Br)ccc2C)c2ccc(-c3cnc(C)nc3)cc12]

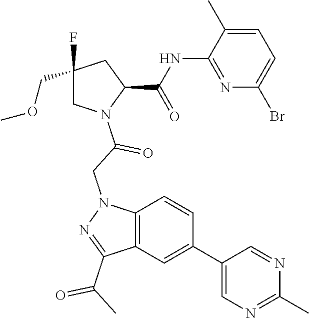

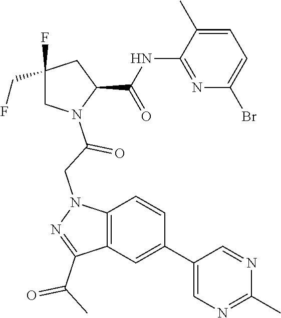 [SMILES: CC(=O)c1nn(CC(=O)N2C[C@](F)(CF)C[C@H]2C(=O)Nc2nc(Br)ccc2C)c2ccc(-c3cnc(C)nc3)cc12]

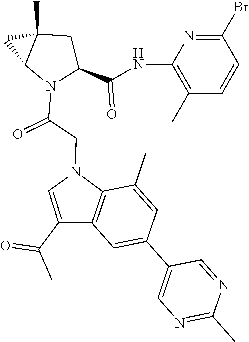 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]

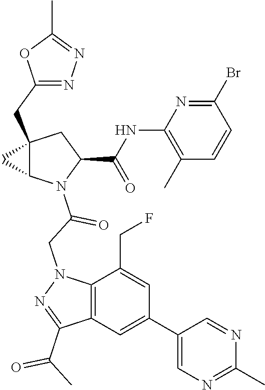 [SMILES: CC(=O)c1nn(CC(=O)N2[C@H](C(=O)Nc3nc(Br)ccc3C)C[C@@]3(Cc4nnc(C)o4)C[C@@H]23)c2c(CF)cc(-c3cnc(C)nc3)cc12]

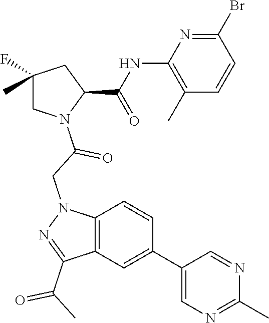

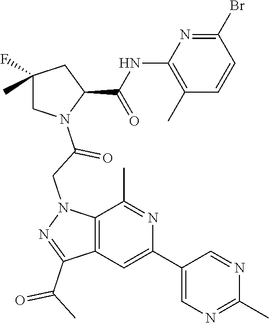

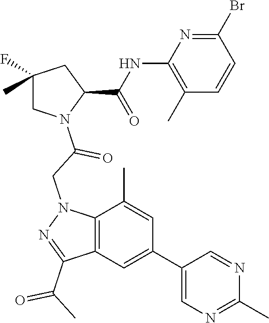

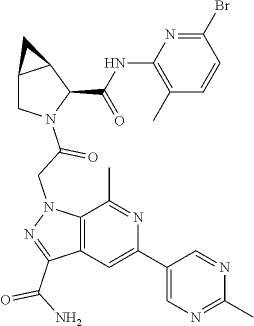 [SMILES: Cc1ncc(-c2cc3c(C(N)=O)nn(CC(=O)N4C[C@H]5C[C@H]5[C@H]4C(=O)Nc4nc(Br)ccc4C)c3c(C)n2)cn1]

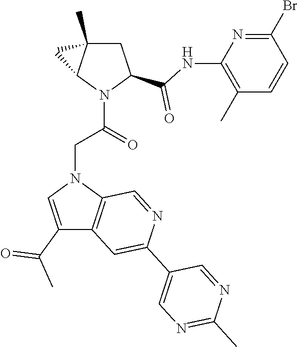 [SMILES: CC(=O)c1cn(CC(=O)N2[C@H](C(=O)Nc3nc(Br)ccc3C)C[C@@]3(C)C[C@@H]23)c2cnc(-c3cnc(C)nc3)cc12]

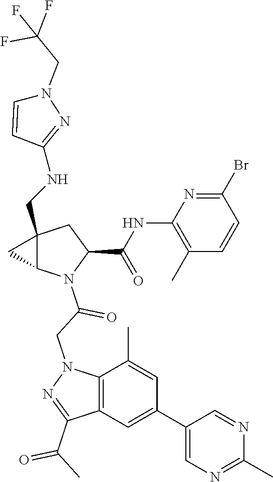 [SMILES: CC(=O)c1nn(CC(=O)N2[C@H](C(=O)Nc3nc(Br)ccc3C)C[C@@]3(CNc4ccn(CC(F)(F)F)n4)C[C@@H]23)c2c(C)cc(-c3cnc(C)nc3)cc12]

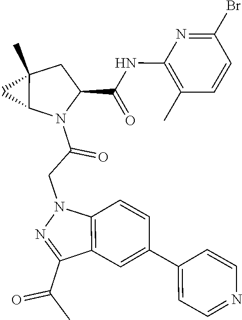 [SMILES: CC(=O)c1nn(CC(=O)N2[C@H](C(=O)Nc3nc(Br)ccc3C)C[C@@]3(C)C[C@@H]23)c2ccc(-c3ccncc3)cc12]

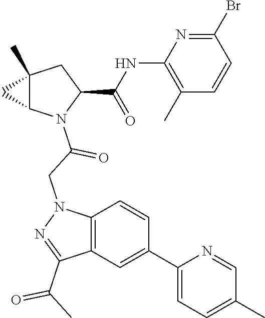 [SMILES: CC(=O)c1nn(CC(=O)N2[C@H](C(=O)Nc3nc(Br)ccc3C)C[C@@]3(C)C[C@@H]23)c2ccc(-c3ccc(C)cn3)cc12]

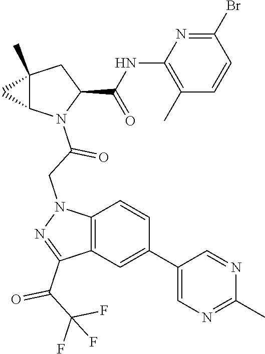 [SMILES: Cc1ncc(-c2ccc3c(c2)c(C(=O)C(F)(F)F)nn3CC(=O)N2[C@H](C(=O)Nc3nc(Br)ccc3C)C[C@@]3(C)C[C@@H]23)cn1]

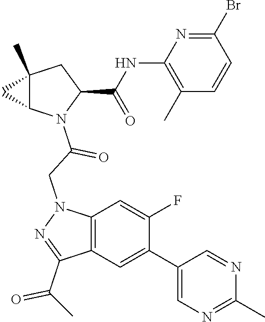 [SMILES: CC(=O)c1nn(CC(=O)N2[C@H](C(=O)Nc3nc(Br)ccc3C)C[C@@]3(C)C[C@@H]23)c2cc(F)c(-c3cnc(C)nc3)cc12]

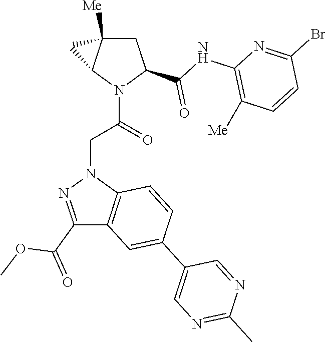 [SMILES: COC(=O)c1nn(CC(=O)N2[C@H](C(=O)Nc3nc(Br)ccc3C)C[C@@]3(C)C[C@@H]23)c2ccc(-c3cnc(C)nc3)cc12]

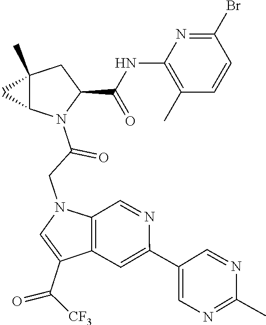 [SMILES: Cc1ncc(-c2cc3c(C(=O)C(F)(F)F)cn(CC(=O)N4[C@H](C(=O)Nc5nc(Br)ccc5C)C[C@@]5(C)C[C@@H]45)c3cn2)cn1]

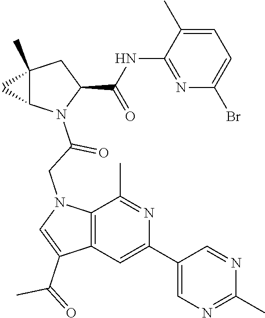 [SMILES: CC(=O)c1cn(CC(=O)N2[C@H](C(=O)Nc3nc(Br)ccc3C)C[C@@]3(C)C[C@@H]23)c2c(C)nc(-c3cnc(C)nc3)cc12]

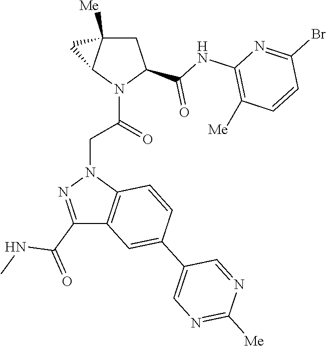 [SMILES: CNC(=O)c1nn(CC(=O)N2[C@H](C(=O)Nc3nc(Br)ccc3C)C[C@@]3(C)C[C@@H]23)c2ccc(-c3cnc(C)nc3)cc12]

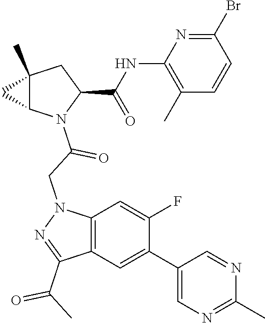 [SMILES: CC(=O)c1nn(CC(=O)N2[C@H](C(=O)Nc3nc(Br)ccc3C)C[C@@]3(C)C[C@@H]23)c2cc(F)c(-c3cnc(C)nc3)cc12]

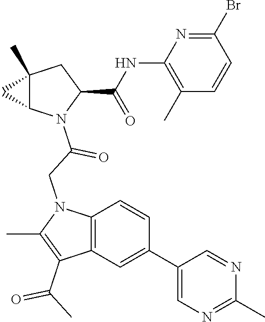 [SMILES: CC(=O)c1c(C)n(CC(=O)N2[C@H](C(=O)Nc3nc(Br)ccc3C)C[C@@]3(C)C[C@@H]23)c2ccc(-c3cnc(C)nc3)cc12]

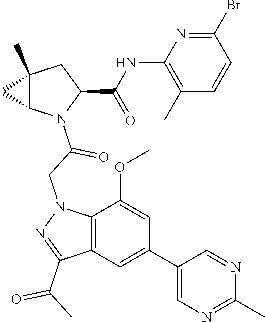 [SMILES: COc1cc(-c2cnc(C)nc2)cc2c(C(C)=O)nn(CC(=O)N3[C@H](C(=O)Nc4nc(Br)ccc4C)C[C@@]4(C)C[C@@H]34)c12]

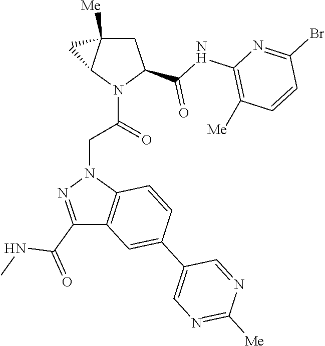 [SMILES: CNC(=O)c1nn(CC(=O)N2[C@H](C(=O)Nc3nc(Br)ccc3C)C[C@@]3(C)C[C@@H]23)c2ccc(-c3cnc(C)nc3)cc12]

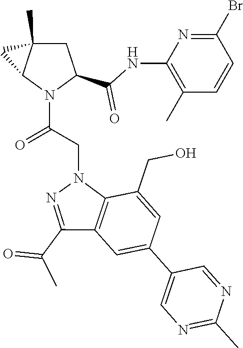 [SMILES: CC(=O)c1nn(CC(=O)N2[C@H](C(=O)Nc3nc(Br)ccc3C)C[C@@]3(C)C[C@@H]23)c2c(CO)cc(-c3cnc(C)nc3)cc12]

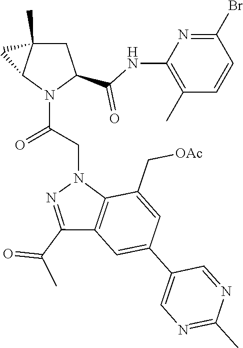 [SMILES: CC(=O)OCc1cc(-c2cnc(C)nc2)cc2c(C(C)=O)nn(CC(=O)N3[C@H](C(=O)Nc4nc(Br)ccc4C)C[C@@]4(C)C[C@@H]34)c12]

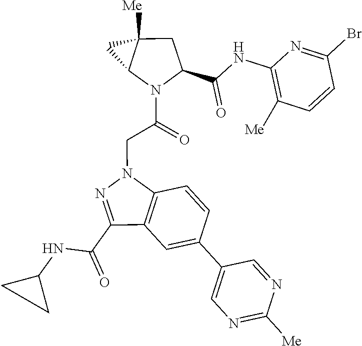 [SMILES: Cc1ncc(-c2ccc3c(c2)c(C(=O)NC2CC2)nn3CC(=O)N2[C@H](C(=O)Nc3nc(Br)ccc3C)C[C@@]3(C)C[C@@H]23)cn1]

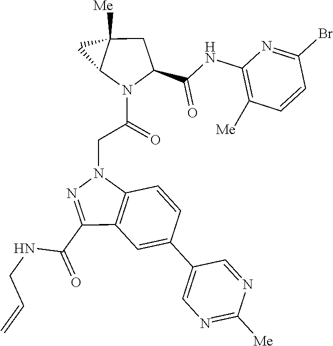 [SMILES: C=CCNC(=O)c1nn(CC(=O)N2[C@H](C(=O)Nc3nc(Br)ccc3C)C[C@@]3(C)C[C@@H]23)c2ccc(-c3cnc(C)nc3)cc12]

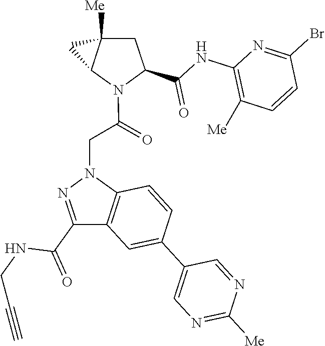 [SMILES: C#CCNC(=O)c1nn(CC(=O)N2[C@H](C(=O)Nc3nc(Br)ccc3C)C[C@@]3(C)C[C@@H]23)c2ccc(-c3cnc(C)nc3)cc12]

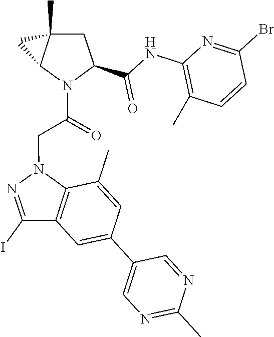 [SMILES: Cc1ncc(-c2cc(C)c3c(c2)c(I)nn3CC(=O)N2[C@H](C(=O)Nc3nc(Br)ccc3C)C[C@@]3(C)C[C@@H]23)cn1]

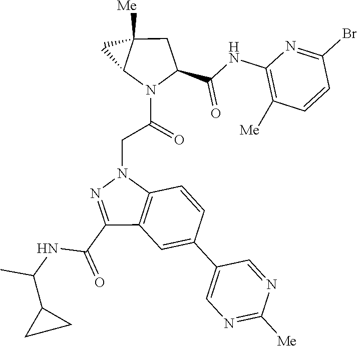 [SMILES: Cc1ncc(-c2ccc3c(c2)c(C(=O)NC(C)C2CC2)nn3CC(=O)N2[C@H](C(=O)Nc3nc(Br)ccc3C)C[C@@]3(C)C[C@@H]23)cn1]

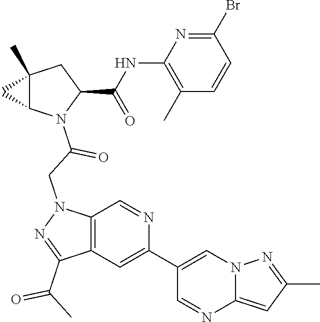 [SMILES: CC(=O)c1nn(CC(=O)N2[C@H](C(=O)Nc3nc(Br)ccc3C)C[C@@]3(C)C[C@@H]23)c2cnc(-c3cnc4cc(C)nn4c3)cc12]

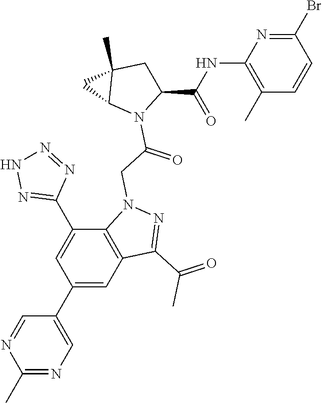 [SMILES: CC(=O)c1nn(CC(=O)N2[C@H](C(=O)Nc3nc(Br)ccc3C)C[C@@]3(C)C[C@@H]23)c2c(-c3nn[nH]n3)cc(-c3cnc(C)nc3)cc12]

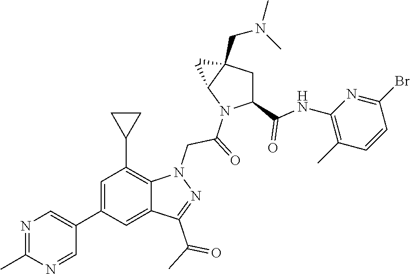 [SMILES: CC(=O)c1nn(CC(=O)N2[C@H](C(=O)Nc3nc(Br)ccc3C)C[C@@]3(CN(C)C)C[C@@H]23)c2c(C3CC3)cc(-c3cnc(C)nc3)cc12]

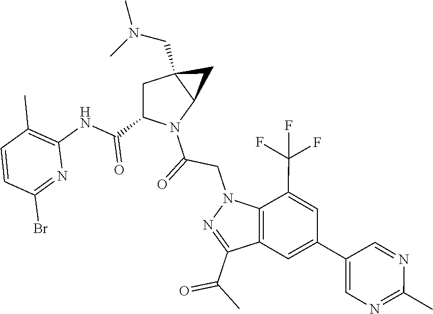 [SMILES: CC(=O)c1nn(CC(=O)N2[C@H](C(=O)Nc3nc(Br)ccc3C)C[C@@]3(CN(C)C)C[C@@H]23)c2c(C(F)(F)F)cc(-c3cnc(C)nc3)cc12]

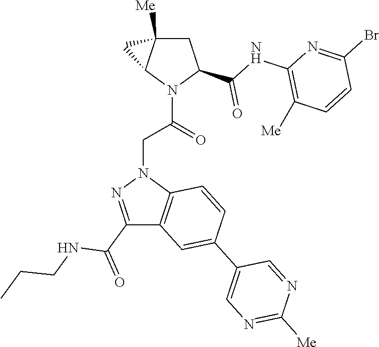 [SMILES: CCCNC(=O)c1nn(CC(=O)N2[C@H](C(=O)Nc3nc(Br)ccc3C)C[C@@]3(C)C[C@@H]23)c2ccc(-c3cnc(C)nc3)cc12]

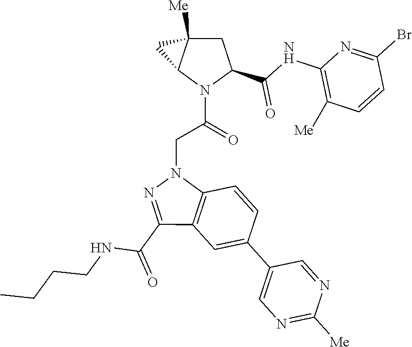 [SMILES: CCCCNC(=O)c1nn(CC(=O)N2[C@H](C(=O)Nc3nc(Br)ccc3C)C[C@@]3(C)C[C@@H]23)c2ccc(-c3cnc(C)nc3)cc12]

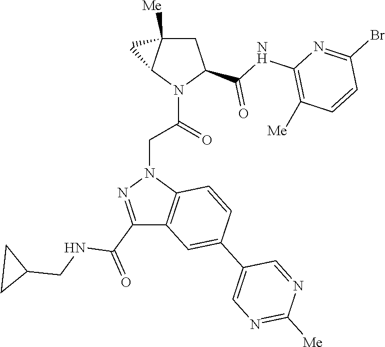 [SMILES: Cc1ncc(-c2ccc3c(c2)c(C(=O)NCC2CC2)nn3CC(=O)N2[C@H](C(=O)Nc3nc(Br)ccc3C)C[C@@]3(C)C[C@@H]23)cn1]

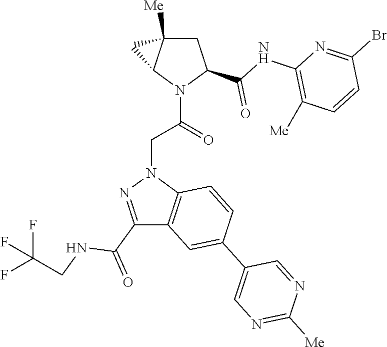 [SMILES: Cc1ncc(-c2ccc3c(c2)c(C(=O)NCC(F)(F)F)nn3CC(=O)N2[C@H](C(=O)Nc3nc(Br)ccc3C)C[C@@]3(C)C[C@@H]23)cn1]

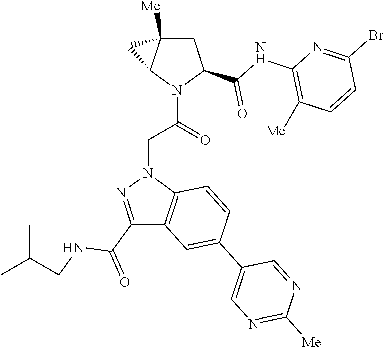 [SMILES: Cc1ncc(-c2ccc3c(c2)c(C(=O)NCC(C)C)nn3CC(=O)N2[C@H](C(=O)Nc3nc(Br)ccc3C)C[C@@]3(C)C[C@@H]23)cn1]

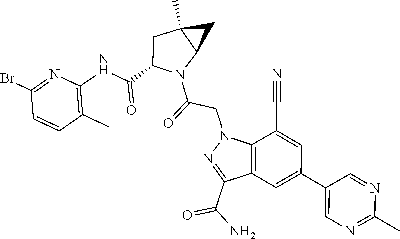 [SMILES: Cc1ncc(-c2cc(C#N)c3c(c2)c(C(N)=O)nn3CC(=O)N2[C@H](C(=O)Nc3nc(Br)ccc3C)C[C@@]3(C)C[C@@H]23)cn1]

In some embodiments, the compound of formula (V) is:

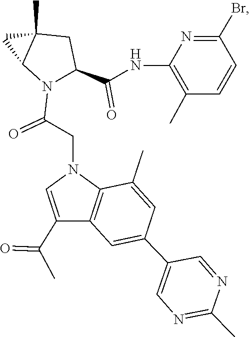 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]

or a pharmaceutically acceptable salt thereof.

In some embodiments, the compound of formula (V) is a compound disclosed in WO2020/041301A1. For example, the compound of formula (V) is a compound of Table 3 or a pharmaceutically acceptable salt thereof.

TABLE 3

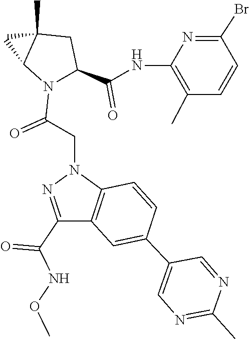 [SMILES: CONC(=O)c1nn(CC(=O)N2[C@H](C(=O)Nc3nc(Br)ccc3C)C[C@@]3(C)C[C@@H]23)c2ccc(-c3cnc(C)nc3)cc12]

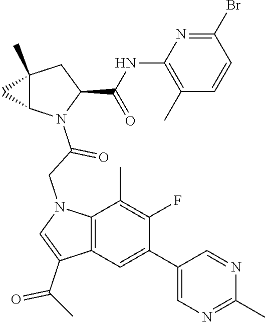 [SMILES: CC(=O)c1cn(CC(=O)N2[C@H](C(=O)Nc3nc(Br)ccc3C)C[C@@]3(C)C[C@@H]23)c2c(C)c(F)c(-c3cnc(C)nc3)cc12]

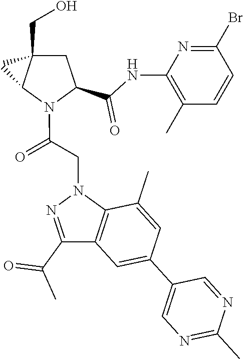 [SMILES: CC(=O)c1nn(CC(=O)N2[C@H](C(=O)Nc3nc(Br)ccc3C)C[C@@]3(CO)C[C@@H]23)c2c(C)cc(-c3cnc(C)nc3)cc12]

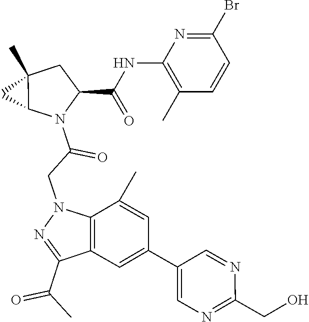 [SMILES: CC(=O)c1nn(CC(=O)N2[C@H](C(=O)Nc3nc(Br)ccc3C)C[C@@]3(C)C[C@@H]23)c2c(C)cc(-c3cnc(CO)nc3)cc12]

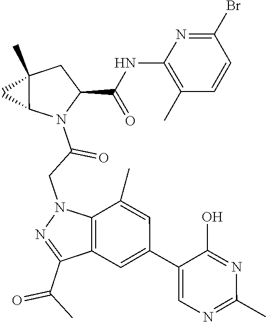 [SMILES: CC(=O)c1nn(CC(=O)N2[C@H](C(=O)Nc3nc(Br)ccc3C)C[C@@]3(C)C[C@@H]23)c2c(C)cc(-c3cnc(C)nc3O)cc12]

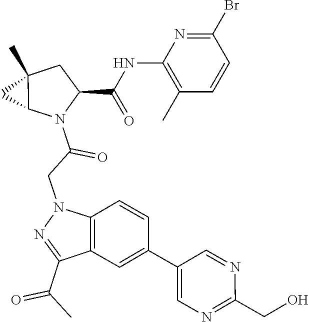 [SMILES: CC(=O)c1nn(CC(=O)N2[C@H](C(=O)Nc3nc(Br)ccc3C)C[C@@]3(C)C[C@@H]23)c2ccc(-c3cnc(CO)nc3)cc12]

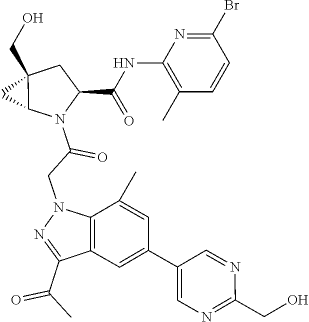 [SMILES: CC(=O)c1nn(CC(=O)N2[C@H](C(=O)Nc3nc(Br)ccc3C)C[C@@]3(CO)C[C@@H]23)c2c(C)cc(-c3cnc(CO)nc3)cc12]

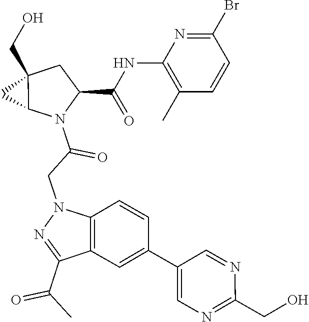 [SMILES: CC(=O)c1nn(CC(=O)N2[C@H](C(=O)Nc3nc(Br)ccc3C)C[C@@]3(CO)C[C@@H]23)c2ccc(-c3cnc(CO)nc3)cc12]

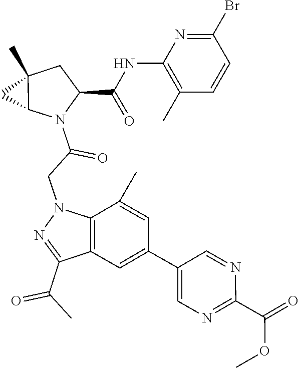 [SMILES: COC(=O)c1ncc(-c2cc(C)c3c(c2)c(C(C)=O)nn3CC(=O)N2[C@H](C(=O)Nc3nc(Br)ccc3C)C[C@@]3(C)C[C@@H]23)cn1]

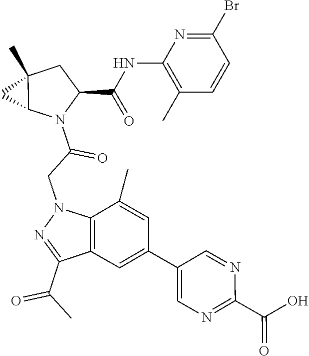 [SMILES: CC(=O)c1nn(CC(=O)N2[C@H](C(=O)Nc3nc(Br)ccc3C)C[C@@]3(C)C[C@@H]23)c2c(C)cc(-c3cnc(C(=O)O)nc3)cc12]

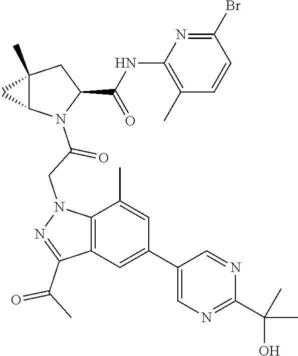 [SMILES: CC(=O)c1nn(CC(=O)N2[C@H](C(=O)Nc3nc(Br)ccc3C)C[C@@]3(C)C[C@@H]23)c2c(C)cc(-c3cnc(C(C)(C)O)nc3)cc12]

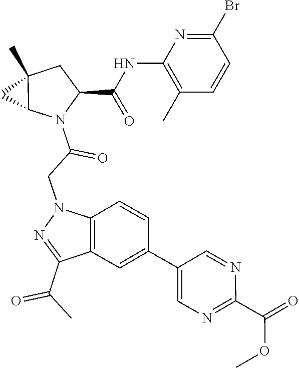 [SMILES: COC(=O)c1ncc(-c2ccc3c(c2)c(C(C)=O)nn3CC(=O)N2[C@H](C(=O)Nc3nc(Br)ccc3C)C[C@@]3(C)C[C@@H]23)cn1]

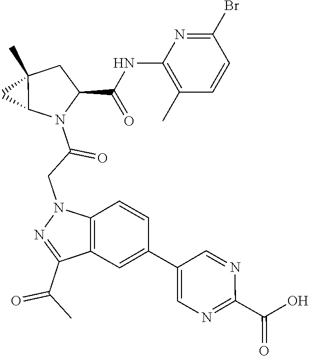 [SMILES: CC(=O)c1nn(CC(=O)N2[C@H](C(=O)Nc3nc(Br)ccc3C)C[C@@]3(C)C[C@@H]23)c2ccc(-c3cnc(C(=O)O)nc3)cc12]

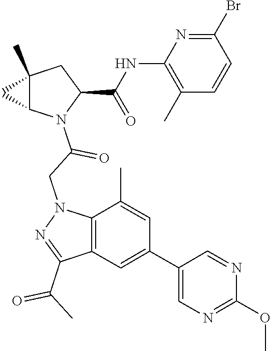 [SMILES: COc1ncc(-c2cc(C)c3c(c2)c(C(C)=O)nn3CC(=O)N2[C@H](C(=O)Nc3nc(Br)ccc3C)C[C@@]3(C)C[C@@H]23)cn1]

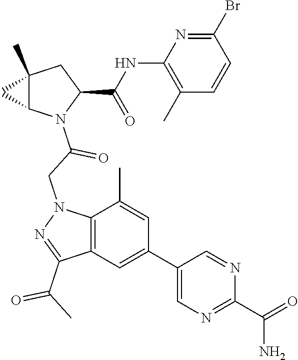 [SMILES: CC(=O)c1nn(CC(=O)N2[C@H](C(=O)Nc3nc(Br)ccc3C)C[C@@]3(C)C[C@@H]23)c2c(C)cc(-c3cnc(C(N)=O)nc3)cc12]

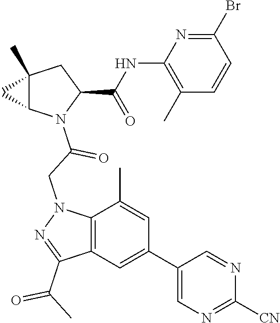 [SMILES: CC(=O)c1nn(CC(=O)N2[C@H](C(=O)Nc3nc(Br)ccc3C)C[C@@]3(C)C[C@@H]23)c2c(C)cc(-c3cnc(C#N)nc3)cc12]

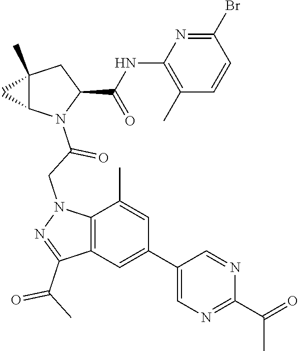 [SMILES: CC(=O)c1ncc(-c2cc(C)c3c(c2)c(C(C)=O)nn3CC(=O)N2[C@H](C(=O)Nc3nc(Br)ccc3C)C[C@@]3(C)C[C@@H]23)cn1]

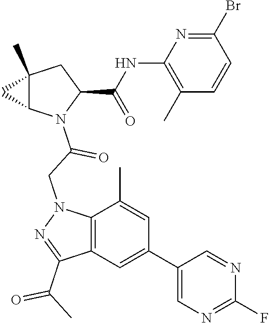 [SMILES: CC(=O)c1nn(CC(=O)N2[C@H](C(=O)Nc3nc(Br)ccc3C)C[C@@]3(C)C[C@@H]23)c2c(C)cc(-c3cnc(F)nc3)cc12]

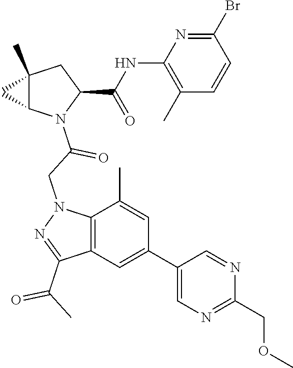 [SMILES: COCc1ncc(-c2cc(C)c3c(c2)c(C(C)=O)nn3CC(=O)N2[C@H](C(=O)Nc3nc(Br)ccc3C)C[C@@]3(C)C[C@@H]23)cn1]

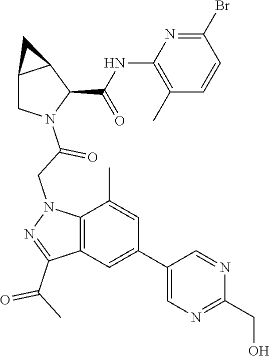 [SMILES: CC(=O)c1nn(CC(=O)N2C[C@H]3C[C@H]3[C@H]2C(=O)Nc2nc(Br)ccc2C)c2c(C)cc(-c3cnc(CO)nc3)cc12]

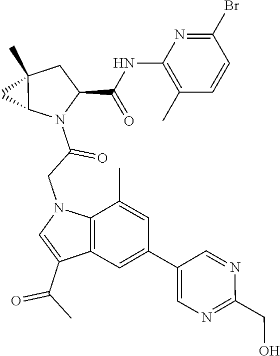 [SMILES: CC(=O)c1cn(CC(=O)N2[C@H](C(=O)Nc3nc(Br)ccc3C)C[C@@]3(C)C[C@@H]23)c2c(C)cc(-c3cnc(CO)nc3)cc12]

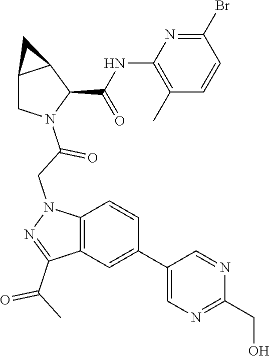 [SMILES: CC(=O)c1nn(CC(=O)N2C[C@H]3C[C@H]3[C@H]2C(=O)Nc2nc(Br)ccc2C)c2ccc(-c3cnc(CO)nc3)cc12]

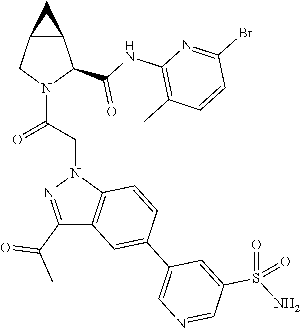 [SMILES: CC(=O)c1nn(CC(=O)N2C[C@H]3C[C@H]3[C@H]2C(=O)Nc2nc(Br)ccc2C)c2ccc(-c3cncc(S(N)(=O)=O)c3)cc12]

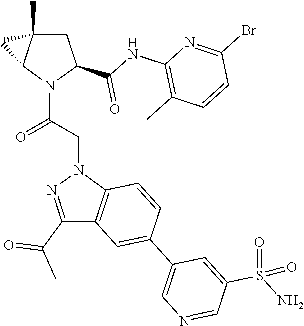 [SMILES: CC(=O)c1nn(CC(=O)N2[C@H](C(=O)Nc3nc(Br)ccc3C)C[C@@]3(C)C[C@@H]23)c2ccc(-c3cncc(S(N)(=O)=O)c3)cc12]

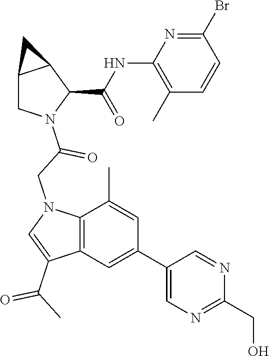 [SMILES: CC(=O)c1cn(CC(=O)N2C[C@H]3C[C@H]3[C@H]2C(=O)Nc2nc(Br)ccc2C)c2c(C)cc(-c3cnc(CO)nc3)cc12]

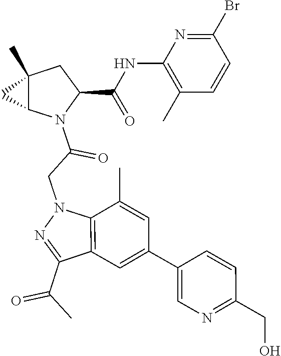 [SMILES: CC(=O)c1nn(CC(=O)N2[C@H](C(=O)Nc3nc(Br)ccc3C)C[C@@]3(C)C[C@@H]23)c2c(C)cc(-c3ccc(CO)nc3)cc12]

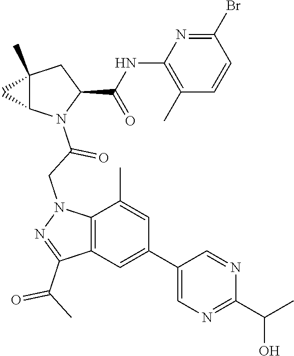 [SMILES: CC(=O)c1nn(CC(=O)N2[C@H](C(=O)Nc3nc(Br)ccc3C)C[C@@]3(C)C[C@@H]23)c2c(C)cc(-c3cnc(C(C)O)nc3)cc12]

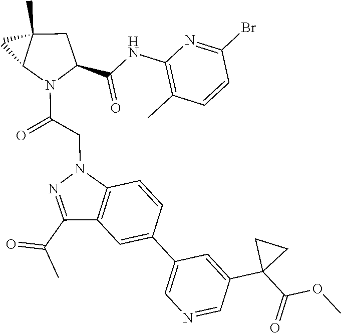 [SMILES: COC(=O)C1(c2cncc(-c3ccc4c(c3)c(C(C)=O)nn4CC(=O)N3[C@H](C(=O)Nc4nc(Br)ccc4C)C[C@@]4(C)C[C@@H]34)c2)CC1]

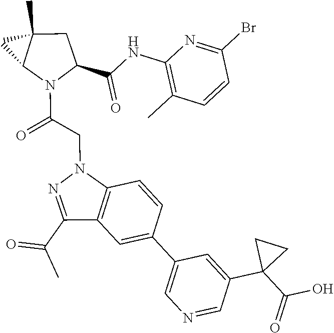 [SMILES: CC(=O)c1nn(CC(=O)N2[C@H](C(=O)Nc3nc(Br)ccc3C)C[C@@]3(C)C[C@@H]23)c2ccc(-c3cncc(C4(C(=O)O)CC4)c3)cc12]

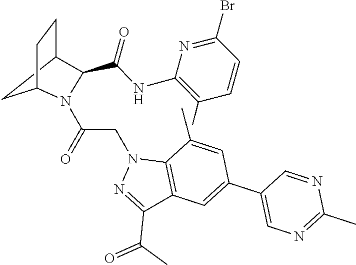 [SMILES: CC(=O)c1nn(CC(=O)N2C3CCC(C3)[C@H]2C(=O)Nc2nc(Br)ccc2C)c2c(C)cc(-c3cnc(C)nc3)cc12]

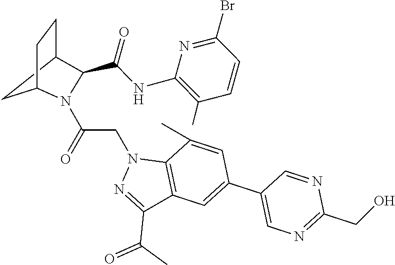 [SMILES: CC(=O)c1nn(CC(=O)N2C3CCC(C3)[C@H]2C(=O)Nc2nc(Br)ccc2C)c2c(C)cc(-c3cnc(CO)nc3)cc12]

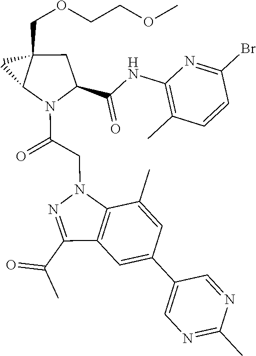 [SMILES: COCCOC[C@@]12C[C@@H](C(=O)Nc3nc(Br)ccc3C)N(C(=O)Cn3nc(C(C)=O)c4cc(-c5cnc(C)nc5)cc(C)c43)[C@@H]1C2]

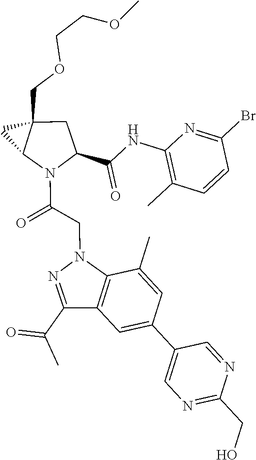 [SMILES: COCCOC[C@@]12C[C@@H](C(=O)Nc3nc(Br)ccc3C)N(C(=O)Cn3nc(C(C)=O)c4cc(-c5cnc(CO)nc5)cc(C)c43)[C@@H]1C2]

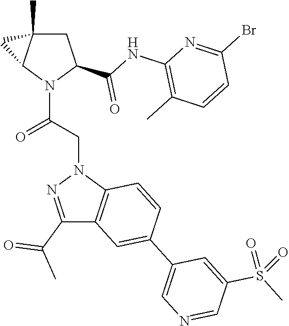 [SMILES: CC(=O)c1nn(CC(=O)N2[C@H](C(=O)Nc3nc(Br)ccc3C)C[C@@]3(C)C[C@@H]23)c2ccc(-c3cncc(S(C)(=O)=O)c3)cc12]

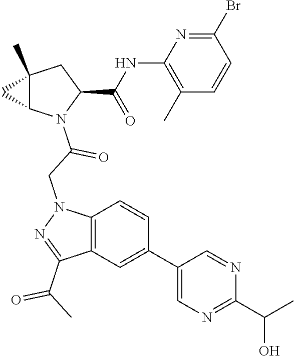 [SMILES: CC(=O)c1nn(CC(=O)N2[C@H](C(=O)Nc3nc(Br)ccc3C)C[C@@]3(C)C[C@@H]23)c2ccc(-c3cnc(C(C)O)nc3)cc12]

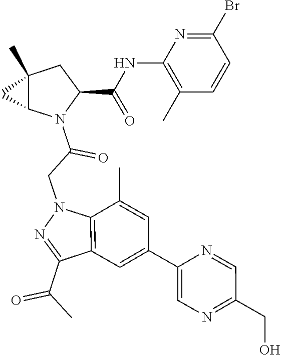 [SMILES: CC(=O)c1nn(CC(=O)N2[C@H](C(=O)Nc3nc(Br)ccc3C)C[C@@]3(C)C[C@@H]23)c2c(C)cc(-c3cnc(CO)cn3)cc12]

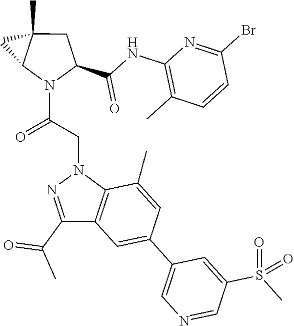 [SMILES: CC(=O)c1nn(CC(=O)N2[C@H](C(=O)Nc3nc(Br)ccc3C)C[C@@]3(C)C[C@@H]23)c2c(C)cc(-c3cncc(S(C)(=O)=O)c3)cc12]

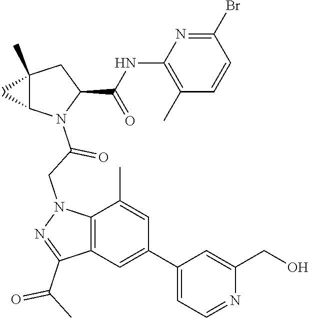 [SMILES: CC(=O)c1nn(CC(=O)N2[C@H](C(=O)Nc3nc(Br)ccc3C)C[C@@]3(C)C[C@@H]23)c2c(C)cc(-c3ccnc(CO)c3)cc12]

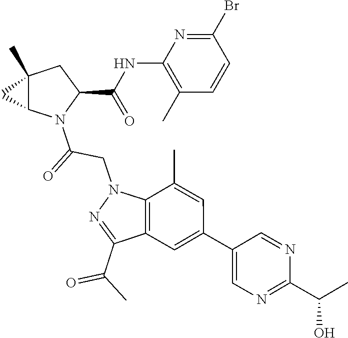 [SMILES: CC(=O)c1nn(CC(=O)N2[C@H](C(=O)Nc3nc(Br)ccc3C)C[C@@]3(C)C[C@@H]23)c2c(C)cc(-c3cnc([C@H](C)O)nc3)cc12]

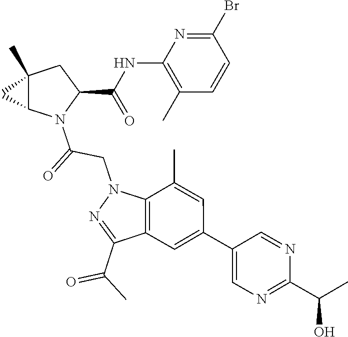 [SMILES: CC(=O)c1nn(CC(=O)N2[C@H](C(=O)Nc3nc(Br)ccc3C)C[C@@]3(C)C[C@@H]23)c2c(C)cc(-c3cnc([C@@H](C)O)nc3)cc12]

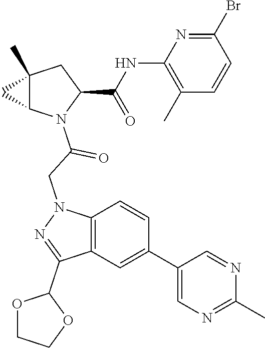 [SMILES: Cc1ncc(-c2ccc3c(c2)c(C2OCCO2)nn3CC(=O)N2[C@H](C(=O)Nc3nc(Br)ccc3C)C[C@@]3(C)C[C@@H]23)cn1]

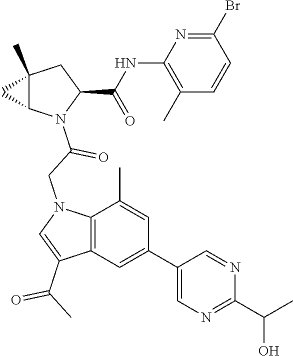 [SMILES: CC(=O)c1cn(CC(=O)N2[C@H](C(=O)Nc3nc(Br)ccc3C)C[C@@]3(C)C[C@@H]23)c2c(C)cc(-c3cnc(C(C)O)nc3)cc12]

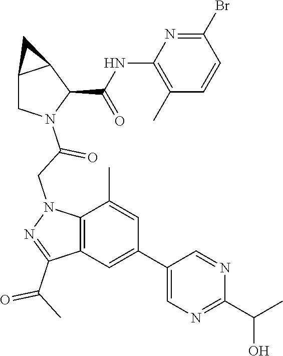 [SMILES: CC(=O)c1nn(CC(=O)N2C[C@H]3C[C@H]3[C@H]2C(=O)Nc2nc(Br)ccc2C)c2c(C)cc(-c3cnc(C(C)O)nc3)cc12]

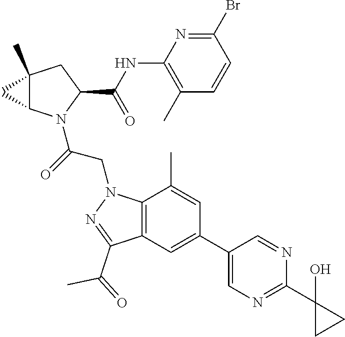 [SMILES: CC(=O)c1nn(CC(=O)N2[C@H](C(=O)Nc3nc(Br)ccc3C)C[C@@]3(C)C[C@@H]23)c2c(C)cc(-c3cnc(C4(O)CC4)nc3)cc12]

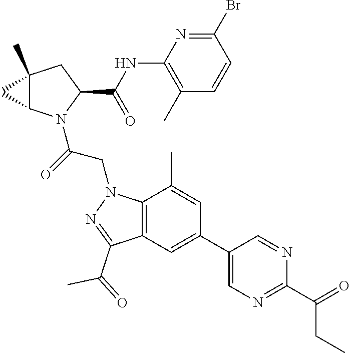 [SMILES: CCC(=O)c1ncc(-c2cc(C)c3c(c2)c(C(C)=O)nn3CC(=O)N2[C@H](C(=O)Nc3nc(Br)ccc3C)C[C@@]3(C)C[C@@H]23)cn1]

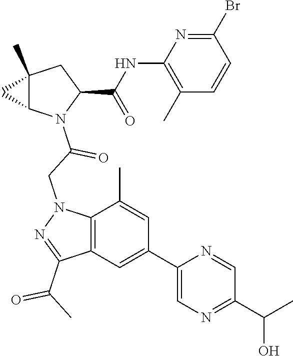 [SMILES: CC(=O)c1nn(CC(=O)N2[C@H](C(=O)Nc3nc(Br)ccc3C)C[C@@]3(C)C[C@@H]23)c2c(C)cc(-c3cnc(C(C)O)cn3)cc12]

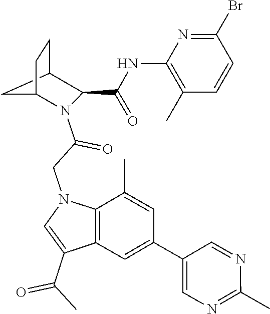 [SMILES: CC(=O)c1cn(CC(=O)N2C3CCC(C3)[C@H]2C(=O)Nc2nc(Br)ccc2C)c2c(C)cc(-c3cnc(C)nc3)cc12]

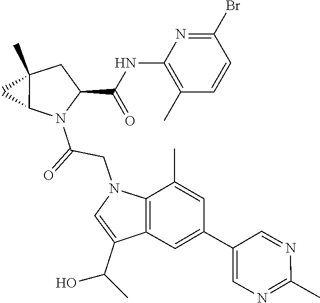 [SMILES: Cc1ncc(-c2cc(C)c3c(c2)c(C(C)O)cn3CC(=O)N2[C@H](C(=O)Nc3nc(Br)ccc3C)C[C@@]3(C)C[C@@H]23)cn1]

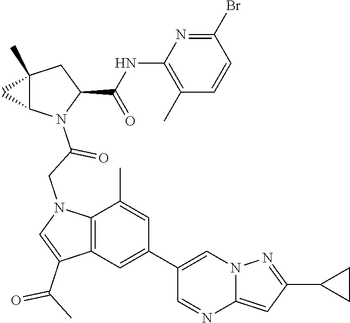 [SMILES: CC(=O)c1cn(CC(=O)N2[C@H](C(=O)Nc3nc(Br)ccc3C)C[C@@]3(C)C[C@@H]23)c2c(C)cc(-c3cnc4cc(C5CC5)nn4c3)cc12]

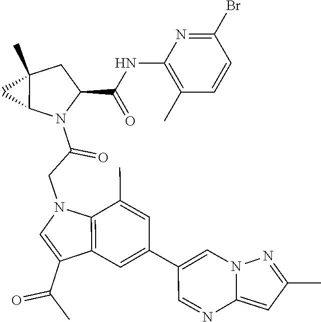 [SMILES: CC(=O)c1cn(CC(=O)N2[C@H](C(=O)Nc3nc(Br)ccc3C)C[C@@]3(C)C[C@@H]23)c2c(C)cc(-c3cnc4cc(C)nn4c3)cc12]

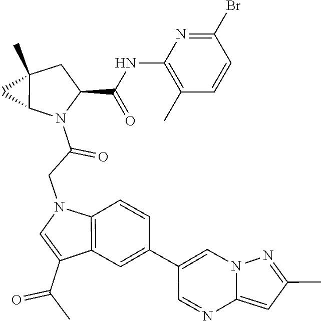 [SMILES: CC(=O)c1cn(CC(=O)N2[C@H](C(=O)Nc3nc(Br)ccc3C)C[C@@]3(C)C[C@@H]23)c2ccc(-c3cnc4cc(C)nn4c3)cc12]

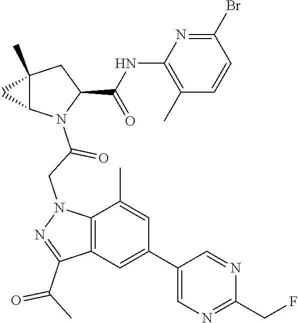 [SMILES: CC(=O)c1nn(CC(=O)N2[C@H](C(=O)Nc3nc(Br)ccc3C)C[C@@]3(C)C[C@@H]23)c2c(C)cc(-c3cnc(CF)nc3)cc12]

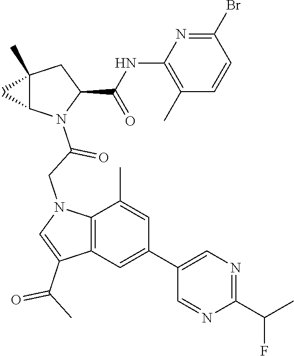 [SMILES: CC(=O)c1cn(CC(=O)N2[C@H](C(=O)Nc3nc(Br)ccc3C)C[C@@]3(C)C[C@@H]23)c2c(C)cc(-c3cnc(C(C)F)nc3)cc12]

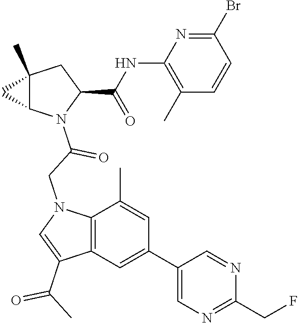 [SMILES: CC(=O)c1cn(CC(=O)N2[C@H](C(=O)Nc3nc(Br)ccc3C)C[C@@]3(C)C[C@@H]23)c2c(C)cc(-c3cnc(CF)nc3)cc12]

In some embodiments, the compound of formula (V) is a compound disclosed in WO2021/168320A1. For example, the compound of formula (V) is a compound of Table 4 or a pharmaceutically acceptable salt thereof.

TABLE 4

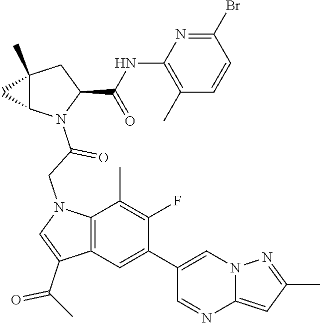 [SMILES: CC(=O)c1cn(CC(=O)N2[C@H](C(=O)Nc3nc(Br)ccc3C)C[C@@]3(C)C[C@@H]23)c2c(C)c(F)c(-c3cnc4cc(C)nn4c3)cc12]

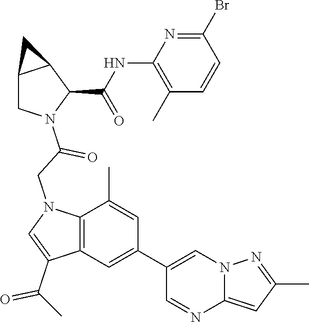 [SMILES: CC(=O)c1cn(CC(=O)N2C[C@H]3C[C@H]3[C@H]2C(=O)Nc2nc(Br)ccc2C)c2c(C)cc(-c3cnc4cc(C)nn4c3)cc12]

In some embodiments, the compound of formula (V) is a compound disclosed in WO2017/035409A1. For example, the compound of formula (V) is a compound of Table 5 or a pharmaceutically acceptable salt thereof.

TABLE 5

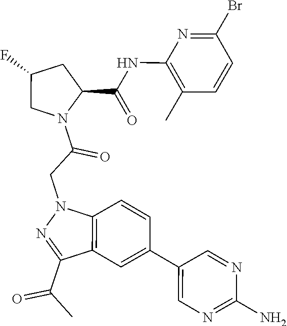 [SMILES: CC(=O)c1nn(CC(=O)N2C[C@H](F)C[C@H]2C(=O)Nc2nc(Br)ccc2C)c2ccc(-c3cnc(N)nc3)cc12]

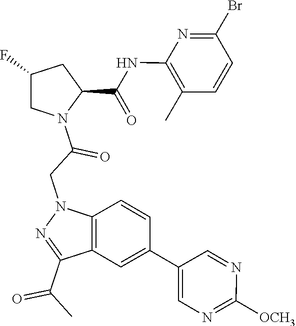 [SMILES: COc1ncc(-c2ccc3c(c2)c(C(C)=O)nn3CC(=O)N2C[C@H](F)C[C@H]2C(=O)Nc2nc(Br)ccc2C)cn1]

### EXAMPLES

The examples described herein serve to illustrate the present disclosure, and the disclosure is not limited to the examples given.

### Example 1: Synthesis of 6-Bromo-3-Methylpyridine-2-Amine

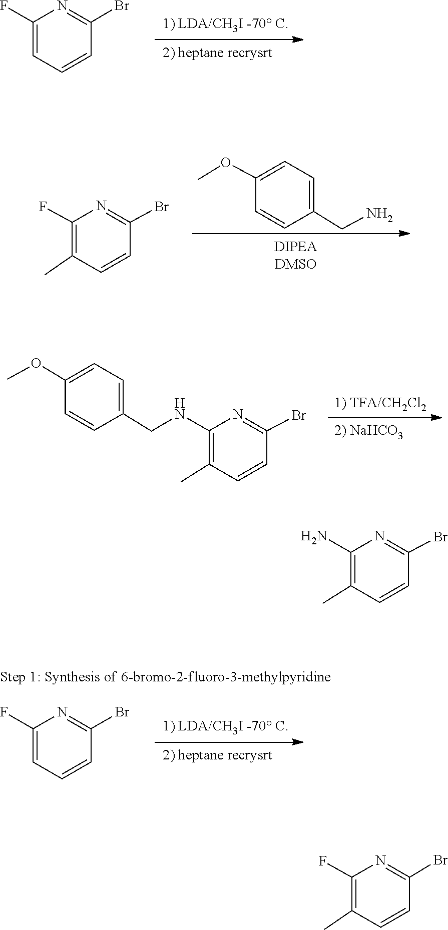

2-bromo-6-fluoropyridine (15 g, 85.233 mmol, 1 equiv.) was dissolved in tetrahydrofuran (225 mL, 15 vols.) and cooled to −70° C. Lithium diisopropylamide (55.402 mL, 110.803 mmol, 1.3 equiv. 2M solution) was slowly added to the solution, which was allowed to stir for 2.5 hr at −70° C. Iodomethane (18.147 g, 1.5 equiv.) was then slowly added, and the reaction mixture stirred overnight and allowed to warm to room temperature. Aqueous NH₄Cl was subsequently added, and the tetrahydrofuran was removed under reduced pressure to leave a residue. The residue thus obtained was extracted with CH₂Cl₂, after which the CH₂Cl₂ was removed. To the remaining residue was added 75 mL heptane, stirred, cooled to 5° C. and then filtered. The collected crystals were washed with cold heptane (30 mL) and dried to afford 6-bromo-2-fluoro-3-methylpyridine (10.06 g, 52.94 mmol, yield: 62.12%).

### Step 2: Synthesis of 6-bromo-N-(4-methoxybenzyl)-3-methylpyridin-2-amine

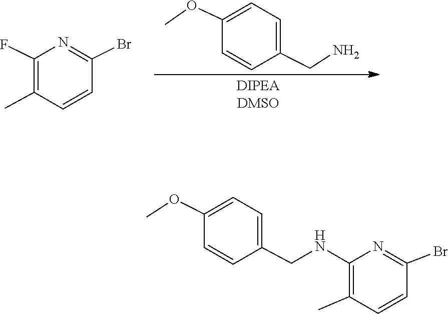

A solution of 6-bromo-2-fluoro-3-methylpyridine (10.06 g, 52.943 mmol, 1 equiv.), 4-methoxybenzylamine (14.525 g, 2 equiv.), Hunig's Base (20.528 g, 3 equiv.) in dimethyl sulfoxide (100 mL, 10 vols.) was bubbled with Ar for ~10 min then heated at 110° C. overnight. The reaction mixture was extracted with water and AcOEt. The water layer was again extracted with AcOEt and then combined AcOEt extract was washed with 1M citric acid aq followed by water wash. Then washed with sat. NaHCO₃aq and dried over Na₂SO₄ and removed solvent. The residue was used directly for the next reaction without purification.

### Step 3: Synthesis of 6-bromo-3-methylpyridin-2-amine

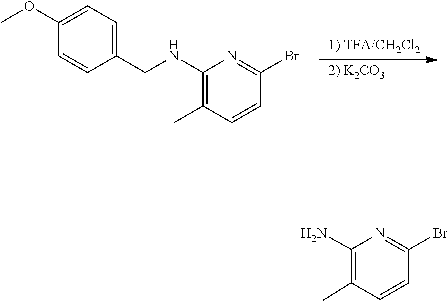

To the residue from step 2 was added trifluoroacetic acid (50 mL). The mixture was stirred for 4 hr at room temperature, then the trifluoroacetic acid was removed in vacuo. CH₂Cl₂ (100 mL) and water (~50 mL) were added to the reaction mixture, after which K₂CO₃ was slowly added to basify the mixture basic. The reaction mixture was then extracted with CH₂Cl₂, and the organic layer was dried over Na₂SO₄ then filtered, and the solvent was removed under reduced pressure. Heptane (20-30 mL) was then added to the residue, and the suspension was stirred for ~1 hr at room temperature then filtered. The filtered solid was washed with heptane twice to afford 6-bromo-3-methylpyridin-2-amine (6.98 g, yield 70.5%, 2 steps). LC/MS (ESI) (m/z): 187/189 (M+H)⁺.

### OTHER EMBODIMENTS

Various modifications and variations of the described compounds and methods of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosed methods that are obvious to those skilled in the art are intended to be within the scope of the disclosure.

Other embodiments are in the claims.

1. A method of preparing a compound of formula (A): 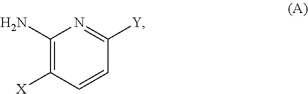
or a salt thereof, wherein
X is optionally substituted C₁-C₆ alkyl or optionally substituted C₃-C₈ cycloalkyl; and
Y is halo; cyano; OH; optionally substituted C₁-C₆ alkyl; optionally substituted C₁-C₆ alkoxyl; optionally substituted C₃-C₈ cycloalkyl; optionally substituted C₆-C₁₀ aryl; optionally substituted 4- to 10-membered saturated or unsaturated non-aromatic heterocyclyl containing 1-4 heteroatoms selected from N, O, and S; optionally substituted 5- to 10-membered heteroaryl containing 1, 2, or 3 heteroatoms selected from N, O, and S; optionally substituted C₁-C₆ thioalkyl; OR_{y1}, wherein R_{y1} is optionally substituted 5- to 10-membered heteroaryl containing 1, 2, or 3 heteroatoms selected from N, O, and S or optionally substituted 4- to 10-membered saturated or unsaturated non-aromatic heterocyclyl containing 1-4 heteroatoms selected from N, O, and S; or C(O)R_{y2}, wherein R_{y2} is H, optionally substituted C₁-C₆ alkyl, optionally substituted C₃-C₈ cycloalkyl, or optionally substituted C₆-C₁₀ aryl;
the method comprising:
(i) providing a compound of formula (B); 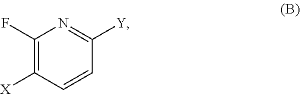
wherein X and Y are as defined for formula (A);
(ii) coupling the compound of formula (B) to a compound of formula (C):
P¹NH₂ (C),
wherein P¹ is H, optionally substituted C₁-C₆ alkyl, or a N-protecting group;
to form a compound of formula (D): 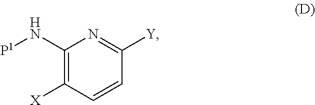
wherein all variables are as defined for formulas (B) and (C); and
(iii) (a) when P¹ is optionally substituted C₁-C₆ alkyl, reacting the compound of formula (D) or the salt thereof with a Brønsted acid to form the compound of formula (A) or the acid addition salt thereof; or (b) when P¹ is a N-protecting group, reacting the compound of formula (D) with a N-protecting-group-removing agent to form the compound of formula (A) or the acid addition salt thereof.

2. The method of claim 1, wherein P¹ is H.

3. The method of claim 1, wherein P¹ is optionally substituted C₁-C₆ alkyl.

4. The method of claim 3, wherein P¹ is tert-butyl.

5. The method of claim 1, wherein P¹ is a N-protecting group.

6. The method of claim 5, wherein P¹ is optionally substituted benzyl.

7. The method of claim 6, wherein P¹ is 4-methoxybenzyl.

8. The method of any one of claim 1 or 3-7, wherein the compound of formula (D) is reacted with a Brønsted acid to form an acid addition salt of the compound of formula (A).

9. The method of claim 8, wherein the Brønsted acid is trifluoroacetic acid.

10. The method of any one of claims 1-9, wherein the amine is N,N-diisopropylethylamine.

11. The method of any one of claims 8-10, further comprising reacting the acid addition salt of the compound of formula (A) with a base to obtain the compound of formula (A) as a free base.

12. The method of claim 11, wherein the base is NaHCO₃.

13. The method of any one of claims 1-12, wherein said coupling the compound of formula (B) or the salt thereof to the compound of formula (D) or the salt thereof is performed with the compound of formula (B) or the salt thereof and the compound of formula (D) or the salt thereof at a ratio of about 1:2.

14. The method of any one of claims 1-13, wherein said providing the compound of formula (B) comprises:
(i) providing a compound of formula (E): 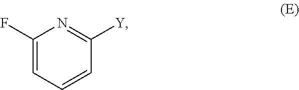
wherein Y is as defined for formula (A); and
(ii) reacting the compound of formula (E) or the salt thereof with a non-nucleophilic strong base, followed by an alkylation reagent comprising X, to obtain the compound of formula (B).

15. The method of claim 14, wherein the non-nucleophilic strong base is a lithium amide.

16. The method of claim 15, wherein the non-nucleophilic strong base is lithium diisopropylamide.

17. The method of claim 14, wherein the strong base is a potassium amide.

18. The method of claim 17, wherein the strong base is potassium bis(trimethylsilyl)amide.

19. The method of any one of claims 14-18, wherein the alkylation reagent comprising X is an alkyl halide.

20. The method of claim 19, wherein the alkylation reagent is methyl iodide.

21. The method of any one of claims 1-20, further comprising the compound of formula (A) or the salt thereof to a compound of formula (I): 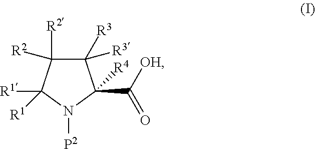
or a salt thereof, wherein
each of R¹, R^{1′}, R², R^{2′}, R³, R^{3′}, and R⁴ is, independently, H; cyano; halo; OH; nitro; optionally substituted C₁-C₆ alkyl; optionally substituted C₂-C₆ alkenyl; C₁-C₆ alkoxy; C₁-C₆ thioalkyl; optionally substituted C₃-C₈ carbocyclyl; optionally substituted C₃-C₈ carbocyclyloxy;
—NRₐRₐ′; —C(O)NRₐRₐ′; —OC(O)NRₐRₐ′; —NRₐC(O)R_{b}; —NRₐC(O)OR_{b}; —C(O)R_{b}; —C(O)OR_{b}; —C═NR_{b}; or C═NOH, wherein each of Rₐ, Rₐ′, and R_{b} is independently selected from H, optionally substituted C₁-C₆ alkyl, optionally substituted C₂-C₆ alkenyl, optionally substituted C₂-C₆ alkynyl, and optionally substituted C₃-C₈ carbocyclyl; or
R¹ and R₂, together with the atoms to which each is attached, form an optionally substituted C₃-C₆ cycloalkyl; or
R² and R³, together with the atoms to which each is attached, form an optionally substituted C₃-C₆ cycloalkyl; or
R¹ and R₃ or R² and R⁴ combine to form an optionally substituted C₁-C₂ alkylene; or
R¹ and R_{1′}, R² and R^{2′}, or R₃ and R_{3′} combine to form oxo; and
P² is a N-protecting group,
to form a compound of formula (II): 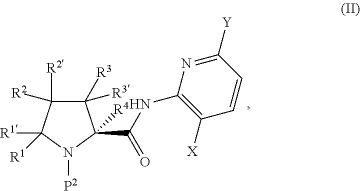
or a salt thereof, wherein all variables are as defined for X and Y are as defined for formula (A); and
R¹, R^{1′}, R², R^{2′}, R³, R^{3′}, R⁴, and P² are as defined for formula (I).

22. The method of claim 21, further comprising reacting the compound of formula (II) or the salt thereof with a N-protecting-group-removing agent to form a compound of formula (III): 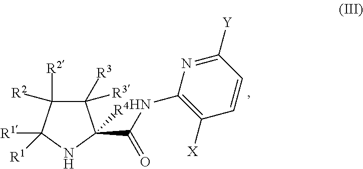
or a salt thereof, wherein X and Y are as defined for formula (A); and R¹, R^{1′}, R², R^{2′}, R³, R^{3′}, and R₄, are as defined for formula (I).

23. The method of claim 22, further comprising coupling the compound of formula (III) or the salt thereof to a compound of formula (IV): 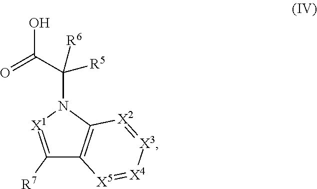
or a salt thereof, wherein
each of R₅ and R₆ is independently H or optionally substituted C₁-C₆ alkyl;
R⁷ is H; halo; OH; NH₂; cyano; optionally substituted C₁-C₆ alkyl; optionally substituted C₂-C₆ alkenyl; optionally substituted 3- to 8-membered heterocyclyl; —C(O)NR_{c}R_{c}′, wherein each of R_{c} and R_{c}′ is independently H, optionally substituted C₁-C₆ alkyl, optionally substituted C₂-C₆ alkenyl, optionally substituted C₂-C₆ alkynyl, optionally substituted C₁-C₆ alkoxyl, or optionally substituted C₃-C₆ cycloalkyl;
—C(O)R_{d}; —OC(O)R_{d}; or —C(O)OR_{d}; wherein R_{d}, in each instance, is selected from H, optionally substituted C₁-C₆ alkyl, optionally substituted C₁-C₆ alkoxy, and optionally substituted C₃-C₈ carbocyclyl;
X¹ is N or CRₑ, wherein Rₑ is H, halo, optionally substituted C₁-C₆ alkyl, or optionally substituted C₁-C₆ alkoxy;
each of X² and X⁵ is independently N or CR_{f}, wherein each R_{f} is independently selected from H, halo, cyano, optionally substituted C₁-C₆ alkyl, optionally substituted C₁-C₆ alkoxy, optionally substituted C₃-C₈ carbocyclyl, and optionally substituted 5- to 8-membered heteroaryl; and
each of X³ and X⁴ is independently selected from N, CR_{g}, and CRₕ, wherein R_{g} is selected from H, halo, cyano, optionally substituted C₁-C₆ alkyl, optionally substituted C₁-C₆ alkoxy, and —C(O)ORᵢ, wherein Rᵢ is H or optionally substituted C₁-C₆ alkyl; and Rₕ is selected from optionally substituted C₄-C₁₀ aryl, optionally substituted 5- to 10-membered heteroaryl containing 1, 2, or 3 heteroatoms selected from N, O, and S, and optionally substituted 4- to 10-membered saturated or unsaturated non-aromatic heterocyclyl containing 1-4 heteroatoms selected from N, O, and S;
wherein at least one of X³ and X⁴ is CRₕ,
to form a compound of formula (V): 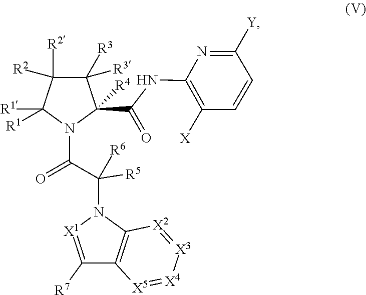
or a pharmaceutically acceptable salt thereof, wherein X and Y are as defined for formula (A); R¹, R^{1′}, R², R^{2′}, R³, R^{3′}, and R⁴ are as defined for formula (II); and all other variables are as defined for formula (IV).

24. The method of any one of claims 21-23, wherein P² is tert-butoxycarbonyl.

25. The method of any one of claims 22-24, wherein the N-protecting-group-removing agent is hydrogen chloride, and said reacting the compound of formula (II) with the N-protecting-group-removing agent forms a hydrochloride salt of the compound of formula (III).

26. The method of claim 25, wherein the hydrochloride salt of the compound of formula (III) is coupled to the compound of formula (IV) in dimethylformamide in the presence of 1-[bis(dimethylamino)methylene]-1H-1,2,3-triazolo[4,5-b]pyridinium 3-oxide hexafluorophosphate and N, N-diisopropylethylamine.

27. The method of any one of claims 22-24, wherein the N-protecting-group-removing agent is hydrogen bromide, and said reacting the compound of formula (II) with the N-protecting-group-removing agent forms a hydrobromide salt of the compound of formula (III).

28. The method of claim 27, wherein the hydrobromide salt of the compound of formula (III) is coupled to the compound of formula (IV) in acetonitrile in the presence of propanephosphonic acid anhydride and N, N-diisopropylethylamine.

29. The method of any one of claims 22-24, wherein the N-protecting-group-removing agent is trifluoroacetic acid, and said reacting the compound of formula (II) with the N-protecting-group-removing agent forms a trifluoroacetic acid salt of the compound of formula (III).

30. The method of claim 29, wherein the trifluoroacetic acid salt of the compound of formula (III) is coupled to the compound of formula (IV) in dimethylformamide in the presence of N, N-diisopropylethylamine and 1-[bis(dimethylamino)methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate.

31. The method of any one of claims 21-30, wherein R₂ is fluoro.

32. The method of claim 31, wherein each of R¹, R^{1′}, R³, R^{3′}, and R⁴ is hydrogen.

33. The method of claim 32, wherein

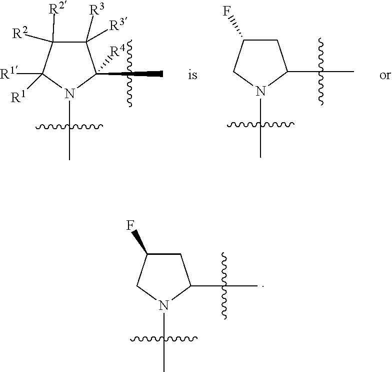

34. The method of claim 33, wherein 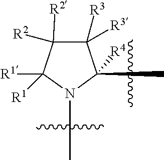
is

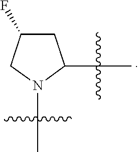 [SMILES: *C1C[C@@H](F)CN1*]

35. The method of claim 31, wherein R^{2′} is optionally substituted C₁-C₆ alkyl.

36. The method of claim 35, wherein 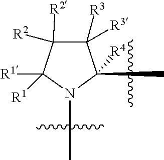
is:

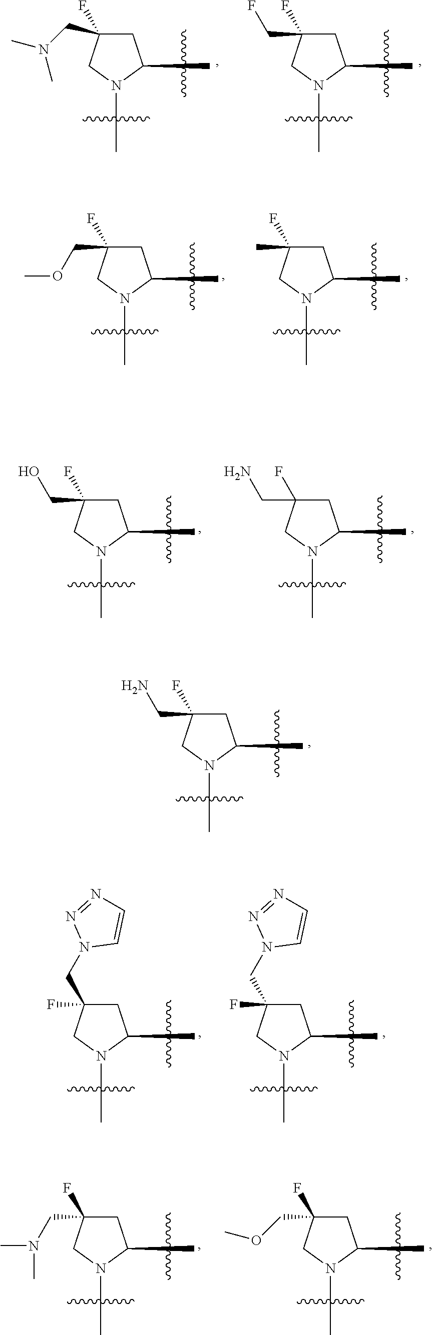

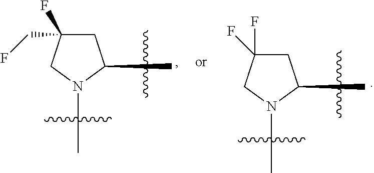

37. The method of claim 31, wherein 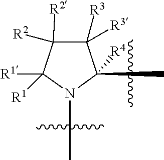
is:

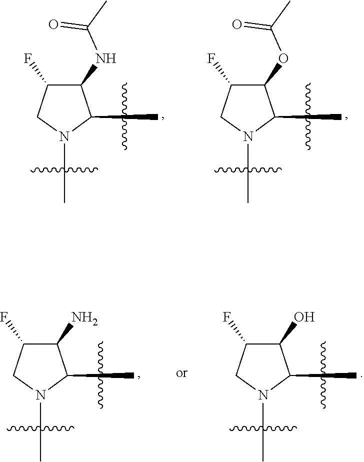

38. The method of any one of claims 21-30, wherein R¹ and R₂, together with the atoms to which each is attached, form an optionally substituted C₃-C₆ cycloalkyl; and each of R^{1′}, R³, R^{3′}, and R⁴ is H.

39. The method of claim 38, wherein R¹ and R₂, together with the atoms to which each is attached, form an optionally substituted cyclopropyl.

40. The method of claim 38 or 39, wherein R^{2′} is H.

41. The method of claim 40, wherein 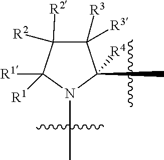
is:

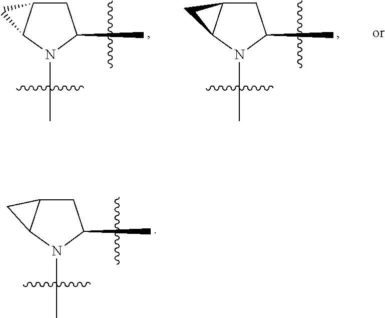

42. The method of claim 38 or 39, wherein R₂ is optionally substituted C₁-C₆ alkyl.

43. The method of claim 32, wherein 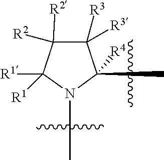
is:

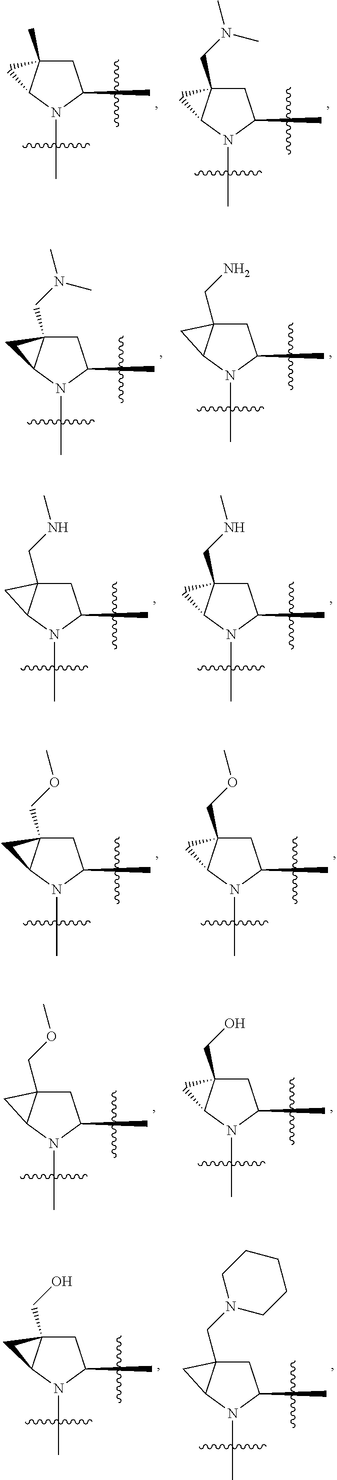

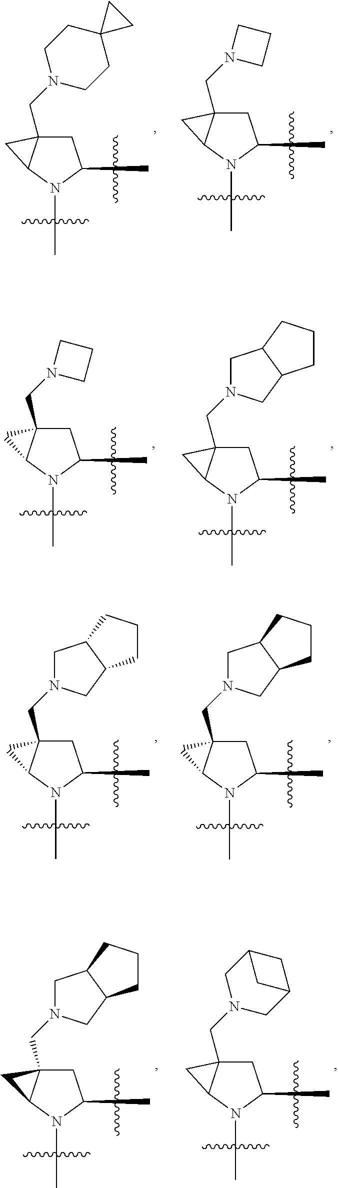

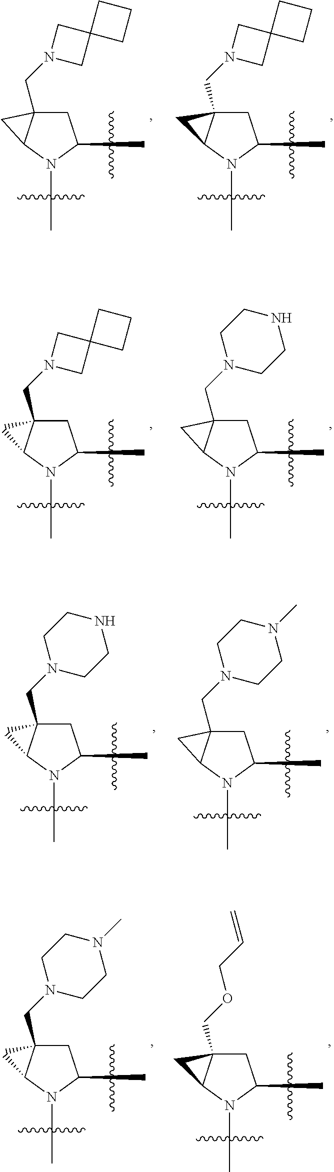

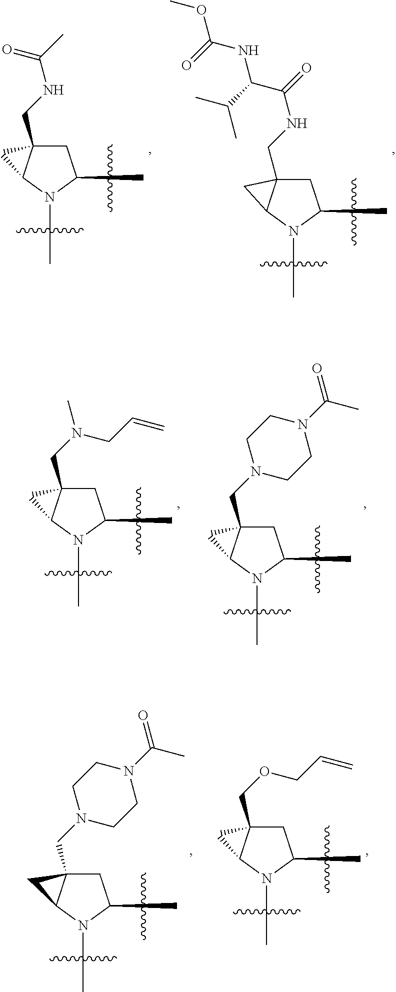

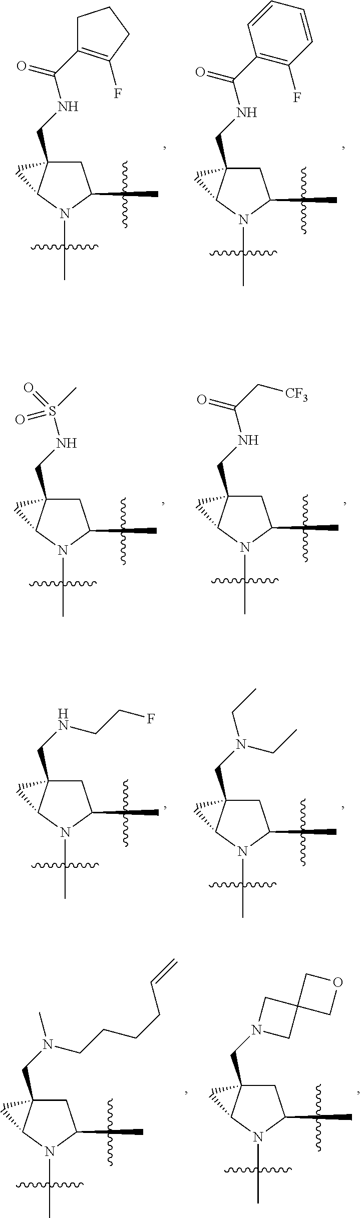

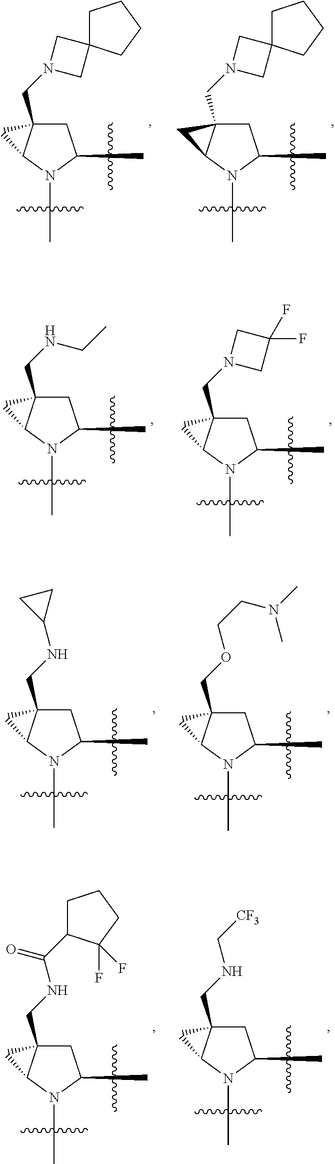

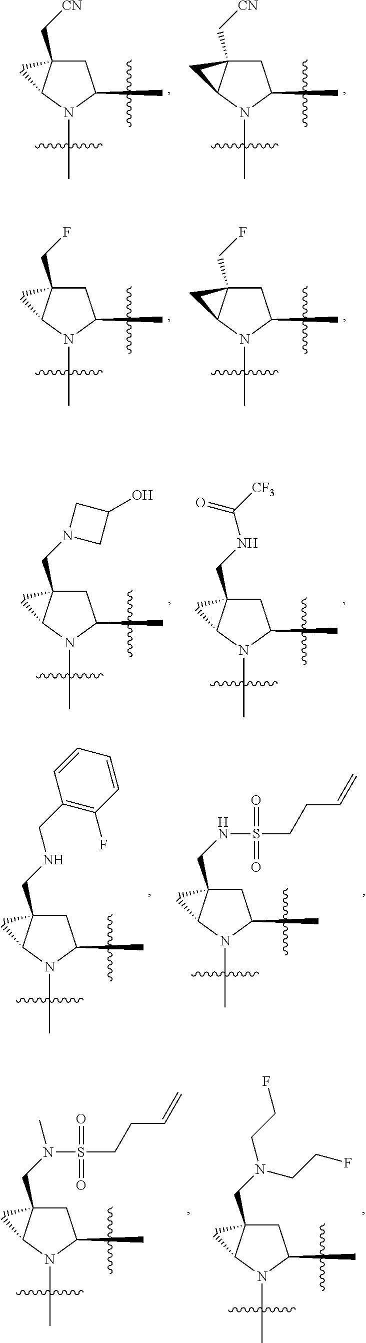

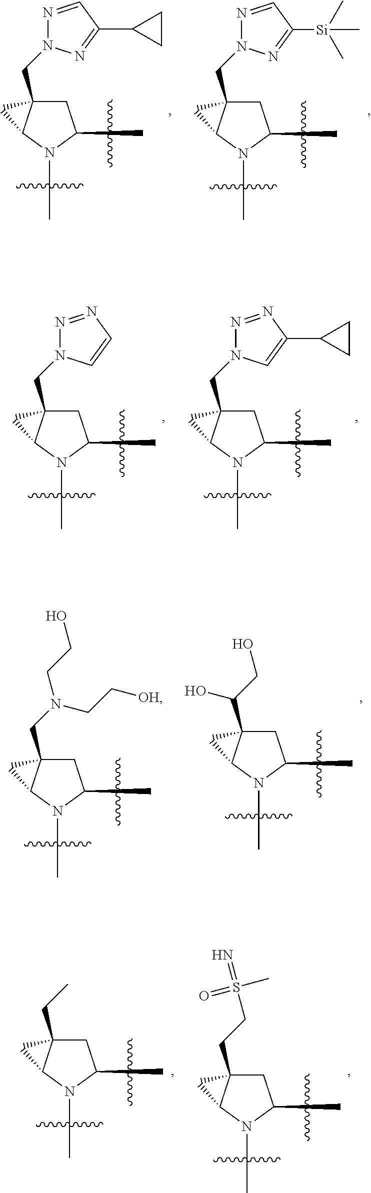

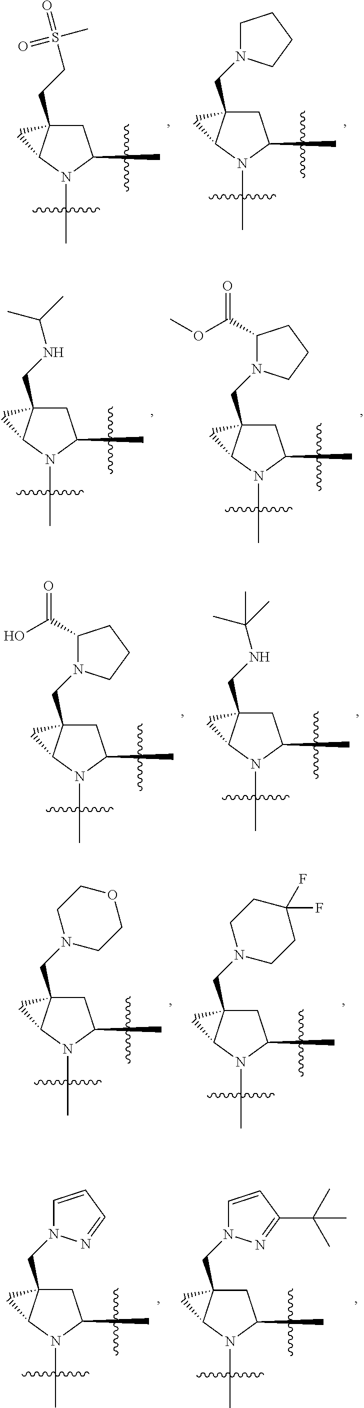

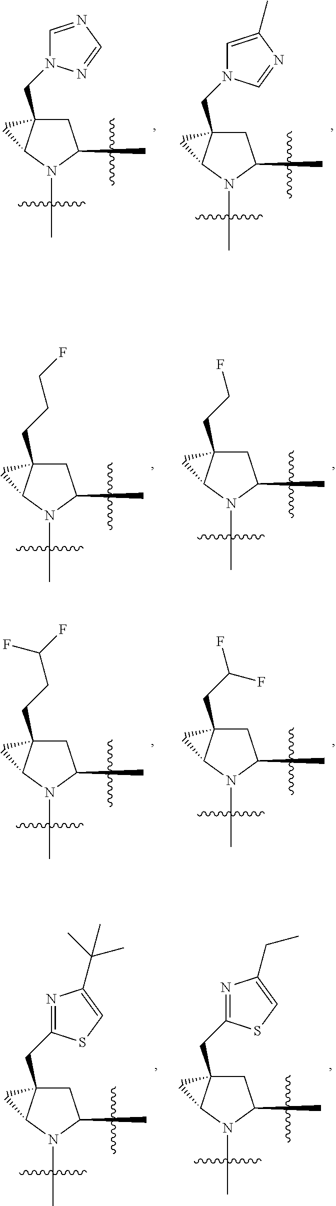

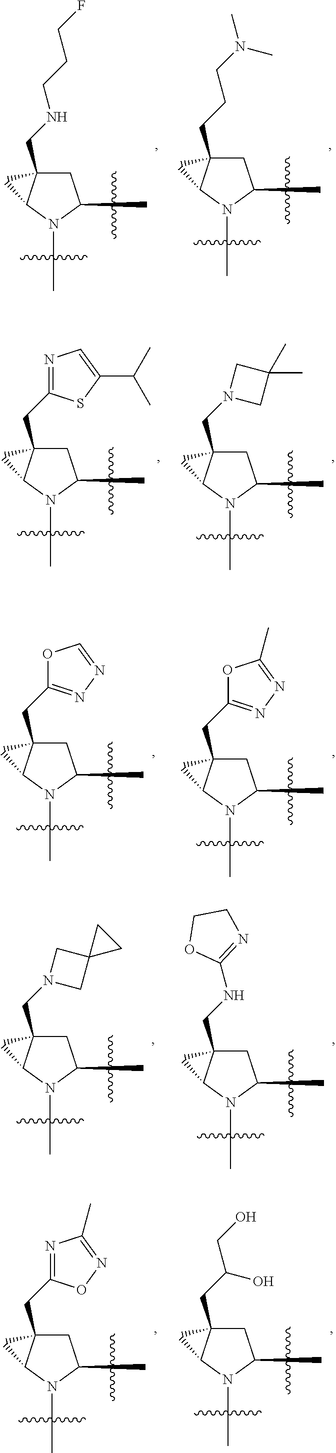

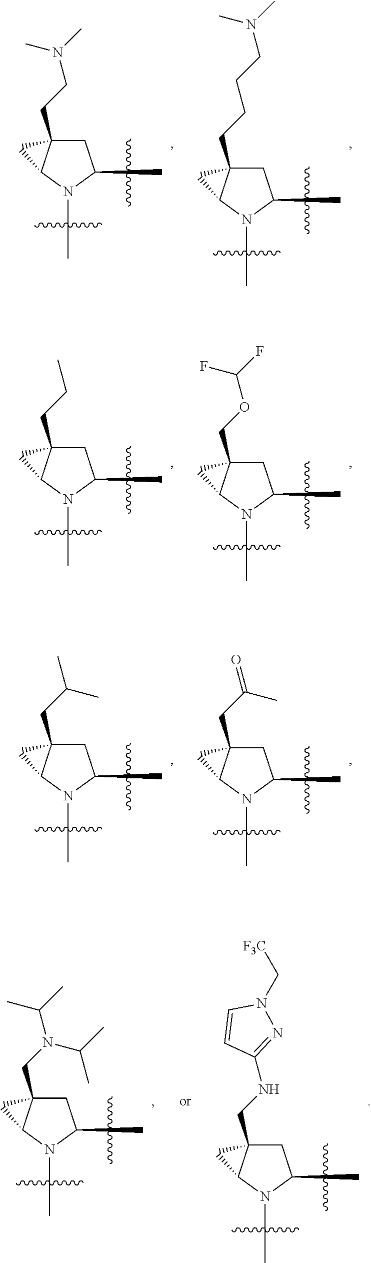

44. The method of claim 42 or 43, wherein 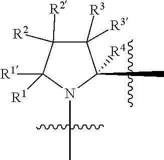
is

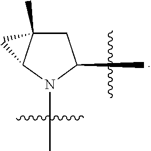 [SMILES: *N1[C@H](C)C[C@@]2(C)C[C@@H]12]

45. The method of claim 38 or 39, wherein 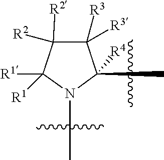
is:

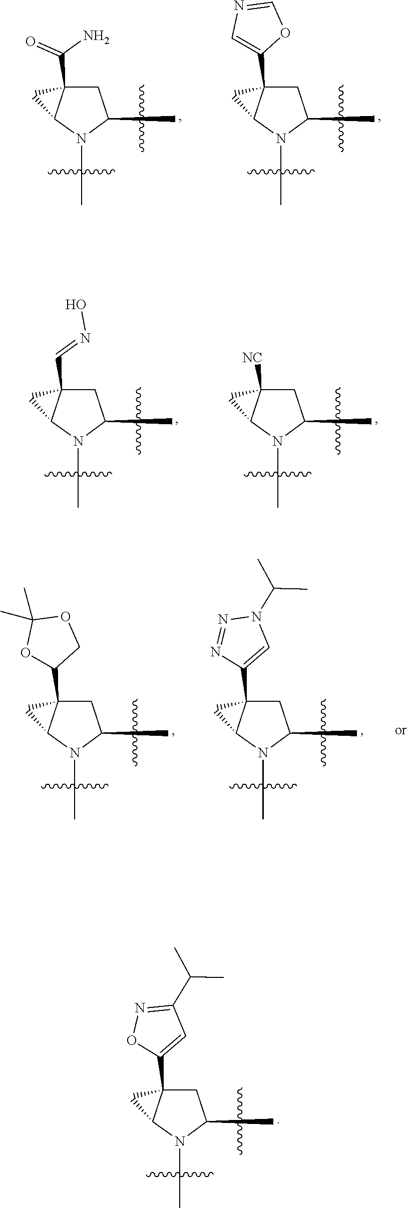

46. The method of any one of claims 21-30, wherein R² and R³, together with the atoms to which each is attached, form an optionally substituted C₃-C₆ cycloalkyl, and each of R¹, R^{1′}, R^{2′}, R^{3′}, and R⁴ is H.

47. The method of claim 46, wherein R² and R³, together with the atoms to which each is attached, form an optionally substituted cyclopropyl.

48. The method of claim 47, wherein 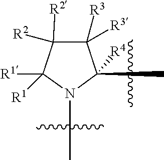
is

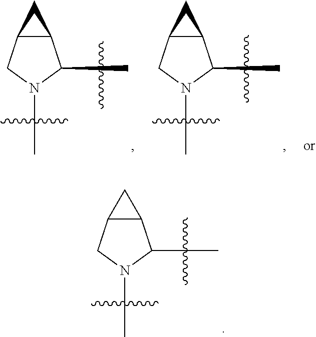

49. The method of any one of claims 21-30, wherein R² and R⁴ combine to form optionally substituted C₁-C₂ alkylene.

50. The method of claim 49, wherein 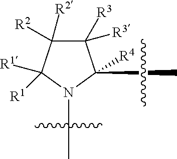
is

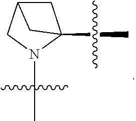

51. The method of any one of claims 21-30, wherein 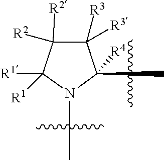
is:

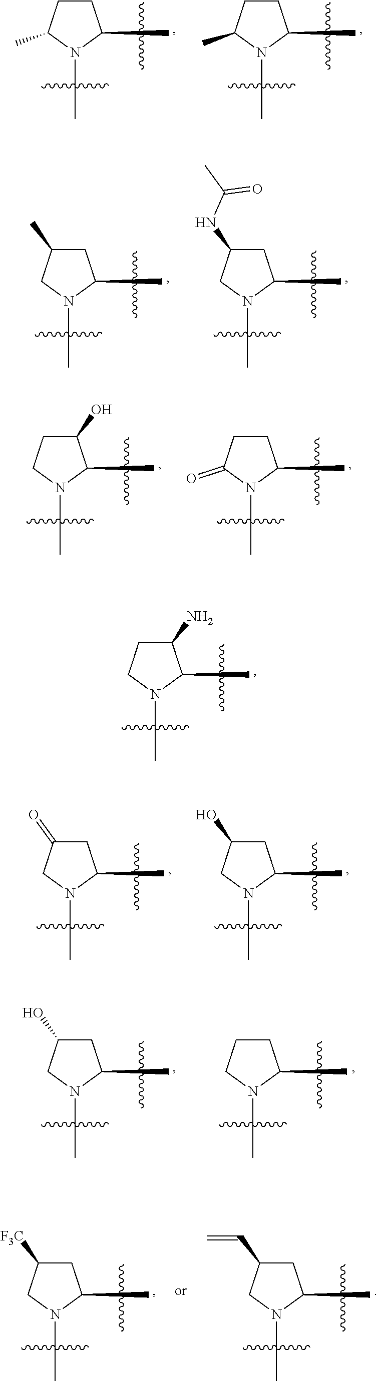

52. The method of any one of claims 23-51, wherein X¹ is N.

53. The method of any one of claims 23-51, wherein X¹ is CRₑ.

54. The method of claim 53, wherein X¹ is C(CH₃).

55. The method of claim 53, wherein X¹ is CH.

56. The method of any one of claims 23-55, wherein X² is CR_{f}.

57. The method of claim 56, wherein R_{f} is H or optionally substituted C₁-C₆ alkyl.

58. The method of claim 56 or 57, wherein X² is CH.

59. The method of claim 56 or 57, wherein X² is C(CH₃).

60. The method of any one of claims 23-59, wherein X⁵ is CR_{f}.

61. The method of claim 60, wherein X⁵ is CH.

62. The method of any one of claims 23-61, wherein X³ is CRₕ.

63. The method of claim 62, wherein X⁴ is N.

64. The method of claim 62, wherein X⁴ is CH.

65. The method of any one of claims 23-61, wherein X⁴ is CRₕ.

66. The method of claim 65, wherein X³ is N.

67. The method of claim 65, wherein X³ is CH.

68. The method of any one of claims 23-67, wherein Rₕ is optionally substituted 5- to 10-membered heteroaryl containing 1, 2, or 3 heteroatoms selected from N, O, and S.

69. The method of claim 68, wherein Rₕ is 6-membered heteroaryl containing 1, 2, or 3 heteroatoms selected from N, O, and S.

70. The method of claim 69, wherein Rₕ is optionally substituted pyrimidinyl.

71. The method of claim 70, wherein Rₕ is:

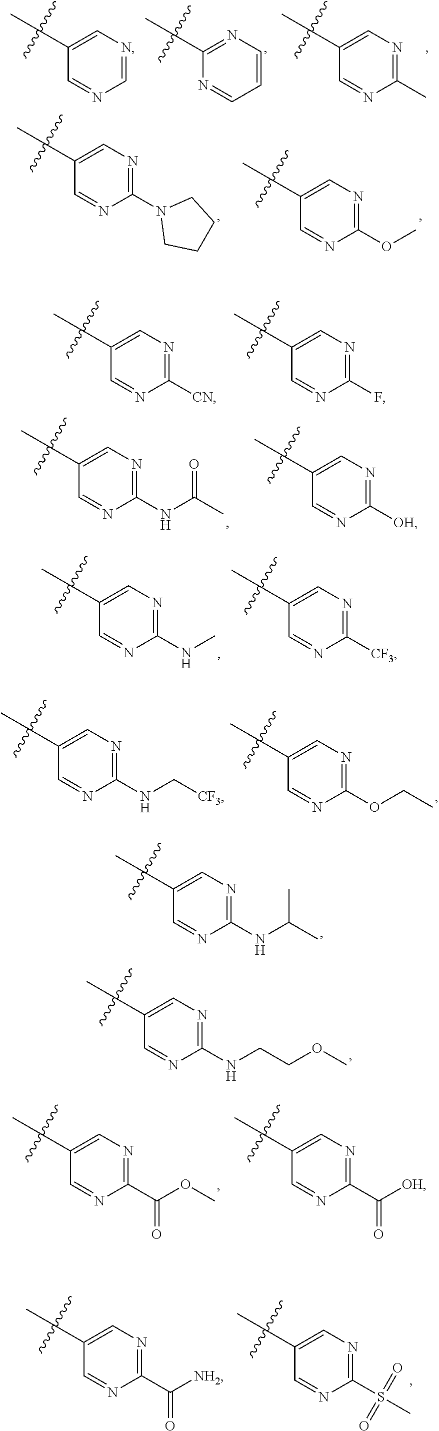

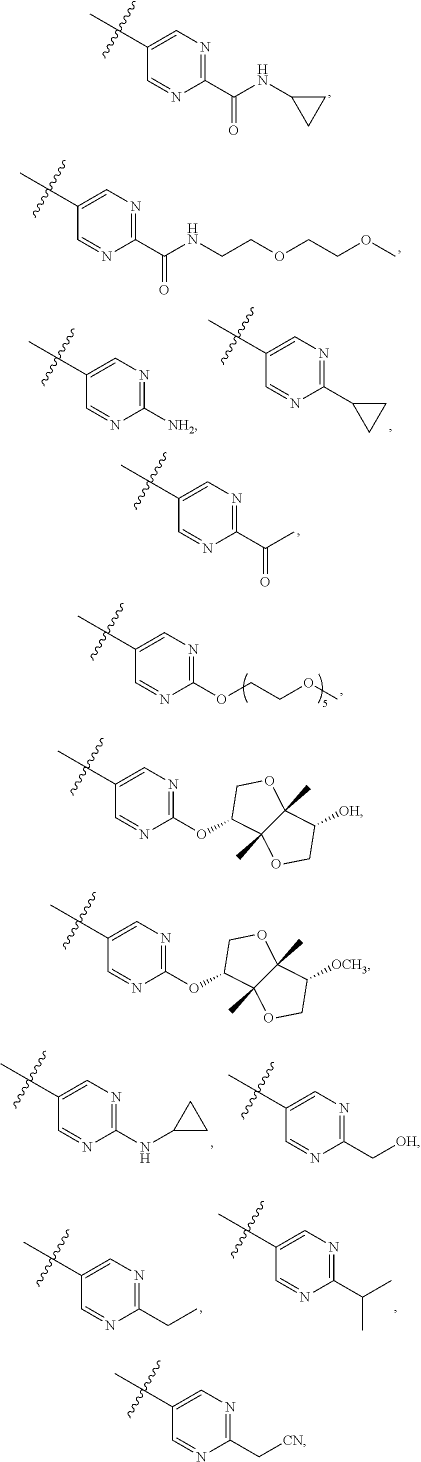

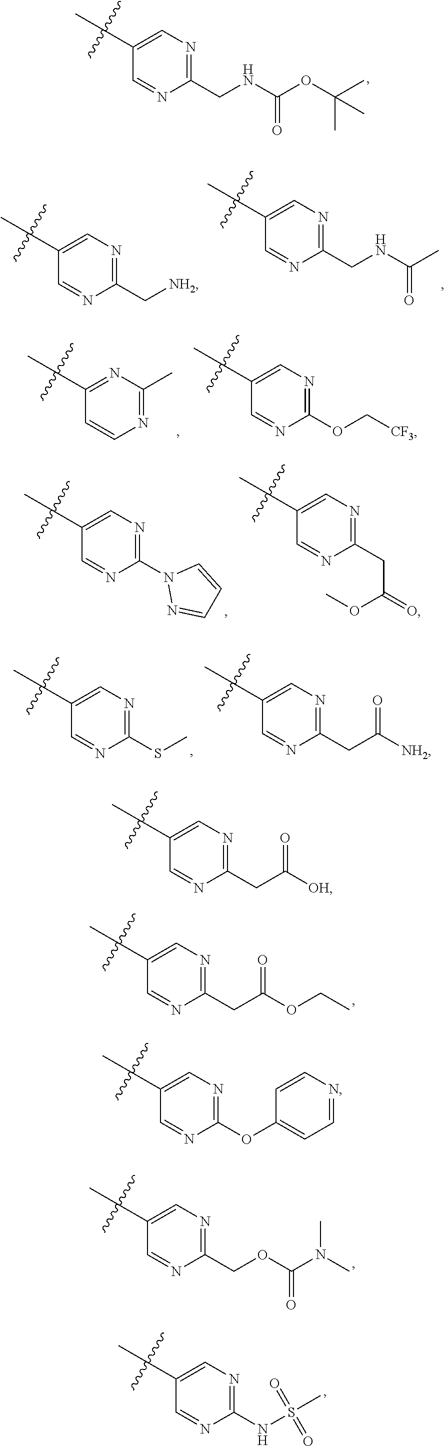

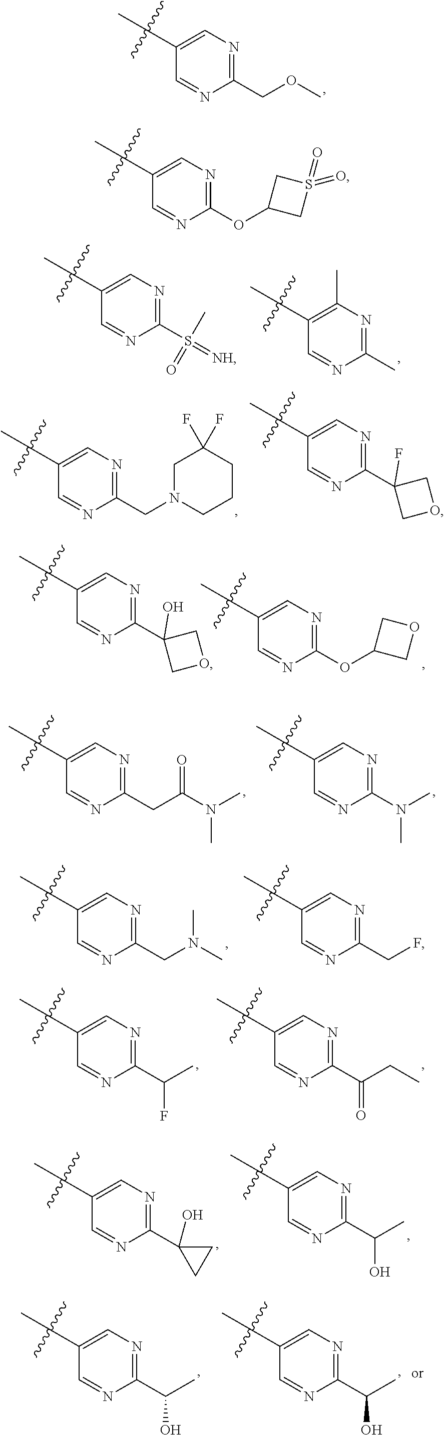

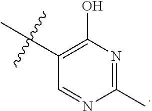 [SMILES: *c1cnc(C)nc1O]

72. The method of claim 71, wherein Rₕ is

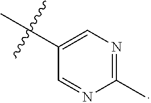 [SMILES: *c1cnc(C)nc1]

73. The method of claim 69, wherein Rₕ is:

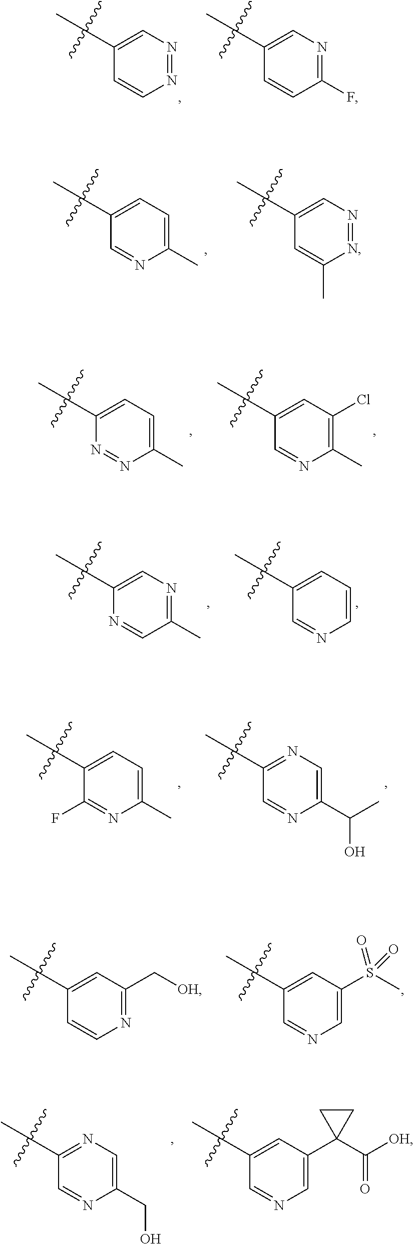

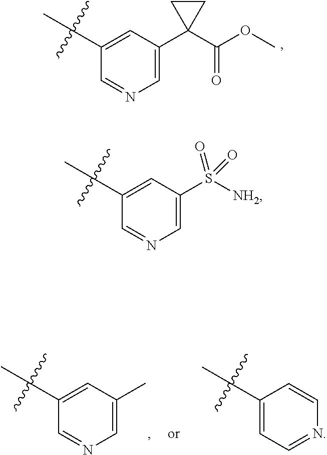

74. The method of claim 68, wherein Rₕ is optionally substituted 8- to 10-membered bicyclic heteroaryl containing 1, 2, or 3 heteroatoms selected from N, O, and S.

75. The method of claim 74, wherein Rₕ is optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl.

76. The method of claim 75, wherein Rₕ is:

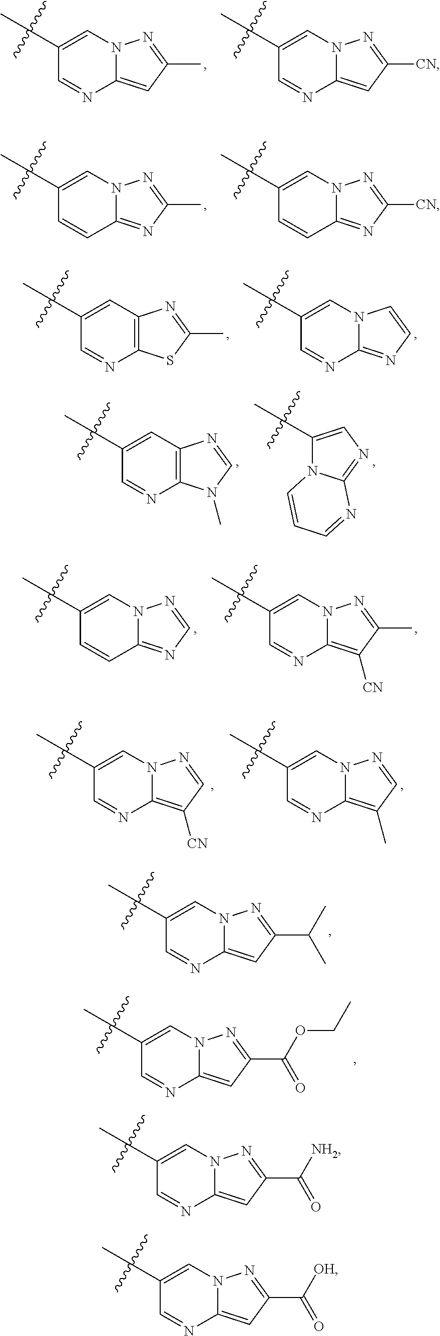

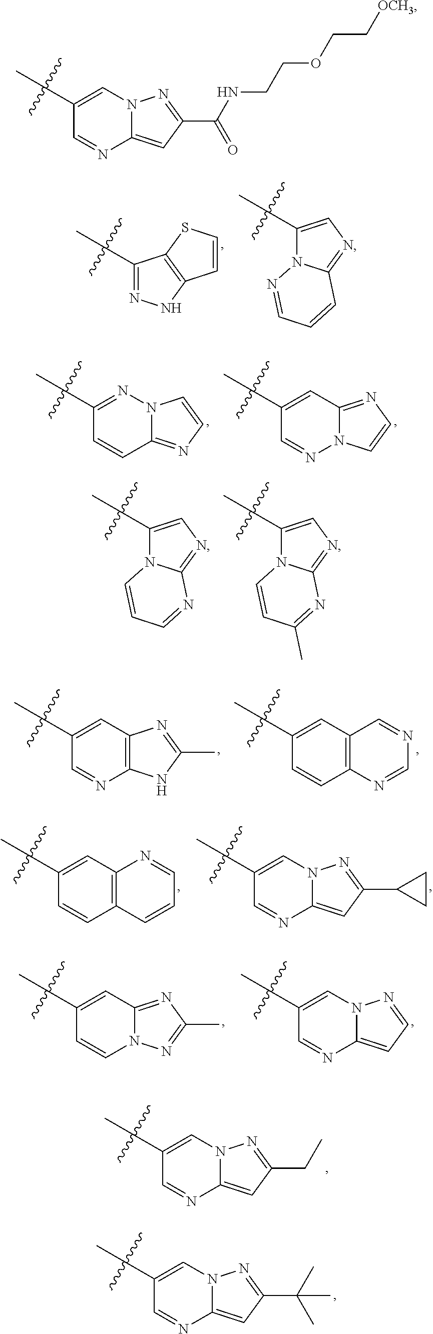

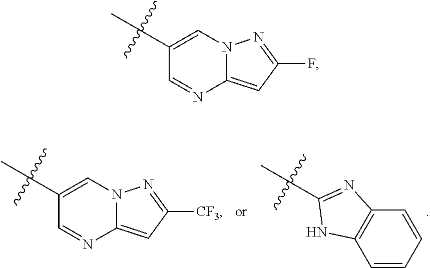 [SMILES: *c1cnc2cc(F)nn2c1 ; *c1cnc2cc(C(F)(F)F)nn2c1 ; *c1nc2ccccc2[nH]1]

77. The method of claim 76, wherein Rₕ is

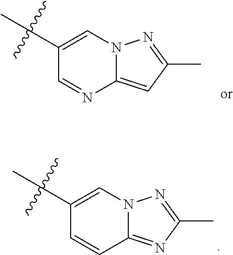 [SMILES: *c1cnc2cc(C)nn2c1 ; *c1ccc2nc(C)nn2c1]

78. The method of any one of claims 23-67, wherein Rₙ is optionally substituted C₆-C₁₄ aryl.

79. The method of claim 78, wherein Rₕ is optionally substituted phenyl.

80. The method of claim 79, wherein Rₕ is:

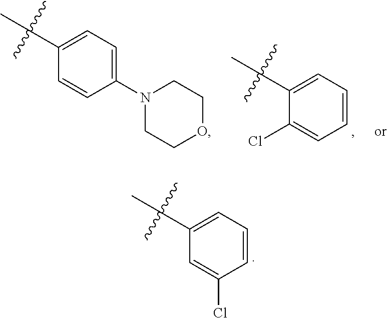 [SMILES: *c1ccc(N2CCOCC2)cc1 ; *c1ccccc1Cl ; *c1cccc(Cl)c1]

81. The method of any one of claims 23-67 wherein Rₕ is optionally substituted 6- to 9-membered unsaturated heterocyclyl containing 1-4 heteroatoms selected from N, O, or S.

82. The method of claim 81, wherein the Rₕ is bonded to the carbon atom to which it is attached through a carbon ring atom contained therein.

83. The method of claim 82, wherein Rₕ is:

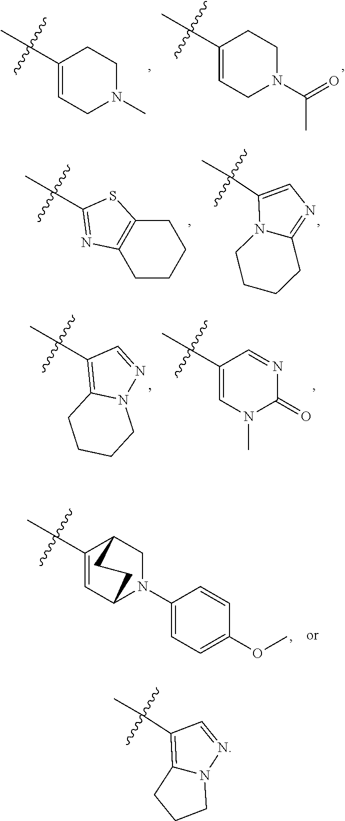

84. The method of claim 81, wherein Rₕ is:

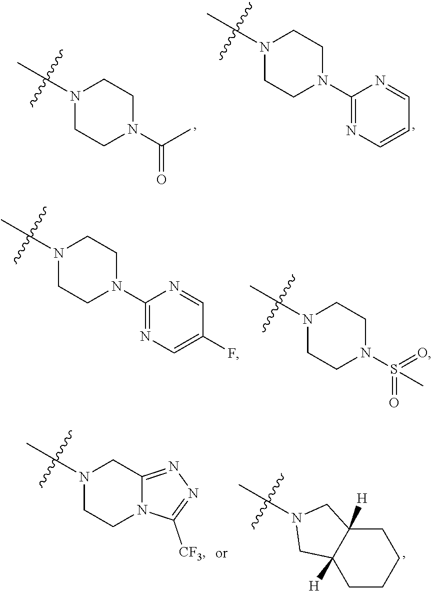

85. The method of claim 68, wherein Rₕ is optionally substituted 5-membered heteroaryl containing 1, 2, or 3 heteroatoms selected from N, O, and S.

86. The method of claim 85, wherein Rₕ is:

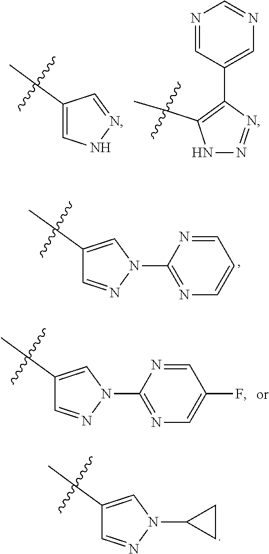 [SMILES: *c1cn[nH]c1 ; *c1[nH]nnc1-c1cncnc1 ; *c1cnn(-c2ncccn2)c1 ; *c1cnn(-c2ncc(F)cn2)c1 ; *c1cnn(C2CC2)c1]

87. The method of any one of claims 23-86, wherein R⁷ is —C(O)R_{d}.

88. The method of claim 87, wherein R⁷ is

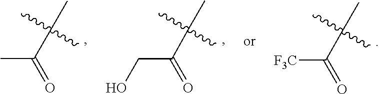

89. The method of claim 88, wherein R⁷ is

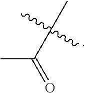 [SMILES: *C(C)=O]

90. The method of any one of claims 23-86, wherein R⁷ is —C(O)NR_{c}R_{c}′.

91. The method of claim 90, wherein R⁷ is:

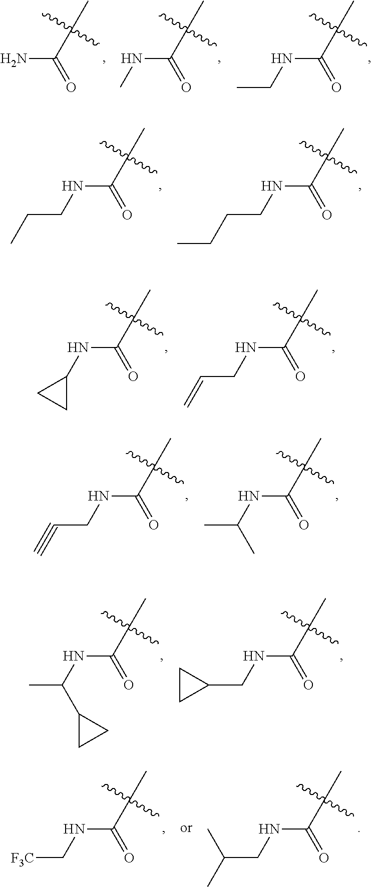

92. The method of claim 91, wherein R⁷ is

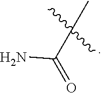 [SMILES: *C(N)=O]

93. The method of any one of claims 23-86, wherein R⁷ is —C(O)OR_{d}.

94. The method of claim 93, wherein R⁷ is —C(O)OCH₃ or —C(O)OH.

95. The method of any one of claims 23-86, wherein R⁷ is optionally substituted C₁-C₆ alkyl.

96. The method of claim 95, wherein R⁷ is

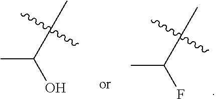

97. The method of any one of claims 23-86, wherein R⁷ is

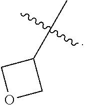 [SMILES: *C1COC1]

98. The method of any one of claims 23-86, wherein R⁷ is cyano.

99. The method of any one of claims 23-86, wherein R⁷ is halo.

100. The method of any one of claims 23-99, wherein R⁵ is H.

101. The method of any one of claims 23-100, wherein R⁶ is H.

102. The method of any one of claims 1-101, wherein Y is halo.

103. The method of claim 102, wherein Y is Br.

104. The method of any one of claims 1-103, wherein X is optionally substituted C₁-C₆ alkyl.

105. The method of claim 104, wherein X is methyl.

106. The method of any one of claims 23-30, wherein the compound of formula (V) is a compound of Table 1 or a pharmaceutically acceptable salt thereof.

107. The method of any one of claims 23-30, wherein the compound of formula (V) is a compound of Table 2 or a pharmaceutically acceptable salt thereof.

108. The method of any one of claims 23-30, wherein the compound of formula (V) is a compound of Table 3 or a pharmaceutically acceptable salt thereof.

109. The method of any one of claims 23-30, wherein the compound of formula (V) is a compound of Table 4 or a pharmaceutically acceptable salt thereof.
